(19) United States
(12) Patent Application Publication
(10) Pub. No.: US 2017/0267679 A1
(43) Pub. Date: Sep. 21, 2017

(54) TRICYCLIC FUSED PYRIMIDINE COMPOUNDS AS INHIBITORS OF p97 COMPLEX

(71) Applicant: Cleave Biosciences, Inc., Burlingame, CA (US)

(72) Inventors: Han-Jie Zhou, Foster City, CA (US); David Wustrow, Los Gatos, CA (US)

(21) Appl. No.: 15/458,955

(22) Filed: Mar. 14, 2017

Related U.S. Application Data
(60) Provisional application No. 62/308,533, filed on Mar. 15, 2016.

(51) Int. Cl. C07D 471/14

(52) U.S. Cl. CPC C07D 471/14

(57) ABSTRACT

Tricyclic fused pyrimidine compounds having an arylalkyl amine substituent at the P4 position and a substituted 1H-indol-1-yl, 1H-indol-3-yl, indanyl, indazol-1-yl, indazol-3-yl, benzotriazol-1-yl or 1H-benz[d]imidazol-1-yl group at the P2 position well as optional aliphatic, functional and/or aromatic components substituted at other positions of the tricyclic compounds of the invention. These compounds are inhibitors of the AAA proteasome complex containing p97 and are effective medicinal agents for treatment of diseases associated with p97 bioactivity such as cancer.

### CLAIM OF PRIORITY

This application claims the benefit of priority under 35 U.S.C. §119(e) to U.S. Provisional Patent Application Ser. No. 62/308,533, filed on Mar. 15, 2016, which is incorporated by reference herein in its entirety.

### BACKGROUND OF THE INVENTION

The AAA (ATPase Associated with a variety of Activities) ATPase p97 having the descriptive name, Valosin containing protein, is conserved across all eukaryotes and is essential for life in budding yeast (Giaever, G., et. al. Nature (2002) 418, 387-391) and mice (Muller, J. M. et al. Biochem. Biophys. Res. Commun. (2007) 354, 459-465). Humans bearing reduction-of-function alleles of p97 are afflicted with a syndrome that includes inclusion body myopathy and frontotemporal lobar degeneration (Weihl, C. et al. Hum. Mol. Genet. (2006) 15, 189-199). Loss-of-function studies in model organisms indicate that p97 plays a critical role in a broad array of cellular processes including Golgi membrane reassembly (Rabouille, C. et al. Cell (1995) 82, 905-914), membrane transport (Ye, Y. et al Nature (2001) 414, 652-656; Ye, Y. et al. Nature (2004) 429, 841-847) degradation of misfolded membrane and secretory proteins by the ubiquitin-proteasome system (UPS) (Golbik, R. et al. Biol. Chem. (1999) 380, 1049-1062; Richly, H. et al. Cell (2005) 120, 73-84), regulation of myofibril assembly (Janiesch, P. C. et al. Nat. Cell Biol. (2007) 9, 379-390), and cell division (Cao, K. et al. Cell (2003) 115, 355-367). The broad range of cellular functions for this protein is thought to derive from its ability to unfold proteins or disassemble protein complexes. The mechanochemical activity of p97 is linked to substrate proteins by an array of at least 14 UBX domain adapters that bind p97, as well as the non-UBX domain adaptors Ufdl and Np14 (Meyer, H. H. et al. EMBO J. (2000) 19, 2181-2192).

The sequence of p97 reveals three domains (N-domain, D1 ATPase domain, and D2 ATPase domain) joined by linker regions. X-ray crystallography of p97 revealed that it forms a homohexamer of 97 kilodalton subunits that assemble to form two stacked rings. The two rings are formed by the ATPase domains (Huyton, T. et al., Struct. Biol. (2003) 144, 337-348; DeLaBarre, B. et al. Nat. Struct. Biol. (2003) 10, 856-863). The ‘top’ ring is formed by a hexamer of the D1 domains, whereas the ‘bottom’ ring is formed by a hexamer of the D2 domains. The N-domain extends outward from the D1 domain ring. Although it is clear that the D2 domain hydrolyzes ATP in vitro, the level of D1-specific ATPase activity reported by different investigators varies. Nevertheless, genetic studies in yeast suggest that ATP hydrolysis by both the D1 and D2 domains is essential for the function of p97 (Song, C. et al. J. Biol. Chem. (2003) 278, 3648-3655; Ye, Y. et al. J. Cell Biol. (2004) 162, 71-84). Binding of ATP to the D1 domain is also required for assembly of p97 (Wang, Q. et al. Biochem. Biophys. Res. Commun. (2003) 300, 253-260). Although ATP hydrolysis by the D2 domain is not required for assembly of p97 hexamer, it is thought that ATP hydrolysis by the D2 domain is a substrate conversion, resulting in their unfolding or dissociation from bound partners.

A prominent cellular function for p97 that has received considerable scrutiny is its role in the turnover of misfolded secretory proteins via the UPS (ubiquitin proteasome system). In this process, which is known as ERAD (for endoplasmic reticulum-associated degradation), proteins that fail to fold within the ER are retrotranslocated in a p97-dependent manner into the cytoplasm where they are degraded by the UPS (Ye, Y. et al. Nature (2004) 429, 841-847). In this process, p97 is thought to mediate extraction of substrates from the ER membrane. The complex p97 is also required for the turnover of cytosolic substrates of the UPS (Janiesch, P. C. et al. Nat. Cell Biol. (2007) 9, 379-390; Cao, K. et al. Cell (2003) 115, 355-367; Fu, X. et al. J. Cell Biol. (2003) 163, 21-26), although its role in turnover of cytosolic proteins is less understood.

The Valosin containing protein, p97, represents a suitable target for cancer therapeutics. The complex p97 and its function are essential for continued cellular viability, and so drugs that inhibit it should be antiproliferative. In other words, inhibition of p97 will cause undesirable protein concentration within the target cell. A consequential cellular reaction is often apoptosis or at least amelioration of cellular growth and mitosis. Also, p97 is known to be overproduced in multiple cancers (Yamamoto, S. et al. Ann. Surg. Oncol. (2005) 12, 925-934; Yamamoto, S. et al. Clin. Cancer Res. (2004) 10, 5558-5565; Yamamoto, S. et al. Ann. Surg. Oncol. (2004) 11, 697-704; Yamamoto, S. et al. Ann. Surg. Oncol. (2004) 11, 165-172) suggesting that its activity may be rate-limiting for the development of at least some cancers. p97 is known to be essential for ERAD (Carvalho, P. et al. Cell (2006) 126, 361-373), and recent studies suggest that cancer cells may be particularly dependent upon ERAD (Boelens, J. et al. In Vivo (2007) 21, 215-226). Furthermore, p97 has been linked to the turnover of IlcB and consequent activation of NF-kB (Dai, R. M. et al. J. Biol. Chem. (1998) 273, 3562-3573). NF-kB activity is important for the survival of some tumor cells, particularly in multiple myeloma (Keats, J. J. et. al. Cancer Cell (2007) 12, 131-144; Annunziata, C. M. et. al. Cancer Cell (2007) 12, 115-130). It has been suggested that bortezomib is active in multiple myeloma due to its ability to block turnover of proteins via the ERAD pathway and its ability to block turnover of IkB, thereby squelching the activity of NF-kB. Given that p97 is implicated in both ERAD and IlcB turnover but otherwise has a more restricted role in the UPS compared to the proteasome itself, drugs that target p97 may retain much of the efficacy of bortezomib but with less toxicity.

### GOALS OF THE INVENTION

There is a need to develop compounds suitable for inhibition of p97 activity and for methods of inhibiting the activity of p97 using such compounds. There is a need to develop such compounds for use in treatment of neoplastic malconditions.

### SUMMARY OF THE INVENTION

These and other needs are met by aspects of the present invention, one of which is directed to compounds having a tricyclic fused pyrimidine compound with a pyrimidine as the right aromatic ring of the tricyclic compound, a 5, 6 or 7 member saturated middle ring, and a 5, 6 or 7 member saturated left ring wherein the atoms at the fusion points between the middle and left rings are nitrogen or carbon and the left ring contains one heteroatom selected from nitrogen, oxygen or carbon. The tricyclic compound has two primary substituents and optionally more substituents bonded thereto. The two primary substituents are positioned at the P2 and P4 locations of the pyrimidine ring. In various embodiments, the P2 substituent is a 1- or 3-indole, a benzimidazole, a 1- or 3-benzopyrazole, a benzotriazole or an indane moiety. Preferably, the P2 substituent is an indole or benzimidazole. More preferably the P2 substituent is an indole moiety. Except for the benzotriazole and the benzopyrazole, the P2 moiety is preferably substituted at the 2 position with an alkyl or alkoxy group of 1 to 4 carbons. All versions of the P2 substituent are substituted at the 4 position with a polar, hydrogen bonding functional group as defined in the DEFINITIONS SECTION. Preferably this polar, hydrogen bonding functional group is a carboxylic acid, carboxamide, carboxylic ester, sulfonic acid, sulfonamide, sulfonic ester, aminomethyl, hydroxymethyl, boronic acid, boronic ester or alkyl versions thereof. In various embodiments, the P4 substituent is an arylalkyl amine moiety.

The tricyclic compound may also contain optional single or multiple aliphatic, functional and/or aromatic components as additional substituents positioned at various sites on the tricyclic compound as well as on the P2 and P4 groups.

In other aspects of the invention, the tricyclic fused pyrimidine compounds of the invention are formulated as pharmaceutical compositions and have an ability to inhibit Valosin containing protein p97 and to ameliorate, diminish, shrink, moderate and/or eliminate cells exhibiting neoplastic tendencies and/or abnormal function. In a further aspect of the invention, such compounds inhibit the ATPase activity of p97. Another aspect of the invention concerns treatment of malconditions and/or disease such as cancer through use of such compounds.

More specifically, an aspect of the invention is a compound having a tricyclic fused pyrimidine compound of Formula I.

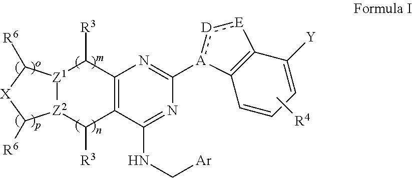

For Formula I, the symbols m, n, o, p, R¹-R⁷, A, D, E, X and Z are defined as follows.

The symbol m is an integer of 0, 1 or 2 and n is an integer of 0, 1 or 2. The symbols m and n designate the ring to which they are attached as the m/n ring and sum of m and n is 1, 2 or 3 so as to provide a 5, 6 or 7 member m/n ring with the bridge with the o/p ring at the top, middle or bottom of the m/n ring. Preferably the sum of m and n is 1 or 2 so as to provide a 5 or 6 member m/n ring; more preferably, the sum of m and n is 2 so as to provide a 6 member m/n ring.

The symbol o is an integer of 0, 1, 2 or 3 and the symbol p is an integer of 0, 1, 2 or 3. The symbols o and p designate the ring to which they are attached as the o/p ring and the sum of o and p is 2, 3 or 4 so as to provide a 5, 6 or 7 member ring. Preferably the sum of o and p is 1 or 2 so as to provide a 5 or 6 member o/p ring; more preferably, the sum of o and p is 2 so as to provide a 6 member o/p ring.

The symbols X and Z are defined as hetero or carbon atoms. The symbol X is NR¹, O or C(R¹)₂ and Z¹ and Z² are each independently selected from N or CR². A proviso applies to Z¹ and Z² such that when one of Z¹ and Z² is N, the other is CR². An additional proviso applies such that when one of Z¹ and Z² is N and X is NR¹ or O, then for the carbon in parenthesis that is adjacent to N of Z¹ or Z², the symbol o or p associated with this carbon is the integer 2 or 3. This proviso prevents substantially unstable configurations of X as oxygen or nitrogen and Z as nitrogen. If X and one of the Z's are both nitrogen and the number of carbons designated by one of the integers o or p is such that these nitrogens are bonded to each other, or are separated by a single carbon, the resulting structures are not fully stable. The moiety C—N—N—C can be enzymatically and/or hydrolytically cleaved and the moiety —N—C—N— is capable of functioning like a ketal and can be enzymatically or hydrolytically cleaved.

The R groups are defined independently and generally as aliphatic and/or functional groups according to the definitions of these groups provided in the DEFINITIONS SECTION. In particular, the group R¹ preferably is independently selected from hydrogen, a C¹ to C₄ straight or branched alkyl, or an acyl group of C₁ to C₄ carbons in length. Preferably each instance of R² and R⁴ is independently selected from hydrogen, a C₁ to C₄ straight or branched alkyl or halogen. Preferably each instance of R³ and R⁶ is independently selected from hydrogen, a C₁ to C₄ straight or branched alkyl, halogen or a double bond O or S. The double bond O or S means that each instance of R³ and R⁶ may be ═O or ═S.

Ar is a phenyl, fluorophenyl or a monocyclic five or six member aromatic ring optionally containing one or two heteroatoms each independently selected from O, N, or S; or a single substituent version thereof wherein the substituent is selected from halogen or C₁ to C₄ straight or branched alkyl. Preferably, Ar is phenyl or fluorophenyl.

The group at the 2 position of the pyrimidine ring is designated as the P2 group with the following formula I-A

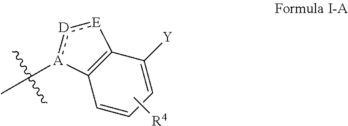

The A, D and E symbols of the P2 group are defined as hetero or carbon atoms while Y is defined as a polar, hydrogen bonding functional group and R⁴ is either absent or is defined as an aliphatic or functional group. In particular, A is N, C as a sp² carbon or CH as a sp³ carbon. D is N or CR⁵ as a sp² carbon. E is N, NR⁵ or CR⁵ as a sp² carbon. The dotted lines between A-D and D-E indicate a single or a double bond according to the identities of A, D and E such that when A is N, the bond between A and D is single and the bond between D and E is double. When A is C as a sp² carbon, the bond between A and D is double and the bond between D and E is single. When A is CH as a sp³ carbon, the bond between A and D is single and the bond between D and E is double. Each R⁵ is independently selected from hydrogen, C₁ to C₄ straight or branched alkyl, or C₁ to C₄ straight or branched alkoxy, provided that when D or E is NR⁵, R⁵ of NR⁵ is hydrogen or alkyl.

The symbol Y of P2 is a polar, hydrogen bonding functional group defined in the following DEFINITIONS section. In particular, Y is —CO₂H, —CO₂R′, —CONH₂, —CONR′₂, —NR′COR′, —SO₃H, —SO₂NR′₂, —NR′SO₂R′, —B(OH)₂, —B(OR′)₂, -tetrazolyl, —NR′₂, —CH₂NR′₂, —CN, —CH₂CN, —OH, —CH₂OR′, —CH₂CO₂H, —CH₂CO₂R′, —CH₂CONR′₂, —CH₂NR′COR′, —CH₂SO₂R′, —CH₂SO₃R′, —CH₂NR′SO₂R′ or —CH₂SO₂NR′₂. Each R′ is independently H or C₁ to C₄ straight or branched alkyl or phenyl or fluorophenyl, preferably H or alkyl, more preferably H, Me or Et; most preferably H or Me; especially most preferably H; and especially most preferably Me.

An additional aspect of the invention is directed to a pharmaceutical composition of a pharmaceutically acceptable carrier and the above described tricyclic compounds of Formula I, especially as set forth in the following Detailed Description.

Another aspect of the invention is directed to a method of decreasing Valosin containing protein (p97) activity or decreasing degradation of a proteasome system substrate, especially a ubiquitin substrate, by administration to a patient in need an effective therapeutic amount of the above described tricyclic compounds of Formula I.

Yet another aspect of the invention is directed to the treatment of neoplastic malconditions, cancer and other malconditions associated with p97 by administration to a patient in need the foregoing pharmaceutical composition.

### DETAILED DESCRIPTION OF THE INVENTION DEFINITIONS

Unless defined otherwise, all technical and scientific terms used herein have the same meaning as commonly understood by a person of ordinary skill in the art.

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

As used herein, “individual” (as in the subject of the treatment) or “patient” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “may” in the context of this application means “is permitted to” or “is able to” and is a synonym for the term “can.” The term “may” as used herein does not mean possibility or chance.

The term “disease” or “disorder” or “malcondition” are used interchangeably, and are used to refer to diseases or conditions wherein the p97 complex plays a role in the biochemical mechanisms involved in the disease or malcondition or symptom(s) thereof such that a therapeutically beneficial effect can be achieved by acting on the p97 complex. “Acting on” the p97 complex, or “modulating” the p97 complex, can include binding to the p97 complex and/or inhibiting the bioactivity of the p97 complex and/or allosterically regulating the bioactivity of the p97 complex in vivo.

The expression “effective amount”, when used to describe therapy to an individual suffering from a disorder, refers to the amount of a drug, pharmaceutical agent or compound of the invention that will elicit the biological or medical response of a cell, tissue, system, animal or human that is being sought, for instance, by a researcher or clinician. Such responses include but are not limited to amelioration, inhibition or other action on a disorder, malcondition, disease, infection or other issue with or in the individual's tissues wherein the disorder, malcondition, disease and the like is active, wherein such inhibition or other action occurs to an extent sufficient to produce a beneficial therapeutic effect. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. Recursive substituents are an intended aspect of the disclosed subject matter. One of ordinary skill in the art of medicinal and organic chemistry understands the versatility of such substituents. To the degree that recursive substituents are present in a claim of the disclosed subject matter, the total number should be determined as set forth above.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, less than the total number of carbon atoms in the universe and bounded by the understanding of the person of ordinary skill as to the size of the group as being reasonable for a molecular entity, and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded, as the number of atoms in the group cannot be infinite.

In general, “substituted” and “substituent” refer to an organic group as defined herein in which one or more bonds to a hydrogen atom contained therein are replaced by one or more bonds to a non-hydrogen atom. More particularly, the term “chemical substituent” refers to any and all aliphatic, aromatic and functional groups listed in this section that can be appended to an organic molecule. A functional group is an inorganic moiety such as halogen, sulfate, nitro, amino and the like as well as monocarbon functional groups such as carboxyl, carbonyl, carboxamide that are ordinary and typical optional substituents of organic molecules. In the context of this invention, recitation of this term without indication of specific groups constitutes the definition given above. Recitation of this term in combination with a Markush recitation of specific groups constitutes a subgenus of the understanding conveyed by the foregoing definition. The term “substituent” generally means any appropriate group named below that has an “yl”, “y” or “o” ending to designate that it is appended, attached or covalently bonded to another moiety such as but not limited to an aromatic framework. Examples include but are not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups.

Non-limiting examples of substituents J that can be bonded to a substituted carbon (or other) atom include F, Cl, Br, I, OR′, OC(O)N(R′)₂, B(OH)₂, B(OR′″)₂ with R′″ being C1 to C6 alkyl, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J. Preferably, each R′ independently is hydrogen or alkyl of 1 to 6 carbons, more preferably hydrogen or methyl or ethyl, most preferably hydrogen and R′ is not substituted by an additional J group.

In various embodiments, J can be halo, nitro, cyano, OR, NR₂, or R, or is C(O)OR, C(O)NR₂, OC(O)OR, OC(O)NR₂, N(R)C(O)OR, N(R)C(O)NR₂ or thio/thiono analogs thereof. By “thio/thiono analogs thereof”, with respect to a group containing an O, is meant that any or all O atoms in the group can be replaced by an S atom; e.g., for group C(O)OR, a “thio/thiono analog thereof” includes C(S)OR, C(O)SR, and C(S)SR; e.g., for group OC(O)NR₂, a “thio/thiono analog thereof” includes SC(O)NR₂, OC(S)NR₂, and SC(S)NR₂; and so forth. The symbol R independently in each instance is hydrogen or alkyl of 1 to 6 carbons, preferably hydrogen or methyl, more preferably hydrogen except that when J is R, R is alkyl of 1 to 4 carbons, preferably methyl or ethyl, more preferably methyl.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR′₂)ₙ wherein n is 1, 2, 3, or more, and each R′ is independently selected.

For all substituents, the first atom of the molecular formula of the substituent is the atom bonding the substituent to its corresponding moiety, e.g., for the functional group, N(R^{a})C(O)R^{a}, the N is bonded to the corresponding moiety substituted by this group. If the substituent is described in words, such as alkyenylamine, the phrase ending in “enyl” indicates the carbon atom bonding the substituent to its corresponding moiety. For substituents that display a single bonding site, such as carboxylic acid, sulfonic acid, fluoro, methyl and the like, the bonding arrangement is the expected arrangement.

“Aliphatic substituent, group or component” refers to any organic group that is non-aromatic. Included are acyclic and cyclic organic compounds composed of carbon, hydrogen and optionally of oxygen, nitrogen, sulfur and other heteroatoms. This term encompasses all of the following organic groups except the following defined aromatic and heteroaromatic groups. Examples of such groups include but are not limited to alkyl, alkenyl, alkynyl, corresponding groups with heteroatoms, cyclic analogs, heterocyclic analogs, branched and linear versions and such groups optionally substituted with functional groups, as these groups and others meeting this definition of “aliphatic” are defined below.

“Aromatic substituent, group or component” refers to any and all aromatic groups including but not limited to aryl, aralkyl, heteroalkylaryl, heteroalkylheteroaryl and heteroaryl groups. The term “aromatic” is general in that it encompasses all compounds containing aryl groups optionally substituted with functional groups (all carbon aromatic groups) and all compounds containing heteroaryl groups optionally substituted with functional groups (carbon-heteroatom aromatic groups), as these groups and others meeting this definition of “aromatic” are defined below.

As used herein, the term “optionally” means that the corresponding substituent or thing may or may not be present. It includes both possibilities.

“Alkyl” refers to a straight or branched or cyclic hydrocarbon chain radical consisting solely of carbon and hydrogen atoms, containing no unsaturation, having from one to ten carbon atoms (e.g., C₁-C₁₀ alkyl). Whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated. In some embodiments, it is a C₁-C₄ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, septyl, octyl, nonyl, decyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), 3-methylhexyl, 2-methylhexyl, and the like.

Unless stated otherwise specifically in the specification, an alkyl group is linear, branched or cyclic and has 1 to 6 carbons, preferably 1 to 4 carbons and is optionally substituted by one or more of substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂ where each R^{a} is independently hydrogen, alkyl, fluoroalkyl, carbocyclyl, carbocyclylalkyl, aryl, aralkyl, heterocyclyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Alkylaryl” refers to an -(alkyl)aryl radical where aryl and alkyl are as disclosed herein and which are optionally substituted by one or more of the substituents described as suitable substituents for aryl and alkyl respectively.

“Alkylhetaryl” refers to an -(alkyl)hetaryl radical where hetaryl and alkyl are as disclosed herein and which are optionally substituted by one or more of the substituents described as suitable substituents for aryl and alkyl respectively.

“Alkylheterocycloalkyl” refers to an -(alkyl) heterocycyl radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., C₂-C₁₀ alkenyl). Whenever it appears herein, a numerical range such as “2 to 10” refers to each integer in the given range; e.g., “2 to 10 carbon atoms” means that the alkenyl group may consist of 2 carbon atoms, 3 carbon atoms, etc., up to and including 10 carbon atoms. In certain embodiments, an alkenyl comprises two to eight carbon atoms. In other embodiments, an alkenyl comprises two to five carbon atoms (e.g., C₂-C₅ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

Unless stated otherwise specifically in the specification, an alkenyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Alkenyl-cycloalkyl” refers to an -(alkenyl)cycloalkyl radical where alkenyl and cyclo alkyl are as disclosed herein and which are optionally substituted by one or more of the substituents described as suitable substituents for alkenyl and cycloalkyl respectively.

“Carboxaldehyde” refers to a —(C═O)H radical.

“Carboxyl” refers to a —(C═O)OH radical.

“Cyano” refers to a —CN radical.

“Cycloalkyl” is a subcategory of alkyl and refers to a monocyclic or polycyclic radical that contains only carbon and hydrogen, and may be saturated, or partially unsaturated. Cycloalkyl groups include groups having from 3 to 10 ring atoms (i.e., C₂-C₁₀ cycloalkyl). Whenever it appears herein, a numerical range such as “3 to 10” refers to each integer in the given range; e.g., “3 to 10 carbon atoms” means that the cycloalkyl group may consist of 3 carbon atoms, etc., up to and including 10 carbon atoms. In some embodiments, it is a C₃-C₈ cycloalkyl radical. In some embodiments, it is a C₃-C₅ cycloalkyl radical. Illustrative examples of cycloalkyl groups include, but are not limited to the following moieties: cyclopropyl, cyclobutyl, cyclopentyl, cyclopentenyl, cyclohexyl, cyclohexenyl, cycloseptyl, cyclooctyl, cyclononyl, cyclodecyl, norbornyl, and the like.

Unless stated otherwise specifically in the specification, a cycloalkyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Cycloalkyl-alkenyl” refers to a -(cycloalkyl) alkenyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heterocycloalkyl” refers to a -(cycloalkyl) heterocycyl radical where cycloalkyl and heterocycloalkyl are as disclosed herein and which are optionally substituted by one or more of the substituents described as suitable substituents for heterocycloalkyl and cycloalkyl respectively.

“Cycloalkyl-heteroaryl” refers to a -(cycloalkyl) heteroaryl radical where cycloalkyl and heterocycloalkyl are as disclosed herein and which are optionally substituted by one or more of the substituents described as suitable substituents for heterocycloalkyl and cycloalkyl respectively.

“Alkoxy” refers to the group —O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched chain alkyls of from 1 to 4 carbon atoms.

“Substituted alkoxy” refers to alkoxy wherein the alkyl constituent is substituted (i.e., —O-(substituted alkyl)).

Unless stated otherwise specifically in the specification, the alkyl moiety of an alkoxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —C(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Alkoxycarbonyl” refers to a group of the formula (alkoxy) (C═O)— attached through the carbonyl carbon wherein the alkoxy group has the indicated number of carbon atoms. Thus a C₁-C₆ alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group. In some embodiments, C₁-C₄ alkoxy, is an alkoxy group which encompasses both straight and branched chain alkoxy groups of from 1 to 4 carbon atoms.

“Substituted alkoxycarbonyl” refers to the group (substituted alkyl)-O—C(O)— wherein the group is attached to the parent structure through the carbonyl functionality.

Unless stated otherwise specifically in the specification, the alkyl moiety of an alkoxycarbonyl group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Acyl” refers to the groups (alkyl)-C(O)—, (aryl)-C(O)—, (heteroaryl)-C(O)—, (heteroalkyl)-C(O)—, and (heterocycloalkyl)-C(O)—, wherein the group is attached to the parent structure through the carbonyl functionality. In some embodiments, it is a C₁-C₁₀ acyl radical which refers to the total number of chain or ring atoms of the alkyl, aryl, heteroaryl or heterocycloalkyl portion of the acyloxy group plus the carbonyl carbon of acyl, i.e. three other ring or chain atoms plus carbonyl. If the R radical is heteroaryl or heterocycloalkyl, the hetero ring or chain atoms contribute to the total number of chain or ring atoms.

Unless stated otherwise specifically in the specification, the “R” of an acyloxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Acyloxy” refers to a R(C═O)O— radical wherein “R” is alkyl, aryl, heteroaryl, heteroalkyl, or heterocycloalkyl, which are as described herein. In some embodiments, it is a C₁-C₄ acyloxy radical which refers to the total number of chain or ring atoms of the alkyl, aryl, heteroaryl or heterocycloalkyl portion of the acyloxy group plus the carbonyl carbon of acyl, i.e. three other ring or chain atoms plus carbonyl. If the R radical is heteroaryl or heterocycloalkyl, the hetero ring or chain atoms contribute to the total number of chain or ring atoms.

Unless stated otherwise specifically in the specification, the “R” of an acyloxy group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2-S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, unless stated otherwise specifically in the specification. When a —N(R^{a})₂ group has two Ra other than hydrogen they can be combined with the nitrogen atom to form a 4-, 5-, 6-, or 7-membered ring. For example, —N(R^{a})₂ is meant to include, but not be limited to, 1-pyrrolidinyl and 4-morpholinyl.

Unless stated otherwise specifically in the specification, an amino group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

“Amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. In some embodiments it is a C₁-C₄ amido or amide radical, which includes the amide carbonyl in the total number of carbons in the radical. The R₂ of —N(R)₂ of the amide may optionally be taken together with the nitrogen to which it is attached to form a 4-, 5-, 6-, or 7-membered ring. Unless stated otherwise specifically in the specification, an amido group is optionally substituted independently by one or more of the substituents as described herein for alkyl, cycloalkyl, aryl, heteroaryl, or heterocycloalkyl. An amide may be an amino acid or a peptide molecule attached to a compound of Formula (I), thereby forming a prodrug. Any amine, hydroxy, or carboxyl side chain on the compounds described herein can be amidified. The procedures and specific groups to make such amides are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

“Aryl” refers to a conjugated pi radical with six or ten ring atoms which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. The term includes polycyclic groups, i.e. rings which share adjacent pairs of ring atoms in other words, the fusion points of the two or more rings.

Unless stated otherwise specifically in the specification, an aryl moiety is optionally substituted by one or more substituents as defined above. Such substituents further are independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Aralkyl” or “arylalkyl” refers to an (aryl)alkyl- radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). Any amine, hydroxy, or carboxyl side chain on the compounds described herein can be esterified. The procedures and specific groups to make such esters are known to those of skill in the art and can readily be found in reference sources such as Greene and Wuts, Protective Groups in Organic Synthesis, 3rd Ed., John Wiley & Sons, New York, N.Y., 1999, which is incorporated herein by reference in its entirety.

Unless stated otherwise specifically in the specification, an ester group is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, heterocyclylalkyl, heteroaryl or heteroarylalkyl. Preferably each R^{a} independently is hydrogen or alkyl of 1 to 4 carbons, more preferably it is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Functional substituent, group or component” refers to a substituent capable of displaying functionality such as hydroxyl, ester, amide, amine, enamine, halogen, cyano, thio, oxidized sulfur, nitrogen or phosphorus groups, alkoxy, aldehyde, ketone, carboxylic acid, anhydride, urethane, urea, imine, amidine, hydroxylimine, hydroxylamine, nitrile, organometallic, and any other group capable of displaying dipole interaction and/or reactivity. See Basic Principles of Organic Chemistry, Roberts & Casario, W. A. Benjamin, publisher New York, N.Y. 1965, Chapter 10. Additional examples include hydroxy, halo, cyano, trifluoromethyl, trifluoromethoxy, nitro, trimethylsilanyl, —OR^{a}, —SR^{a}, —(CH₂)ₙ—SR^{a} (n is 1 or 2), —OC(O)—R^{a}, —N(R^{a})₂, —C(O)R^{a}, —B(OH)₂, —B(OR′)₂ —C(O)OR^{a}, —C(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), —R^{a}N(R^{a})₂ or —PO₃(R^{a})₂ and the chain extended variations thereof wherein the extension consists of —(CH₂)ₙ— (n is 1 or 2) is positioned between the above-described group and the moiety to which the functional group is bound so that for example —OR^{a} is chain extended as —(CH₂)ₙ—OR^{a}, or —C(O)N(R^{a})₂ is chain extended as —(CH₂)ₙ—C(O)N(R^{a})₂, and wherein each R^{a} is independently hydrogen, alkyl, fluoroalkyl, carbocyclyl, carbocyclylalkyl, aryl, aralkyl, heterocyclyl, heterocyclylalkyl, heteroaryl, heteroarylalkyl or any combination thereof. Preferably each R^{a} is independently hydrogen or linear, branched or cyclic alkyl of 1 to 6 carbons; more preferably it independently is hydrogen, methyl or ethyl; most preferably hydrogen; and R′ is C1-C6 alkyl, preferably methyl or ethyl, more preferably methyl.

A subcategory of the term “functional component” includes the foregoing groups that are polar and preferably are hydrogen bonding. The term “polar functional component” constitutes this subcategory and includes the foregoing examples except for olefinic groups and other non-polar groups. These non-polar groups are excluded from the term “polar functional component.”

The term“polar” means that the so designated group exhibits a dipole moment and/or significant electronegativity or electropositivity so that electromagnetic attraction between such polar groups occurs.

The term hydrogen bonding means that the group either will form a pseudobond with a polarized group containing hydrogen or is such a polarized group containing hydrogen.

“Halo”, “halide”, or, alternatively, “halogen” means fluoro, chloro, bromo or iodo. The terms “haloalkyl,” “haloalkenyl,” “haloalkynyl” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl” and “heteroalkenyl” are subcategories of alkyl and alkenyl and include optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given, e.g. C₁-C₄ heteroalkyl which refers to the chain length in total, which in this example is 4 atoms long. For example, a —CH₂OCH₂CH₃ radical is referred to as a “C₄” heteroalkyl, which includes the heteroatom center in the atom chain length description. Connection to the rest of the molecule may be through either a heteroatom or a carbon in the heteroalkyl chain.

A heteroalkyl group may be substituted with one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, heterocyclylalkyl, heteroaryl or heteroarylalkyl. Preferably each R^{a} independently is hydrogen or alkyl of 1 to 4 carbons, more preferably independently hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl respectively.

“Heteroalkylheterocycloalkyl” refers to an -(heteroalkyl)heterocycloalkyl radical where heteroalkyl and heteroaryl are as disclosed herein and which are optionally substituted by one or more of the substituents described as suitable substituents for heteroalkyl and heterocycloalkyl respectively.

“Heteroalkylcycloalkyl” refers to an -(heteroalkyl) cycloalkyl radical where heteroalkyl and cycloalkyl are as disclosed herein and which are optionally substituted by one or more of the substituents described as suitable substituents for heteroalkyl and cycloalkyl respectively.

“Heteroaryl” refers to a 5, 6 or 10-membered aromatic radical (e.g., C₅-C₁₃ heteroaryl) that includes one or more ring heteroatoms selected from nitrogen, oxygen and sulfur, and which may be a bicyclic, tricyclic or tetracyclic ring system. Whenever it appears herein, a numerical range refers to each integer in the given range. An N-containing “heteroaromatic” or “heteroaryl” moiety refers to an aromatic group in which at least one of the skeletal atoms of the ring is a nitrogen atom. The polycyclic heteroaryl group may be aromatic or non-aromatic, preferably aromatic. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, adeninyl, azabenzimidazolyl, azaindolyl, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, imidazopyridinyl, isoxazolopyridinyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thianaphthalenyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl), xanthinyl, guaninyl, quinoxalinyl, and quinazolinyl groups.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-(benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Preferred heteroaryl groups include pyridine, pyrimidine, piprazine, pyrazine, thiophene, furan, thiazaphene, imidazole and pyrrole.

Unless stated otherwise specifically in the specification, a heteraryl moiety is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, heterocyclylalkyl, heteroaryl or heteroarylalkyl. Preferably R^{a} is hydrogen or alkyl of 1 to 4 carbons, more preferably hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

Substituted heteroaryl also includes ring systems substituted with one or more oxide (—O—) substituents, such as pyridinyl N-oxides.

“Heterocyclic” refers to any or monocyclic or polycyclic moiety comprising at least one heteroatom selected from nitrogen, oxygen and sulfur. As used herein, heterocyclyl moieties can be aromatic or nonaromatic. The moieties heteroaryl and heterocyclyl alkyl are members of the heterocyclic group.

Unless stated otherwise, heterocyclic moieties are optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl, heterocyclylalkyl, heteroaryl or heteroarylalkyl. Preferably each R^{a} independently is hydrogen or alkyl of 1 to 4 carbons, more preferably it is independently hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Heteroarylalkyl” refers to a moiety having an aryl moiety, as described herein, connected to an alkylene moiety, as described herein, wherein the connection to the remainder of the molecule is through the alkylene group.

“Heterocyclylalkyl” refers to a stable 5, 6 or 10-membered non-aromatic ring radical having from one to six heteroatoms selected from nitrogen, oxygen and sulfur. Unless stated otherwise specifically in the specification, the heterocycloalkyl radical is a bicyclic, tricyclic or tetracyclic ring system, which may include bridged ring systems. The heteroatoms in the heterocycloalkyl radical may be optionally oxidized. One or more nitrogen atoms, if present, are optionally quaternized. The heterocycloalkyl radical is partially or fully saturated. The heterocycloalkyl may be attached to the rest of the molecule through any atom of the ring(s). Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

Unless stated otherwise specifically in the specification, a heterocycloalkyl moiety is optionally substituted by one or more substituents as defined above. Such substituents further independently include: alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —N(R^{a})C(O)R^{a}, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, where each R^{a} is independently hydrogen, alkyl, fluoroalkyl, carbocyclyl, carbocyclylalkyl, aryl, aralkyl, heterocycloalkyl, heteroaryl or heteroarylalkyl, heterocyclylalkyl, heteroaryl or heteroarylalkyl. Preferably each R^{a} independently is hydrogen or alkyl of 1 to 4 carbons, more preferably it independently is hydrogen or methyl, most preferably hydrogen. Each alkyl, alkenyl, alkynyl and other non-cyclo groups of the foregoing description contain 1 to 6 carbons, preferably 1 to 4 carbons and each cyclo group contains 3 to 6 carbons, preferably 5 or 6 carbons.

“Heterocyclylalkyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

“Sulfanyl” refers to the groups: —S-(optionally substituted alkyl), —S-(optionally substituted aryl), —S-(optionally substituted heteroaryl), and —S-(optionally substituted heterocycloalkyl).

“Sulfinyl” refers to the groups: —S(O)—H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocycloalkyl).

“Sulfonyl” refers to the groups: —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6-, or 7-membered ring. In some embodiments, it is a C₁-C₁₀ sulfonamido, wherein each R in sulfonamido contains 1 carbon, 2 carbons, 3 carbons, or 4 carbons total. A sulfonamido group is optionally substituted by one or more of the substituents described for alkyl, cycloalkyl, aryl, heteroaryl respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl respectively.

“Azido” refers to an N₃ group. An “azide” can be an organic azide or can be a salt of the azide (N₃⁻) anion. The term “nitro” refers to an NO₂ group bonded to an organic moiety. The term “nitroso” refers to an NO group bonded to an organic moiety. The term nitrate refers to an ONO₂ group bonded to an organic moiety or to a salt of the nitrate (NO₃⁻) anion.

“Urethane” (“carbamoyl” or “carbamyl”) includes N- and O-urethane groups, i.e., —NRC(O)OR and —OC(O)NR₂ groups, respectively.

“Sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

“Amidine” or “amidino” includes groups of the formula —C(NR)NR₂. Typically, an amidino group is —C(NH)NH₂.

“Guanidine” or “guanidino” includes groups of the formula —NRC(NR)NR₂. Typically, a guanidino group is —NHC(NH)NH₂.

A “salt” as is well known in the art includes an organic compound such as a carboxylic acid, a sulfonic acid, or an amine, in ionic form, in combination with a counterion. The tricyclic compounds of the invention may be such salts provided that appropriate salt forming moieties are present. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The tricyclic compounds of the invention may be hydrates when formulated in solid, preferably crystalline form. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. The tricyclic compounds of the invention may also be solvates in appropriate form such as solid, preferably crystalline form. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

A “prodrug” as is well known in the art is a substance that can be administered to a patient where the substance is converted in vivo by the action of biochemicals within the patient's body, such as enzymes, to the active pharmaceutical ingredient. Examples of prodrugs include esters of carboxylic acid groups, which can be hydrolyzed by endogenous esterases as are found in the bloodstream of humans and other mammals. Conventional procedures for the selection and preparation of suitable prodrug derivatives are described, for example, in “Design of Prodrugs”, ed. H. Bundgaard, Elsevier, 1985.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Additional descriptions of Markush groups include a series of atoms, groups or molecules ending with the penultimate term “or” and a series of atoms, groups or molecules introduced by the term “selected from.” Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzyhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzyhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

At various places in the present specification substituents of compounds of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, isopropyl, n-butyl, sec-butyl, isobutyl, etc. For a number qualified by the term “about”, a variance of 2%, 5%, 10% or even 20% is within the ambit of the qualified number.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

### Compounds

The invention is directed to compounds that inhibit ATPase Associated with a variety of Activities (AAA), the ATPase having the descriptive name Valosin containing protein, also known as p97, as well as methods to treat or prevent a disease or condition in a subject that would benefit by inhibition of p97. The compounds embodying of the invention incorporate a tricyclic ring structure with a right pyrimidine ring, a saturated middle penta, hexa or hepta m/n ring and a saturated left penta, hexa or hepta o/p ring. The tricyclic ring structure is substituted by aliphatic, functional and/or aromatic groups. Preferably, the tricyclic compound has as primary substituents of the pyrimidine ring an amino alkylaryl or aminoalkyl heteroaryl group that is substituted or unsubstituted at P4 position and a 5:6 bicyclic aromatic group at P2 position. More preferably, the tricyclic compound is substituted by a substituted or unsubstituted benzyl amine group at the P4 position and a mono, di or tri-nitrogen bicyclic 5:6 ring at the P2 position. The P2 substituent preferably is an indol-1-yl, an indol-3-yl, a benzopyrazol-1-yl, a benzopyrazol-3-yl, a benzimidazol-1-yl, a benzotriazol-1-yl or an indanyl ring. The tricyclic compound ring as well as the P2 and P4 groups may be substituted by multiple aliphatic, functional and/or aromatic groups described in the foregoing Definitions section. The aromatic 5:6 bicyclic P2 group may have hydrogen at the 2 position when the 2 position atom is carbon but preferably the substituent at this 2 position is a linear or branched alkyl group of 1 to 6 carbons, preferably 1 to 4 carbons, more preferably methyl or ethyl, most preferably methyl; or an alkoxy group of 1 to 6 carbons, preferably 1 to 4 carbons, more preferably methoxy or ethoxy, most preferably methoxy when the atom at the 2 position is carbon. Also preferably, the aromatic 5:6 bicyclic P2 group is substituted at the 4 position by a polar, hydrogen bonding functional group. The substituent at the 4 position is an aspect of the biological functionality of the tricyclic compounds of the invention which enhances the inhibition of the p97 enzyme complex.

A preferred embodiment of the tricyclic fused pyrimidine compound of the invention is a tricyclic compound of Formula I:

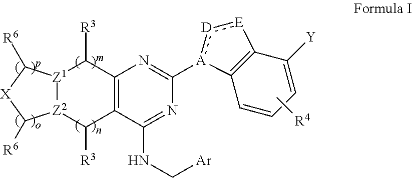

The variable symbols m, n, o, p, A, D, E, R¹-R⁶, R′, X, Y, and Z are as defined in the Summary of the Invention. The generic descriptions of R¹ through R⁶ and R′ are given above in the Summary of the Invention and are repeated here. The subgeneric, preferred, more preferred and especially preferred descriptions of these substituents are given in the following Detailed Description. The degrees of preference for the substituents are repeated in the Claims.

The tricyclic compounds of the invention have the m/n ring and the o/p rings each independently configured as a saturated pentacyclic, hexacyclic or heptacyclic ring with the Z¹-Z² fusion joining the m/n and o/p rings being C—C, C—N or N—C. The tricyclic core of this configuration is represented by Formula I-B:

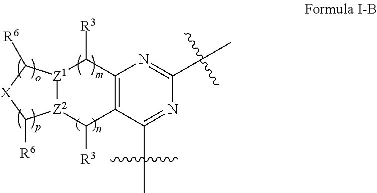

illustrations of the 5:6:Py and 6:6:Py cores of Formula I (Py is the pyridine ring) with designation of Z¹-Z² but without designation of P2, of P4 and of R¹ to R⁶ is given in Table I, while X is not designated in cores 1-18 but is designated in cores 19-33. In this illustration R³ and R⁶ are not shown, nevertheless they are to be regarded as present as designated by Formula I, all text concerning Formula I and the subgeneric and preferred versions of R³ and R⁶. In addition, X is not shown in cores 1-18 but is nevertheless to be regarded as present as designated by Formula I all text concerning Formula I and the subgeneric and preferred versions of X. Examples of the atoms X, Z¹ and Z² are shown in cores 19-33. These configurations of X, Z¹ and Z² in cores 19-33 are preferred. Accordingly, for cores 1-18, X would appear as any one of the non-fusion atoms of the o/p ring while examples of X are shown in cores 19-33. For all cores 1-33, the substituents R³ and R⁶ would also appear at the designated positions according to Formula I. In addition, the substituents R¹, R², R⁴, R⁵, the P2 group with its Y substituent and the P4 group NH—CH₂—Ar are to be regarded as present in all cores 1-33 as designated by Formula I, all text concerning Formula I and the subgeneric and preferred versions of these groups and substituents.

TABLE I

| | |
|---|---|
| 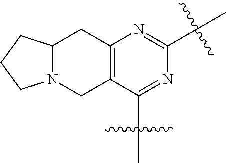 [SMILES: *c1nc(*)c2c(n1)CC1CCCN1C2] | Core-1 |
| 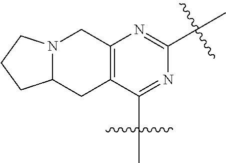 [SMILES: *c1nc(*)c2c(n1)CN1CCCC1C2] | Core-2 |
| 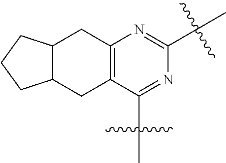 [SMILES: *c1nc(*)c2c(n1)CC1CCCC1C2] | Core-3 |
| 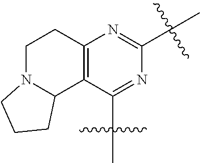 [SMILES: *c1nc(*)c2c(n1)CCN1CCCC21] | Core-4 |
| 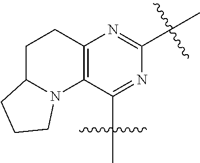 [SMILES: *c1nc(*)c2c(n1)CCC1CCCN21] | Core-5 |
| 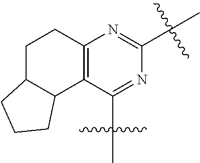 [SMILES: *c1nc(*)c2c(n1)CCC1CCCC21] | Core-6 |
| 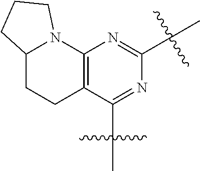 [SMILES: *c1nc(*)c2c(n1)N1CCCC1CC2] | Core-7 |
| 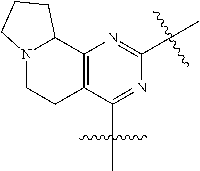 [SMILES: *c1nc(*)c2c(n1)C1CCCN1CC2] | Core-8 |
| 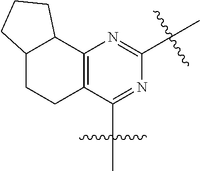 [SMILES: *c1nc(*)c2c(n1)C1CCCC1CC2] | Core-9 |
| 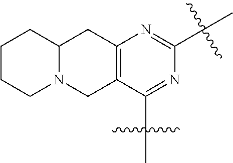 [SMILES: *c1nc(*)c2c(n1)CC1CCCCN1C2] | Core-10 |
| 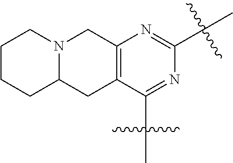 [SMILES: *c1nc(*)c2c(n1)CN1CCCCC1C2] | Core-11 |
| 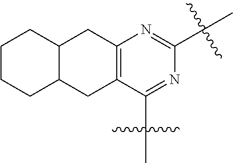 [SMILES: *c1nc(*)c2c(n1)CC1CCCCC1C2] | Core-12 |
| 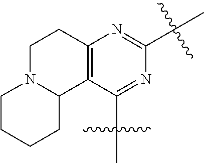 [SMILES: *c1nc(*)c2c(n1)CCN1CCCCC21] | Core-13 |
| 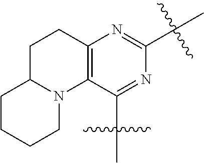 [SMILES: *c1nc(*)c2c(n1)CCC1CCCCN21] | Core-14 |
| 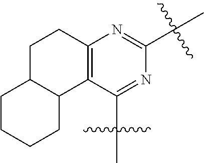 [SMILES: *c1nc(*)c2c(n1)CCC1CCCCC21] | Core-15 |
| 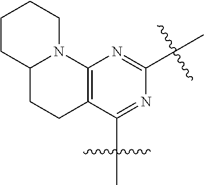 [SMILES: *c1nc(*)c2c(n1)N1CCCCC1CC2] | Core-16 |
| 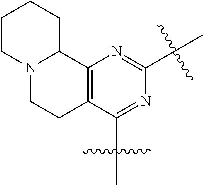 [SMILES: *c1nc(*)c2c(n1)C1CCCCN1CC2] | Core-17 |
| 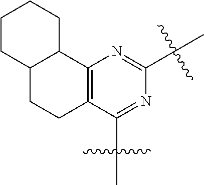 [SMILES: *c1nc(*)c2c(n1)C1CCCCC1CC2] | Core-18 |
| 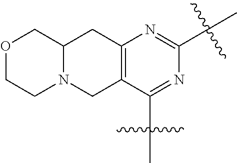 [SMILES: *c1nc(*)c2c(n1)CC1COCCN1C2] | Core-19 |
| 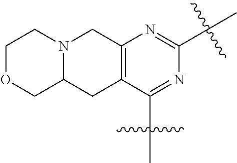 [SMILES: *c1nc(*)c2c(n1)CN1CCOCC1C2] | Core-20 |
| 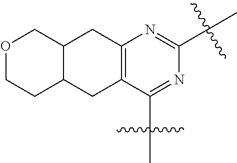 [SMILES: *c1nc(*)c2c(n1)CC1COCCC1C2] | Core-21 |
| 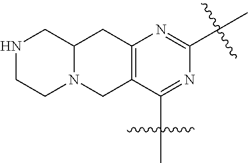 [SMILES: *c1nc(*)c2c(n1)CC1CNCCN1C2] | Core-22 |
| 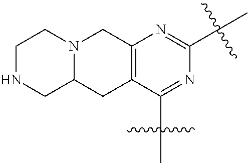 [SMILES: *c1nc(*)c2c(n1)CN1CCNCC1C2] | Core-23 |
| 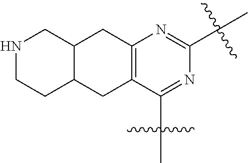 [SMILES: *c1nc(*)c2c(n1)CC1CNCCC1C2] | Core-24 |
| 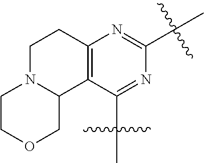 [SMILES: *c1nc(*)c2c(n1)CCN1CCOCC21] | Core-25 |
| 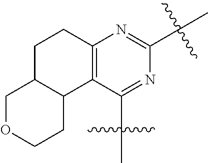 [SMILES: *c1nc(*)c2c(n1)CCC1COCCC21] | Core-26 |
| 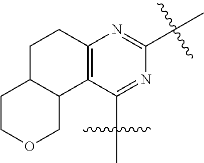 [SMILES: *c1nc(*)c2c(n1)CCC1CCOCC21] | Core-27 |
| 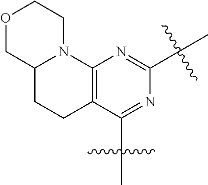 [SMILES: *c1nc(*)c2c(n1)N1CCOCC1CC2] | Core-28 |
| 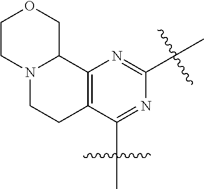 [SMILES: *c1nc(*)c2c(n1)C1COCCN1CC2] | Core-29 |
| 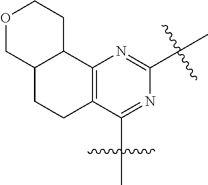 [SMILES: *c1nc(*)c2c(n1)C1CCOCC1CC2] | Core-30 |
| 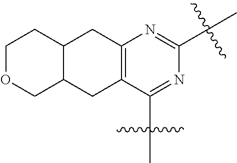 [SMILES: *c1nc(*)c2c(n1)CC1CCOCC1C2] | Core-31 |
| 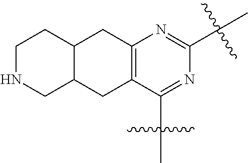 [SMILES: *c1nc(*)c2c(n1)CC1CCNCC1C2] | Core-32 |
| 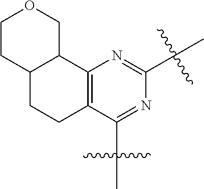 [SMILES: *c1nc(*)c2c(n1)C1COCCC1CC2] | Core-33 |

The cores for 5:5:Py, 5:7:Py, 6:5:Py, 6:7:Py, 7:5:Py, 7:6:Py and 7:7:Py with the symbols X, Z¹, Z² and R¹ through R⁶ at the positions designated by Formula I are similarly arranged. For example, the 5:5:Py core would have two arrangements of the o/p:m/n rings with each arrangement having three variations for Z¹-Z² and the 7:7:Py core would have four arrangements for the o/p:m/n rings with each arrangement having three variations for Z¹-Z².

Embodiments of the V:6:Py and V:5:Py tricyclic compounds of the invention in which the symbol V represents the o/p penta, hexa and hepta ring include Formulas II, III, IV, V and VI. The symbols A, D, E, o, p, X, Y, Z¹, Z², and R¹ through R⁶ and R′ have the same Markush and functional definitions, generic descriptions, substituent specific descriptions, preferred descriptions and alternative descriptions the same as recited for Formula I.

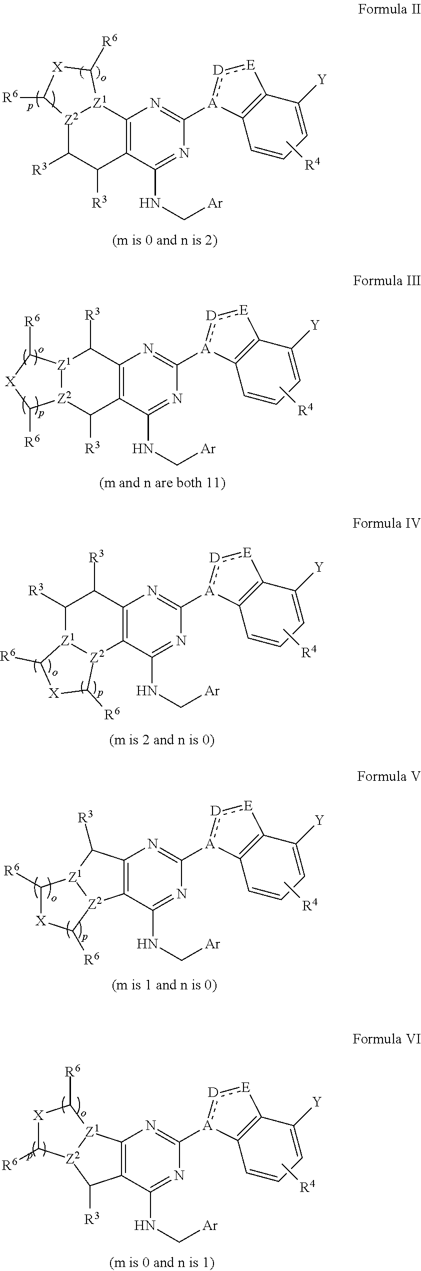

The P2 group is a 5:6 hetero-aromatic group of any of the formulas P2-A through P2-G wherein Y, R⁴ and R⁵ have the definitions given in the Summary and in the following text. The range of choices for symbols A, D and E produce formulas P2-A through P2-G. The embodiments P2E and P2F have tautomeric forms involving the allyl and vinyl amine moieties of the five member rings of P2E and P2F (when R⁵ of P2F is hydrogen).

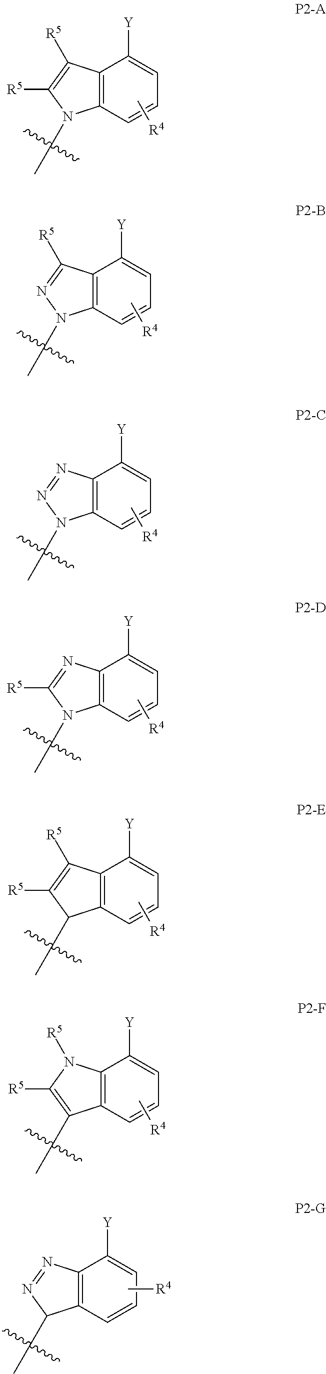

Preferred P2 groups include P2-A, P2-B, P2-C, P2-D and P2-F; more preferred P2 groups include P2-A, P2-B, P2-D and P2-F; most preferred P2 groups include P2-A and P2-D. An especially preferred P2 group is P2-A. Another especially preferred P2 group is P2-D. A further preferred P2 group is P2-B. Yet another preferred P2 group is P2-F. In all of these designated P2 groups, the substituents Y, R⁴ and R⁵ include the Markush groups recited as general disclosures, recited as preferred disclosures, recited as more preferred disclosures and recited as alternative disclosures. Any combination of these Markush groups is included so that by way of example, a general disclosure of Y combined with an alternative or especially preferred disclosure of R⁴ is included.

The Y substituent is a small to moderate sized, polar, hydrogen bonding functional group as defined in the DEFINITIONS section. The Y substituent of this P2 group significantly enhances the ability of the tricyclic compounds of the invention to inhibit the protein-ubiquitin cleavage by the p97 enzyme complex.

Embodiments of the Y substituent include hydroxyl, ester, amide, amine, enamine, cyano, thio, oxidized sulfur, nitrogen or phosphorus groups, alkoxy, aldehyde, ketone, carboxylic acid, anhydride, urethane, urea, imine, amidine, hydroxylimine, hydroxylamine, nitrile, organometallic, and any other group capable of displaying dipole interaction and/or reactivity such as is described in Basic Principles of Organic Chemistry. Roberts & Casario, W. A. Benjamin, publisher New York, N.Y. 1965, Chapter 10.

Additional embodiments of the Y substituent include hydroxy, cyano, nitro, trimethylsilanyl, —OR^{a}, —SR^{a}, —(CH₂)ₙ—SR^{a} (n is 1 or 2), —OC(O)—R^{a}, —N(R^{a})₂, —C(O)R^{a}, —B(OH)₂, —B(OR′)₂, —C(O)OR^{a}, —C(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), —R^{a}N(R^{a})₂ or —PO₃(R^{a})₂.

Further embodiments of the Y substituent include the chain extended variations thereof wherein the extension consists of —(CH₂)ₙ— (n is 1 or 2) and is positioned between the above-described embodiments of Y and the moiety to which this functional group Y is bound so that for example —OR^{a} is chain extended as —(CH₂)ₙ—OR^{a}, and —C(O)N(R^{a})₂ is chain extended as —(CH₂)ₙ—C(O)N(R^{a})₂.

In each of these embodiments of Y, each R^{a} is independently hydrogen, alkyl, fluoroalkyl, carbocyclyl, carbocyclylalkyl, aryl, aralkyl, heterocyclyl, heterocyclylalkyl, heteroaryl, heteroarylalkyl or any combination thereof wherein each alkyl group independently is a linear or branched C1 to C6, preferably C1 to C4 group; the carbocyclyl group is a cyclic alkyl group of 3 to 6 carbons, the carbocyclylalkyl group is a cyclic alkyl group of the same number of carbons bonded to a linear or branched alkyl group of 1 to 4 carbons, the aryl group is a C6 or C10 aryl group, the heterocyclyl group is the same as a carbocyclyl group except that it contains one or two heteroatoms selected from N, O or S; the heterocyclylalkyl group is the same as the heterocyclyl group and is bonded to a linear or branched alkyl group of 1 to 4 carbons, the heteroaryl group is a 6 or 10 membered aromatic group containing 1, 2 or 3 heteroatoms each independently selected from N, O or S, and the heteroarylalkyl group is the same as the heteroaryl group and is bonded to a linear or branched alkyl group of 1 to 4 carbons.

Preferably each R^{a} is independently hydrogen or linear, branched or cyclic alkyl of 1 to 6 carbons. More preferably R^{a} independently is hydrogen, methyl or ethyl; most preferably hydrogen.

Y may preferably be a functional substitutent selected from the group consisting of B(OH)₂, B(OR)₂ wherein R is an alkyl group of 1 to 6 carbons, OR^{d}, (CH₂)ₙOR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d}, —N(R^{d})C(O)N(R^{d})₂, N(R^{d})C(NR^{d})N(R^{d})₂, N(R^{d})S(O)ₜR^{d}, S(O)ₜOR^{d}, S(Oₜ)R^{d}, S(O)ₜN(R^{d})₂, N(R^{d})₂, (CH₂)ₙN(R^{d})₂, PO₃(R^{d})₂, and C(O)R^{d}. Each n is independently an integer of 1, 2 or 3, preferably 1. Each t is independently an integer of 1 or 2, preferably 2. Each R^{d} is independently hydrogen, alkyl of 1 to 6 carbons, fluoroalkyl of 1 to 6 carbons, carbocyclyl of 3 to 10 carbons, carbocyclylalkyl of 4 to 12 carbons, aryl of 6 to 10 carbons, aralkyl, heterocyclyl, heterocyclylalkyl, heteroaryl, heteroarylalkyl, alkenyl of 2 to 6 carbons, alkynyl or 2 to 6 carbons or any combination thereof wherein each of these R^{d} groups is defined as given for the foregoing R^{a} group. Preferably, R^{d} is hydrogen, phenyl or alkyl of 1 to 6 carbons, preferably 1 to 3 carbons, more preferably methyl or ethyl. More preferably, R^{d} is hydrogen, methyl or ethyl. Most preferably, R^{d} is hydrogen or methyl.

More preferred embodiments of Y include —CO₂H, —CO₂R′, —CONH₂, —CONR′₂, —NR′COR′, —SO₃H, —SO₂NR′₂, —NR′SO₂R′, —B(OH)₂, —B(OR′)₂, -tetrazolyl, —NR′₂, —CH₂NR′₂, —CN, —CH₂CN, —OH, —CH₂OR′, —CH₂CO₂H, —CH₂CO₂R′, —CH₂CONR′₂, —CH₂NR′COR′, —CH₂SO₂R′, —CH₂SO₃R′, —CH₂NR′SO₂R′ or —CH₂SO₂NR′₂. Each R′ is independently H or C₁ to C₄ straight or branched alkyl.

Y is also more preferably selected from the group consisting of boronic acid, boronic ester with the ester group being straight, branched or cyclic alkyl of 1 to 6 carbons, carboxylic acid, carboxyl ester with the ester group being straight, branched or cyclic alkyl of 1 to 6 carbons, carboxamide, N-alkyl carboxamide of 1 to 6 carbons in the straight, branched or cyclic alkyl group, sulfonic acid, sulfonyl ester with the ester group being straight, branched or cyclic alkyl of 1 to 6 carbons, sulfonamide, alkyl substituted sulfonamide with the alkyl group being straight, branched or cyclic of 1 to 6 carbons, amine (NH₂), mono or dialkyl amine with the alkyl being straight, branched or cyclic of 1 to 6 carbons, N-alkyl amino methyl with the alkyl group being straight, branched or cyclic of 1 to 6 carbons, nitrile, hydroxyl, straight, branched or cyclic hydroxy alkyl of 1 to 6 carbons in the alkyl group, or straight, branched or cyclic alkoxy of 1 to 6 carbons.

Especially more preferable embodiments of Y include —CO₂H, —CO₂R′, —CONH₂, —CONR′₂, —NR′COR′, —SO₃H, —SO₂NR′₂, —NR′SO₂R′, —B(OH)₂, —B(OR′)₂, -tetrazolyl, —NR′₂, —CH₂NR′₂, and —CN. Each R′ is defined as H or C1-C6 alkyl, preferably hydrogen, methyl or ethyl, more preferably methyl and in the alternative, more preferably hydrogen.

Particularly more preferable embodiments of Y include —CONH₂, -tetrazolyl, —SO₂NH₂, —B(OH)₂ and —CH₂NH₂.

Most preferably embodiments of Y include —CONH₂ and —SO₂NH₂

The position of Y at the 4 position of the P2 group is an aspect for improved development of the inhibition activity of the tricyclic fused pyrimidine compounds of Formula I against the p97 enzyme complex. Any polar or lipophilic group at this position confers higher activity than does hydrogen at this position. The presence of a polar, hydrogen bonding group such as an boronic acid, carboxamide, carboxylic acid, sulfonamide, sulfonic acid, hydroxyl, alkylenyl alcohol (eg., CH₂OH and similar substituents), amine or alkylenyl amine (eg., CH₂NH₂) confers higher activity than does a lipophilic (non-polar) group such as methyl or ethyl.

Although it is not a condition of the invention, it is believed that the presence of an accessible polar hydrogen bonding group at the Y position facilitates competitive or non-competitive binding with the enzymatic site or allosteric binding with S and R moieties adjacent to the enzymatic site of p97 and thereby promotes inhibition of enzymatic activity.

The Ar group is phenyl, fluorophenyl, aminocarbonylphenyl or a monocyclic five or six member aromatic ring optionally containing one or two heteroatoms selected from N, O and/or S. Embodiments of the Ar group include phenyl, thiophenyl, pyrrolyl, furanyl, imidazolyl, oxazolyl, thiazolyl, pyridinyl and pyrimidinyl. A preferred group for Ar is phenyl or substituted phenyl wherein the substituent is fluoro, trifluoromethyl, boronic acid, boronic alkyl ester with a C1 to C6 alkyl, carboxylic acid, carboxylic alkyl ester with a C1 to C6 alkyl, carboxamide, sulfonic acid, sulfonamide. The preferred substituent is fluoro, boronic acid, boronic ester, carboxylic acid, carboxamide, sulfonic acid, sulfonic ester. The more preferred substituent is fluoro, boronic acid, carboxylic acid, sulfonic acid. The most preferred substituent is fluoro or boronic acid. The most preferred Ar group is phenyl or p-fluorophenyl.

The substituents R¹, R³, R⁴, R⁵ and R⁶ are aliphatic and/or functional substituents for the embodiments as provided in Formula I and the general, preferred, more preferred and especially more preferred definitions thereof.

The P2 substituent R⁴ is preferably absent from the benzo group of P2 such that except for Y, the non-fusion point carbons of the benzo group are all C—H. In the alternative, R⁴ may be selected from the group consisting of linear, branched or cyclic alkyl or alkenyl of 1 to 6 carbons (2 minimum for alkenyl), halogen, B(OH)₂, B(OR)₂ with 1 to 6 carbons in the R group, OR^{d}, CN, SR^{d}, OC(O)R^{d}, C(O)R^{d}, C(O)OR^{d}, OC(O)N(R^{d})₂, C(O)N(R^{d})₂, N(R^{d})C(O)OR^{d}, N(R^{d})C(O)R^{d}, —N(R^{d})C(O)N(R^{d})₂, N(R^{d})C(NR^{d})N(R^{d})₂, N(R^{d})S(O)ₜR^{d}, S(O)ₜOR^{d}, S(O)ₜN(R^{d})₂, N(R^{d})₂, (CH₂)_{q}N(R^{d})₂ and PO₃(R^{d})₂ wherein each R^{d} is independently hydrogen, alkyl, fluoroalkyl, carbocyclyl, carbocyclylalkyl, aryl, aralkyl, heterocyclyl, heterocyclylalkyl, heteroaryl, heteroarylalkyl alkenyl, alkynyl or any combination thereof. Each t is independently selected from the group of integers of 1 and 2. Each q is independently an integer of 0, 1, 2 or 3, n is an integer of 0, 1 or 2, preferably 1, more preferably 0. Preferably, R^{d} is hydrogen, 1 or alkyl of 1 to 6 carbons, preferably 1 to 3 carbons, more preferably methyl or ethyl. More preferably, R^{d} is hydrogen, methyl or ethyl. Most preferably, R^{d} is hydrogen or methyl.

Each R¹ substituent of the N and C designations for X is independently selected from hydrogen, straight, branched or cyclic alkyl of 1 to 6 carbons, preferably linear or branched alkyl of 1 to 4 carbons, more preferably methyl or ethyl, acyl of 1 to 4 carbons including formyl, acetyl, propionyl or butyryl, carboxylic acid, carboxyl ester with the ester group being straight, branched or cyclic alkyl of 1 to 6 carbons, N-alkyl amino methyl with the alkyl group being straight, branched or cyclic of 1 to 6 carbons, boronic acid, sulfonic acid, boronic ester with the ester groups each independently being straight, branched or cyclic alkyl of 1 to 6 carbons, sulfonyl ester with the ester group being straight, branched or cyclic alkyl of 1 to 6 carbons, sulfonamide, amine (NH₂), mono, di or trialkyl amine with the alkyl being straight, branched or cyclic of 1 to 6 carbons (applies only when X is C), nitrile, carboxamide, N-alkyl carboxamide of 1 to 6 carbons in the straight, branched or cyclic alkyl group, perfluoroalkyl of 1 to 3 carbons, or straight, branched or cyclic alkoxy of 1 to 6 carbons. Preferably, R¹ is hydrogen, methyl, ethyl, formyl, acetyl, propionyl or carboxamide (CONH₂). More preferably, R¹ is hydrogen or methyl, most preferably hydrogen.

R² is selected from hydrogen, halogen (preferably fluoro, chloro or bromo, more preferably fluoro or chloro, most preferably fluoro), straight, branched or cyclic alkyl of 1 to 6 carbons, preferably linear or branched alkyl of 1 to 4 carbons, more preferably methyl or ethyl. Preferably, each R² independently is hydrogen or methyl, more preferably hydrogen.

Each instance of R³ and R⁶ is independently selected from the group consisting of hydrogen and linear, branched or cyclic alkyl or alkenyl of 1 to 6 carbons (2 minimum for alkenyl). More preferably, each of these groups may be each independently selected from the group consisting of hydrogen and an alkyl group of 1 to 3 carbons. Most preferably, each of these groups may be hydrogen or methyl. Alternatively, one or two of R³ and R⁶ is a keto or thioketo group. Preferably, one instance of R³ and R⁶ is a keto or thioketo group, preferably a keto group. An example of the keto alternative for R³ shown as the keto group on Core 34

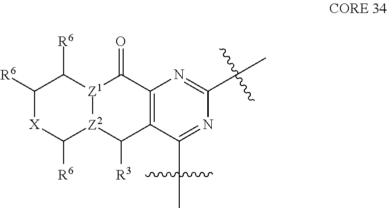

The substituent R⁵ is hydrogen, linear or branched alkyl of 1 to 6 carbons or linear or branched alkoxy of 1 to 6 carbons, preferably hydrogen, linear or branched alkyl of 1 to 4 carbons or linear or branched alkoxy of 1 to 4 carbons, more preferably hydrogen, methyl, ethyl, methoxy or ethoxy; most preferably hydrogen, methyl or methoxy; especially most preferably methyl or methoxy.

More preferred embodiments of the V:6:PY tricyclic compounds of Formula I include the tricyclic compounds of Formulas II, III and IV with the substituents Y and R¹-R⁶ designated as follows. Y is selected from —COOH, —COOR with R being alkyl of 1 to 3 carbons, —B(OH)₂, —B(OR)₂ with R being alkyl of 1 to 3 carbons; —CN, —Vₐ(CH₂)_{b}W, —N(R^{a})₂, —CO(NR^{a})₂—SO₂R^{a}, —SO₂N(R^{a})₂. The integer designator b is 0, 1, 2 or 3, preferably 1 or 0. V for these preferred embodiments is O, S, NR^{a}, CO₂, CO, CONH, NHCO and N-alkyl. The integer designator a is 0 or 1. W for these preferred embodiments is amine, alkylamine, alkoxy, alkonyloxy, carboxylic acid, carboxamide, aminocarbonylalkyl, carboxyl ester or N-alkyl carboxamide, sulfonic acid, sulfonamide, boronic acid or boronic alkyl ester. R^{a} of this more preferred embodiment of Y is H, Me, Et, preferably H, and when two R^{a}'s are present each is selected independently. The preferred selections for R⁴ include hydrogen, methyl, ethyl, n-propyl, isopropyl, n-butyl, isobutyl. More preferred selections for R⁴ include hydrogen, methyl, ethyl, n-propyl, methoxy, ethoxy, carboxamide, sulfonamide and especially hydrogen. The preferred selections for each independently selected R¹ include hydrogen, methyl, ethyl, formyl and acetyl. The preferred selections for each of R², R³, and R⁶ independently include hydrogen, methyl and ethyl, especially hydrogen. Preferred selections for R⁵ include hydrogen, methyl, ethyl when D and/or E is C or N, and when D and/or E is C, include also methoxy and ethoxy. Especially preferred selections for R⁵ include methyl or ethyl for D and/or E as N or C and also include methoxy or ethoxy for D and/or E as C. Methyl and methoxy are preferred for R⁵ when D and/or E os C and hydrogen is preferred for R⁵ when D and/or E is N.

Most preferred substituent embodiments (Y and the R groups) of the invention include the the tricyclic compounds of Formula I where Y is B(OH)₂, B(OMe or OEt)₂, SO₃H, SO₂NH², NHSO₂CH₃, COOH, COOMe, COOEt, CONH₂, NHCOCH₃, CN, CH₂NH₂, CH₂NHCOCH₃, CH₂NHSO₂CH₃, CH₂OH, CH₂CH₂OH or OH. R⁴ is H, Me, Et, CONH₂, SO₂NH₂, B(OH)₂, B(OMe or OET)₂, OMe, OEt, CN, F, Cl or Br, most especially, H or Me and of these two substituents, preferably H. R¹, R², R³, R⁵ and R⁶ are each independently H, methyl or ethyl, more preferably H or methyl. Additionally, R¹ is preferably acetyl. Additionally R⁵ preferably is also methoxy or ethoxy when D and/or E is C. Most preferably, only one B(OH)₂, B(OMe or OEt)₂ is present on Formula I when B(OH)₂, B(OMe or OEt)₂ is chosen.

The number of boronic acid or boronic ester groups as substituents anywhere on Formula I is one.

In the foregoing descriptions of Y, the R's and Ar substituents, unless a particular paragraph groups together several of these substituents, each individual description of a substituent can be combined with any other individual description of a substituent. For example, Ar as phenyl or fluorophenyl can be grouped with a Markush group for Y that is the generic functional description of Y or is a description of Y listing specific functional groups, or is a preferred description of Y listing a selection of specific functional groups. The same logic applies to the combination of Y and R⁴ for example.

### Preferred Subgeneric Embodiments of the Tricyclic Pyrimidine Compounds

Subgeneric embodiments of the tricyclic compounds of Formula I include the designations provided in the following specifications for the tricyclic compound variables.

These subgeneric tricyclic compounds are arranged according to the identity of the P2 group. Within each category of P2 group, the compounds are arranged according to the X and Z designations and the P2 bicyclic substituent. In this arrangement, a succeeding embodiment includes the substituent specifications of any one or more of the preceeding embodiments as well as standing as an independent designation for Formula I. For example, this means that embodiment 5 specifies substituents of Formula I and as alternatives includes the embodiments of subgeneric embodiment 1, in other words; 5 combined with 1; 5 combined with 2; 5 combined with 3; and 5 combined with 4. Embodiment 5 also can be combined with multiple preceeding embodiments, for example: 5 combined with 1 and 2; 5 combined with 1 and 3; 5 combined with 1 and 4; 5 combined with 2 and 3; 5 combined with 2 and 4; and lastly, 5 combined with 3 and 4. This arrangement applies to alternatives 1-24.
1) A tricyclic fused pyrimidine compound of Formula I wherein each instance of R³ and R⁶ is independently selected from hydrogen, branched or straight alkyl or halogen.
2) A tricyclic fused pyrimidine compound of Formula I wherein each instance of R³ and R⁶ is independently selected from hydrogen or branched or straight alkyl.
3) A tricyclic fused pyrimidine compound of Formula I or Formula II wherein R³ and R⁶ are both hydrogen, Z¹ is N and Z² is CH, or Z¹ is CH and Z² is N, or Z¹ and Z² are both CH.
4) A tricyclic fused pyrimidine compound of Formula I or III wherein R³ and R⁶ are both hydrogen, Z¹ is N and Z² is CH, or Z¹ is CH and Z² is N, or Z¹ and Z² are both CH.
5) A tricyclic fused pyrimidine compound of Formula I or IV, wherein R³ and R⁶ are both hydrogen, Z¹ is N and Z² is CH, or Z¹ is CH and Z² is N, or Z¹ and Z² are both CH.
6) A tricyclic fused pyrimidine compound of Formula I or V, wherein R³ and R⁶ are both hydrogen, Z¹ is N and Z² is CH, or Z¹ is CH and Z² is N, or Z¹ and Z² are both CH.
7) A tricyclic fused pyrimidine compound of Formula I or VI wherein R³ and R⁶ are both hydrogen, Z¹ is N and Z² is CH, or Z¹ is CH and Z² is N, or Z¹ and Z² are both CH.
8) A tricyclic fused pyrimidine compound of Formula I or any one or more of the foregoing embodiments wherein Z¹ and Z² are both CH.
9) A tricyclic fused pyrimidine compound of Formula I or any one or more of the foregoing embodiments any one of claims 1-18 wherein X is CH₂, R³ and R⁶ are both hydrogen and the sum of o and p is 2.
10) A tricyclic fused pyrimidine compound of Formula I or any one or more of the foregoing embodiments wherein X is CH₂, R³ and R⁶ are both hydrogen and the sum of o and p is 3.
11) A tricyclic fused pyrimidine compound of Formula I or any one or more of the foregoing embodiments wherein R⁴ is hydrogen and each R⁵ of CR⁵ is independently selected from hydrogen, methyl or methoxy, and R⁵ of NR⁵ is hydrogen or methyl.
12) A tricyclic fused pyrimidine compound of Formula I or any one or more of the foregoing embodiments wherein Y is carboxylic acid, carboxylic ester, carboxamido, sulfonoxy, sulfonamido, tetrazolyl, boronic acid or boronic ester, wherein the ester group of carboxylic ester or boronic ester is methyl or ethyl.
13) A tricyclic fused pyrimidine compound of Formula I or any one or more of the foregoing embodiments wherein D is C-Me, C—OMe, C-Et or C—OEt.
14) A tricyclic fused pyrimidine compound of Formula I or any one or more of the foregoing embodiments wherein Formula II applies.
15) A tricyclic fused pyrimidine compound of Formula I or any one or more of the foregoing embodiments wherein Formula III applies.
16) A tricyclic fused pyrimidine compound of Formula I or any one or more of the foregoing embodiments wherein formula IV applies.
17) A tricyclic fused pyrimidine compound of Formula I or any one or more of the foregoing embodiments wherein X is O.
18) A tricyclic fused pyrimidine compound of Formula I or any one or more of the foregoing embodiments wherein X is NR¹.
19) A tricyclic fused pyrimidine compound of Formula I or any one or more of the foregoing embodiments wherein X is CH₂.
20) A tricyclic fused pyrimidine compound of Formula I or any one or more of the foregoing embodiments wherein the sum of o and p is 3 so that the op ring is a 6 member ring.
21) A tricyclic fused pyrimidine compound of Formula I or any one or more of the foregoing embodiments wherein the sum of o and p is 2 so that the op ring is a 5 member ring.
22) A tricyclic fused pyrimidine compound of Formula I or any one or more of the foregoing embodiments wherein each R³ is H and R⁴ is H.
23) A tricyclic fused pyrimidine compound of Formula I or any one or more of the foregoing embodiments wherein Ar is phenyl, thiophenyl, pyridinyl, oxazole, furanyl or a mono-substituted version thereof wherein the substituent is selected from halogen or C₁ to C₄ straight or branched alkyl.
24) A tricyclic fused pyrimidine compound of Formula I or any one or more of the foregoing embodiments wherein the Ar is phenyl or fluorophenyl.
   Alternatives 25-60 specify designations for each of the variable symbols of Formula I and in some instances specify several choices for an individual symbol. Alternatives 61-68 apply to all of the preceeding subgeneric embodiments 1-60 and alternatives 69-71 apply to selected groups of the cores 1-33.
25) A tricyclic fused pyrimidine compound of Formula I or Formula II wherein o is 1; p is 2; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
26) A tricyclic fused pyrimidine compound of Formula I or Formula III wherein o is 1; p is 2; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
27) A tricyclic fused pyrimidine compound of Formula I or Formula IV wherein o is 1; p is 2; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, or —B(OME)₂; Ar is phenyl or fluorophenyl.
28) A tricyclic fused pyrimidine compound of Formula I or Formula II wherein o is 2; p is 1; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
29) A tricyclic fused pyrimidine compound of Formula I or Formula III wherein o is 2; p is 1; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
30) A tricyclic fused pyrimidine compound of Formula I or Formula IV wherein o is 2; p is 1; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
31) A tricyclic fused pyrimidine compound of Formula I or Formula II wherein o is 1; p is 2; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
32) A tricyclic fused pyrimidine compound of Formula I or Formula III wherein o is 1; p is 2; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
33) A tricyclic fused pyrimidine compound of Formula I or Formula IV wherein o is 1; p is 2; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
34) A tricyclic fused pyrimidine compound of Formula I or Formula II wherein o is 2; p is 1; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
35) A tricyclic fused pyrimidine compound of Formula I or Formula III wherein o is 2; p is 1; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
36) A tricyclic fused pyrimidine compound of Formula I or Formula IV wherein o is 2; p is 1; Z¹ is CR²; R², R³, R⁴ and R⁶ are all H; Z² is N; X is O or NH or NMe or CH₂; A is CH; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
37) A tricyclic fused pyrimidine compound of Formula I or Formula II wherein o is 1; p is 2; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
38) A tricyclic fused pyrimidine compound of Formula I or Formula III wherein o is 1; p is 2; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
39) A tricyclic fused pyrimidine compound of Formula I or Formula IV wherein o is 1; p is 2; Z¹ and Z² are both CR²; R², R³, R⁴ and R⁶ are all H; X is O or NH or NMe or CH₂; A is CH; D is CMe or COMe; E is N; Y is —COOH, —CONH₂, —SO₂NH₂, —CN, -tetrazolyl, —B(OH)₂ or —B(OME)₂; Ar is phenyl or fluorophenyl.
40) A tricyclic fused pyrimidine compound of Formula I or Formula II wherein o is 2; p is 1; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
41) A tricyclic fused pyrimidine compound of Formula I or Formula III wherein o is 2; p is 1; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
42) A tricyclic fused pyrimidine compound of Formula I or Formula IV wherein o is 2; p is 1; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
43) A tricyclic fused pyrimidine compound of Formula I or Formula II wherein o is 1; p is 2; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
44) A tricyclic fused pyrimidine compound of Formula I or Formula III wherein o is 1; p is 2; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
45) A tricyclic fused pyrimidine compound of Formula I or Formula IV wherein o is 1; p is 2; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
46) A tricyclic fused pyrimidine compound of Formula I or Formula II wherein o is 2; p is 1; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
47) A tricyclic fused pyrimidine compound of Formula I or Formula III wherein o is 2; p is 1; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
48) A tricyclic fused pyrimidine compound of Formula I or Formula IV wherein o is 2; p is 1; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
49) A tricyclic fused pyrimidine compound of Formula I or Formula II wherein o is 1; p is 2; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
50) A tricyclic fused pyrimidine compound of Formula I or Formula III wherein o is 1; p is 2; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; H, Ar is phenyl or fluorophenyl.
51) A tricyclic fused pyrimidine compound of Formula I or Formula IV wherein o is 1; p is 2; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
52) A tricyclic fused pyrimidine compound of Formula I or Formula II wherein o is 2; p is 1; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
53) A tricyclic fused pyrimidine compound of Formula I or Formula III wherein o is 2; p is 1; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
54) A tricyclic fused pyrimidine compound of Formula I or Formula IV wherein o is 2; p is 1; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
55) A tricyclic fused pyrimidine compound of Formula I or Formula II wherein o is 1; p is 2; Z¹ is CR²; R², R³, R⁴ and R⁶ are all H; Z² is N; R³ is H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
56) A tricyclic fused pyrimidine compound of Formula I or Formula III wherein o is 1; p is 2; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
57) A tricyclic fused pyrimidine compound of Formula I or Formula IV wherein o is 1; p is 2; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
58) A tricyclic fused pyrimidine compound of Formula I or Formula II wherein o is 2; p is 1; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
59) A tricyclic fused pyrimidine compound of Formula I or Formula III wherein o is 2; p is 1; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
60) A tricyclic fused pyrimidine compound of Formula I or Formula IV wherein o is 2; p is 1; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
61) A tricyclic fused pyrimidine compound of Formula I or any one or more of the foregoing subgeneric embodiments wherein Y is —CONH₂, —SO₂NH₂, -tetrazolyl or boronic acid or boronic ester.
62) A tricyclic fused pyrimidine compound of Formula I or any one or more of the foregoing subgeneric embodiments wherein D is CMe.
63) A tricyclic fused pyrimidine compound of Formula I or any one or more of the foregoing subgeneric embodiments wherein D is COMe.
64) A tricyclic fused pyrimidine compound of Formula I or any one or more of the foregoing subgeneric embodiments wherein X is O.
65) A tricyclic fused pyrimidine compound of Formula I or any one or more of the foregoing subgeneric embodiments wherein X is NH.
66) A tricyclic fused pyrimidine compound of Formula I or any one or more of the foregoing subgeneric embodiments wherein X is CH₂.
67) A tricyclic fused pyrimidine compound of Formula I or any one or more of the foregoing subgeneric embodiments Formula I or any one or more of the foregoing subgeneric embodiments wherein Z¹ and Z² are both CR² and R² is H.
68) A tricyclic fused pyrimidine compound of Formula I or any one or more of the foregoing subgeneric embodiments wherein X is CH₂.
69) A tricyclic fused pyrimidine compound of Formula I having core 19, 20, 21, 25, 26, 27, 28, 29, 30, 31, each core having the P2 and P4 groups and the R groups as indicated for Formula I or any subgeneric or preferred version thereof.
70) A tricyclic fused pyrimidine compound of Formula I of statement 69 wherein the core is core 19, 20, 21 or 31.
71) A tricyclic fused pyrimidine compound of Formula I having core 22, 23, 24, or 32, each core having the P2 and P4 groups and the R groups as indicated for Formula I or any subgeneric or preferred version thereof.

### Preferred Individual Tricyclic Compounds

1-(4-(benzylamino)-6,8,9,10,11,11a-hexahydro-5H-pyrimido[4,5-a]quinolizin-2-yl)-2-methyl-1H-indole-4-carboxamide
1-(4-((3-fluorobenzyl)amino)-6,8,9,10,11,11a-hexahydro-5H-pyrimido[4,5-a]quinolizin-2-yl)-2-methyl-1H-indole-4-carboxamide
(1-(4-(benzylamino)-6,8,9,10,11,11a-hexahydro-5H-pyrimido[4,5-a]quinolizin-2-yl)-2-methyl-1H-indol-4-yl)boronic acid
(1-(4-((3-fluorobenzyl)amino)-6,8,9,10,11,11a-hexahydro-5H-pyrimido[4,5-a]quinolizin-2-yl)-2-methyl-1H-indol-4-yl)boronic acid
1-(4-(benzylamino)-6,6a,7,8,9,10-hexahydro-5H-pyrimido[5,4-c]quinolizin-2-yl)-2-methyl-1H-indole-4-carboxamide
1-(4-((3-fluorobenzyl)amino)-6,6a,7,8,9,10-hexahydro-5H-pyrimido[5,4-c]quinolizin-2-yl)-2-methyl-1H-indole-4-carboxamide
(1-(4-(benzylamino)-6,6a,7,8,9,10-hexahydro-5H-pyrimido[5,4-c]quinolizin-2-yl)-2-methyl-1H-indol-4-yl)boronic acid
(1-(4-((3-fluorobenzyl)amino)-6,6a,7,8,9,10-hexahydro-5H-pyrimido[5,4-c]quinolizin-2-yl)-2-methyl-1H-indol-4-yl)boronic acid
1-(4-(benzylamino)-5a,6,7,8,9,11-hexahydro-5H-pyrimido[5,4-b]quinolizin-2-yl)-2-methyl-1H-indole-4-carboxamide
1-(4-((3-fluorobenzyl)amino)-5a,6,7,8,9,11-hexahydro-5H-pyrimido[5,4-b]quinolizin-2-yl)-2-methyl-1H-indole-4-carboxamide
(1-(4-(benzylamino)-5a,6,7,8,9,11-hexahydro-5H-pyrimido[5,4-b]quinolizin-2-yl)-2-methyl-1H-indol-4-yl)boronic acid
(1-(4-((3-fluorobenzyl)amino)-5a,6,7,8,9,11-hexahydro-5H-pyrimido[5,4-b]quinolizin-2-yl)-2-methyl-1H-indol-4-yl)boronic acid
1-(4-(benzylamino)-5,5a,6,8,9,11-hexahydropyrimido[5′,4′:4,5]pyrido[2,1-c][1,4]oxazin-2-yl)-2-methyl-1H-indole-4-carboxamide
1-(4-(benzylamino)-5a,6,7,8,9,11-hexahydro-5H-pyrazino[1′,2′:1,6]pyrido[3,4-d]pyrimidin-2-yl)-2-methyl-1H-indole-4-carboxamide
1-(4-(benzylamino)-7,8,9,10,10a,11-hexahydro-5H-pyrimido[4,5-b]quinolizin-2-yl)-2-methyl-1H-indole-4-carboxamide
1-(4-((3-fluorobenzyl)amino)-7,8,9,10,10a,11-hexahydro-5H-pyrimido[4,5-b]quinolizin-2-yl)-2-methyl-1H-indole-4-carboxamide
(1-(4-(benzylamino)-7,8,9,10,10a,11-hexahydro-5H-pyrimido[4,5-b]quinolizin-2-yl)-2-methyl-1H-indol-4-yl)boronic acid
(1-(4-((3-fluorobenzyl)amino)-7,8,9,10,10a,11-hexahydro-5H-pyrimido[4,5-b]quinolizin-2-yl)-2-methyl-1H-indol-4-yl)boronic acid
1-(4-(benzylamino)-5,7,8,10,10a,11-hexahydropyrimido[4′,5′:4,5]pyrido[2,1-c][1,4]oxazin-2-yl)-2-methyl-1H-indole-4-carboxamide
1-(4-(benzylamino)-7,8,9,10,10a,11-hexahydro-5H-pyrazino[1′,2′:1,6]pyrido[4,3-d]pyrimidin-2-yl)-2-methyl-1H-indole-4-carboxamide
1-(1-(benzylamino)-6,8,9,10,11,11a-hexahydro-5H-pyrimido[5,4-a]quinolizin-3-yl)-2-methyl-1H-indole-4-carboxamide
1-(1-((3-fluorobenzyl)amino)-6,8,9,10,11,11a-hexahydro-5H-pyrimido[5,4-a]quinolizin-3-yl)-2-methyl-1H-indole-4-carboxamide
1-(1-(benzylamino)-5,6,8,9,11,11a-hexahydropyrimido[5′,4′:3,4]pyrido[2,1-c][1,4]oxazin-3-yl)-2-methyl-1H-indole-4-carboxamide
1-(1-(benzylamino)-6,8,9,10,11,11a-hexahydro-5H-pyrazino[1′,2′:1,2]pyrido[4,3-d]pyrimidin-3-yl)-2-methyl-1H-indole-4-carboxamide
(1-(1-(benzylamino)-6,8,9,10,11,11a-hexahydro-5H-pyrimido[5,4-a]quinolizin-3-yl)-2-methyl-1H-indol-4-yl)boronic acid
(1-(1-((3-fluorobenzyl)amino)-6,8,9,10,11,11a-hexahydro-5H-pyrimido[5,4-a]quinolizin-3-yl)-2-methyl-1H-indol-4-yl)boronic acid
1-(1-(benzylamino)-6,6a,7,8,9,10-hexahydro-5H-pyrimido[4,5-c]quinolizin-3-yl)-2-methyl-1H-indole-4-carboxamide
1-(1-((3-fluorobenzyl)amino)-6,6a,7,8,9,10-hexahydro-5H-pyrimido[4,5-c]quinolizin-3-yl)-2-methyl-1H-indole-4-carboxamide
(1-(1-(benzylamino)-6,6a,7,8,9,10-hexahydro-5H-pyrimido[4,5-c]quinolizin-3-yl)-2-methyl-1H-indol-4-yl)boronic acid
(1-(1-((3-fluorobenzyl)amino)-6,6a,7,8,9,10-hexahydro-5H-pyrimido[4,5-c]quinolizin-3-yl)-2-methyl-1H-indol-4-yl)boronic acid
1-(1-((3-fluorobenzyl)amino)-5,6,8,9,10,10a-hexahydropyrimido[4,5-g]indolizin-3-yl)-2-methyl-1H-indole-4-carboxamide
1-(1-(benzylamino)-5,6,8,9,10,10a-hexahydropyrimido[4,5-g]indolizin-3-yl)-2-methyl-1H-indole-4-carboxamide
(1-(1-(benzylamino)-5,6,8,9,10,10a-hexahydropyrimido[4,5-g]indolizin-3-yl)-2-methyl-1H-indol-4-yl)boronic acid
(1-(1-((3-fluorobenzyl)amino)-5,6,8,9,10,10a-hexahydropyrimido[4,5-g]indolizin-3-yl)-2-methyl-1H-indol-4-yl)boronic acid
1-(4-(benzylamino)-5,7,8,9,9a,10-hexahydropyrimido[5,4-f]indolizin-2-yl)-2-methyl-1H-indole-4-carboxamide
1-(4-((3-fluorobenzyl)amino)-5,7,8,9,9a,10-hexahydropyrimido[5,4-f]indolizin-2-yl)-2-methyl-1H-indole-4-carboxamide
(1-(4-(benzylamino)-5,7,8,9,9a,10-hexahydropyrimido[5,4-f]indolizin-2-yl)-2-methyl-1H-indol-4-yl)boronic acid
(1-(4-((3-fluorobenzyl)amino)-5,7,8,9,9a,10-hexahydropyrimido[5,4-f]indolizin-2-yl)-2-methyl-1H-indol-4-yl)boronic acid
1-(4-(benzylamino)-5,5a,6,7,8,10-hexahydropyrimido[4,5-f]indolizin-2-yl)-2-methyl-1H-indole-4-carboxamide
1-(4-((3-fluorobenzyl)amino)-5,5a,6,7,8,10-hexahydropyrimido[4,5-f]indolizin-2-yl)-2-methyl-1H-indole-4-carboxamide
(1-(4-(benzylamino)-5,5a,6,7,8,10-hexahydropyrimido[4,5-f]indolizin-2-yl)-2-methyl-1H-indol-4-yl)boronic acid
(1-(4-((3-fluorobenzyl)amino)-5,5a,6,7,8,10-hexahydropyrimido[4,5-f]indolizin-2-yl)-2-methyl-1H-indol-4-yl)boronic acid
1-(4-(benzylamino)-5,6,8,9,10,10a-hexahydropyrimido[5,4-g]indolizin-2-yl)-2-methyl-1H-indole-4-carboxamide
1-(4-((3-fluorobenzyl)amino)-5,6,8,9,10,10a-hexahydropyrimido[5,4-g]indolizin-2-yl)-2-methyl-1H-indole-4-carboxamide
(1-(4-(benzylamino)-5,6,8,9,10,10a-hexahydropyrimido[5,4-g]indolizin-2-yl)-2-methyl-1H-indol-4-yl)boronic acid
(1-(4-((3-fluorobenzyl)amino)-5,6,8,9,10,10a-hexahydropyrimido[5,4-g]indolizin-2-yl)-2-methyl-1H-indol-4-yl)boronic acid
1-(4-(benzylamino)-5,6,6a,7,8,9-hexahydropyrimido[4,5-e]indolizin-2-yl)-2-methyl-1H-indole-4-carboxamide
1-(4-((3-fluorobenzyl)amino)-5,6,6a,7,8,9-hexahydropyrimido[4,5-e]indolizin-2-yl)-2-methyl-1H-indole-4-carboxamide
(1-(4-(benzylamino)-5,6,6a,7,8,9-hexahydropyrimido[4,5-e]indolizin-2-yl)-2-methyl-1H-indol-4-yl)boronic acid
(1-(4-((3-fluorobenzyl)amino)-5,6,6a7,8,9-hexahydropyrimido[4,5-e]indolizin-2-yl)-2-methyl-1H-indol-4-yl)boronic acid
1-(4-(benzylamino)-5,5a,6,7,8,9,9a,10-octahydrobenzo[g]quinazolin-2-yl)-2-methyl-1H-indole-4-carboxamide
(1-(4-(benzylamino)-5,5a,6,7,8,9,9a,10-octahydrobenzo[g]quinazolin-2-yl)-2-methyl-1H-indo-4-yl)boronic acid
1-(4-(benzylamino)-5a,6,8,9,9a,10-hexahydro-5H-pyrano[3,4-g]quinazolin-2-yl)-2-methyl-1H-indole-4-carboxamide
1-(4-(benzylamino)-5,5a,6,7,8,9,9a,10-octahydropyrido[3,4-g]quinazolin-2-yl)-2-methyl-1H-indole-4-carboxamide
(1-(4-(benzylamino)-5a,6,8,9,9a,10-hexahydro-5H-pyrano[3,4-g]quinazolin-2-yl)-2-methyl-1H-indol-4-yl)boronic acid
(1-(4-(benzylamino)-5,5a,6,7,8,9,9a,10-octahydropyrido[3,4-g]quinazolin-2-yl)-2-methyl-1H-indol-1-yl)boronic acid
1-(4-(benzylamino)-5a,6,7,9,9a,10-hexahydro-5H-pyrano[4,3-g]quinazolin-2-yl)-2-methyl-1H-indole-4-carboxamide
1-(4-(benzylamino)-5,5a,6,7,8,9,9a,10-octahydropyrido[4,3-g]quinazolin-2-yl)-2-methyl-1H-indole-4-carboxamide
(1-(4-(benzylamino)-5a,6,7,9,9a,10-hexahydro-5H-pyrano[4,3-g]quinazolin-2-yl)-2-methyl-1H-indol-4-yl)boronic acid
(1-(4-(benzylamino)-5,5a,6,7,8,9,9a,10-octahydropyrido[4,3-g]quinazolin-2-yl)-2-methyl-1H-indol-1-yl)boronic acid
1-(4-(benzylamino)-4b,5,6,7,8,10-hexahydropyrimido[5,4-a]indolizin-2-yl)-2-methyl-1H-indole-4-carboxamide
1-(4-(benzylamino)-6,7,8,9,9a,10-hexahydropyrimido[4,5-b]indolizin-2-yl)-2-methyl-1H-indole-4-carboxamide
(1-(4-(benzylamino)-6,7,8,9,9a,10-hexahydropyrimido[4,5-b]indolizin-2-yl)-2-methyl-1H-indol-4-yl)boronic acid
(1-(4-(benzylamino)-4b,5,6,7,8,10-hexahydropyrimido[5,4-a]indolizin-2-yl)-2-methyl-1H-indol-4-yl)boronic acid
1-(4-(benzylamino)-5,7,8,9,10,10a-hexahydropyrimido[4,5-a]indolizin-2-yl)-2-methyl-1H-indole-4-carboxamide
1-(4-(benzylamino)-5,5a,6,7,8,9-hexahydropyrimido[5,4-b]indolizin-2-yl)-2-methyl-1H-indole-4-carboxamide
(1-(4-(benzylamino)-5,5a,6,7,8,9-hexahydropyrimido[5,4-b]indolizin-2-yl)-2-methyl-1H-indol-4-yl)boronic acid
(1-(4-(benzylamino)-5,5a,6,7,8,9-hexahydropyrimido[5,4-b]indolizin-2-yl)-2-methyl-1H-indol-4-yl)boronic acid

### General Synthetic Schemes for Mono and Bicyclic Compounds

Compounds of the present invention can be synthesized using the following methods. General reaction conditions are given and reaction products can be purified by general known methods including crystallization, silica gel chromatography using various organic solvents such as hexane, cyclohexane, ethyl acetate, methanol and the like, preparative high pressure liquid chromatography or preparative reverse phase high pressure liquid chromatography.

The synthesis of target molecules is illustrated was achieved through a variety of palladium-catalyzed coupling reactions between 2-chloro-4-benzylamino (substituted) fused tricyclic fused pyrimidines or their derivatives (herein referred as the cores) with the 5,6-bicycloaromatic rings (herein referred as P2-moieties) to introduce this functionality at the P-2 position.

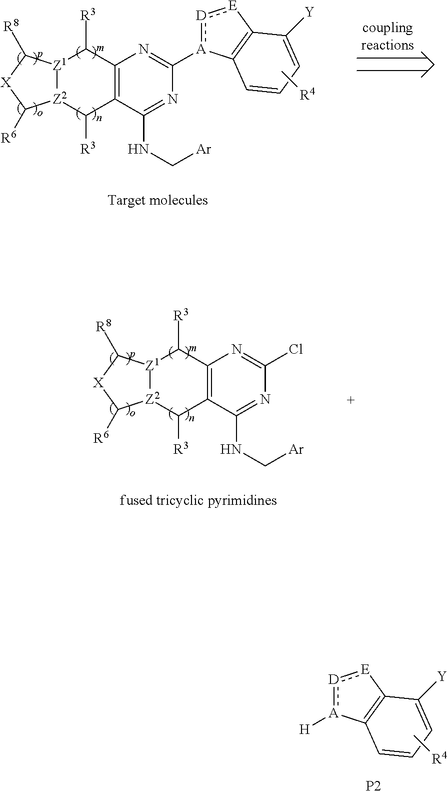

Representative synthetic routes of the (substituted) fused tricyclic fused pyrimidines are illustrated as the following.

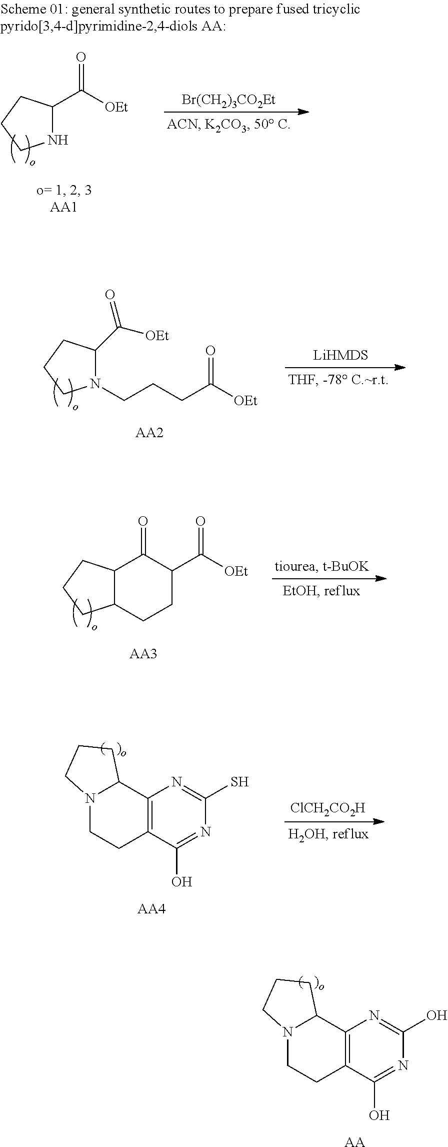

A 2-carboxylate of the general structure AA1 reacted with ethyl 4-bromobutanoate to install the moiety onto the nitrogen and form intermediate AA2. Under −78° C., treatment of the latter with a strong base such as LiHMDS resulted in intramolecular cyclization to yield the ketoester AA3 in reasonable yield. Then AA3 reacted with thiourea in the presence of t-BuOK to yield 2-mercaptopyrimidin-4-ol, which was easily converted into the diolAA.

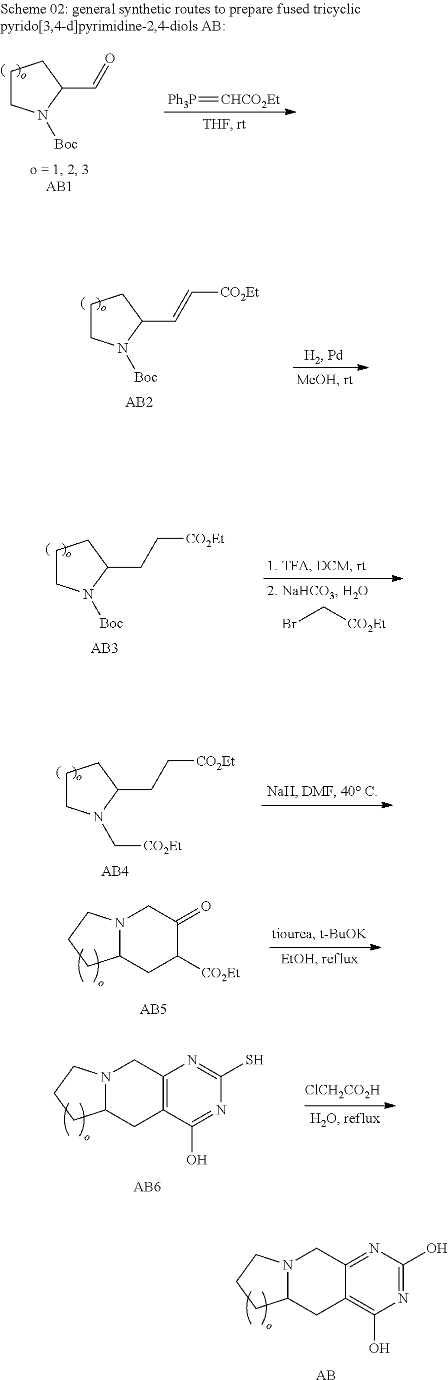

A 2-aldehyde of the general structure AB1 reacted with (carbethoxymethylene) triphenyl-phosphorane followed by reduction by hydrogen in the presence of Pd to yield AB3 using methods similar to those described in Larry Wendell Hardy et al, PCT Int. Appl., 2011075699. Then its Boc protection group was removed and reacted with ethyl 2-bromoacetate to yield diesters AB4, and the latter was treated with sodium hydride to yield ketoester AB5. A similar procedure as aforementioned was implemented to covert the ketoester into the diol AB.

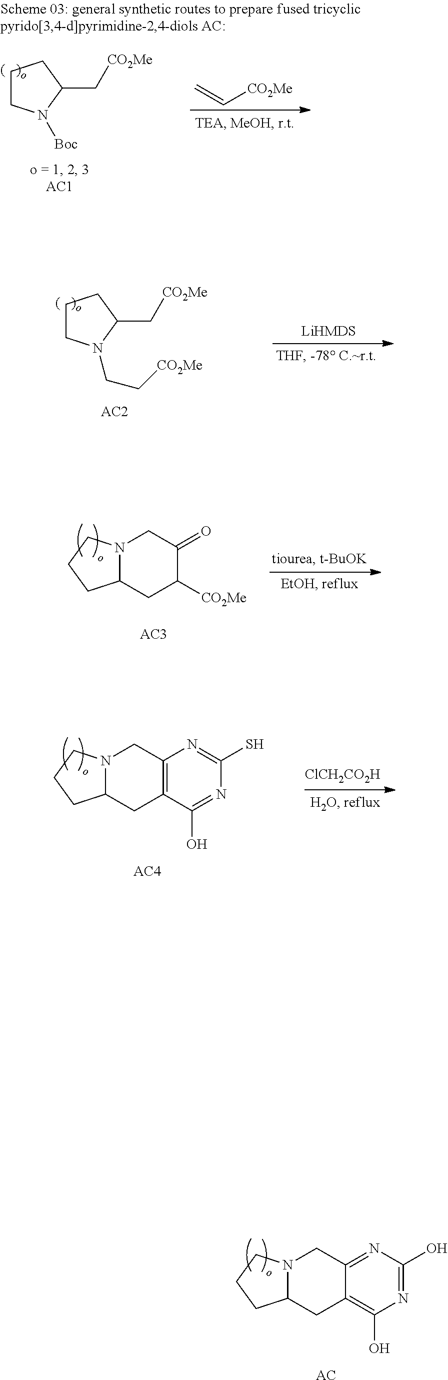

A 2-acetate of the general structure AC1 reacted with ethyl acrylate and triethylamine at room temperature to install the moiety of propanoate onto the nitrogen AC2 using methods similar to those described in Milan Chytil et al PCT Int. Appl., 2010144571. Under −78° C., treatment of the latter with a strong base such as LiHMDS resulted in intramolecular cyclization to yield the ketoester AC3. A similar procedure as aforementioned was implemented to covert the ketoester into the diol AC.

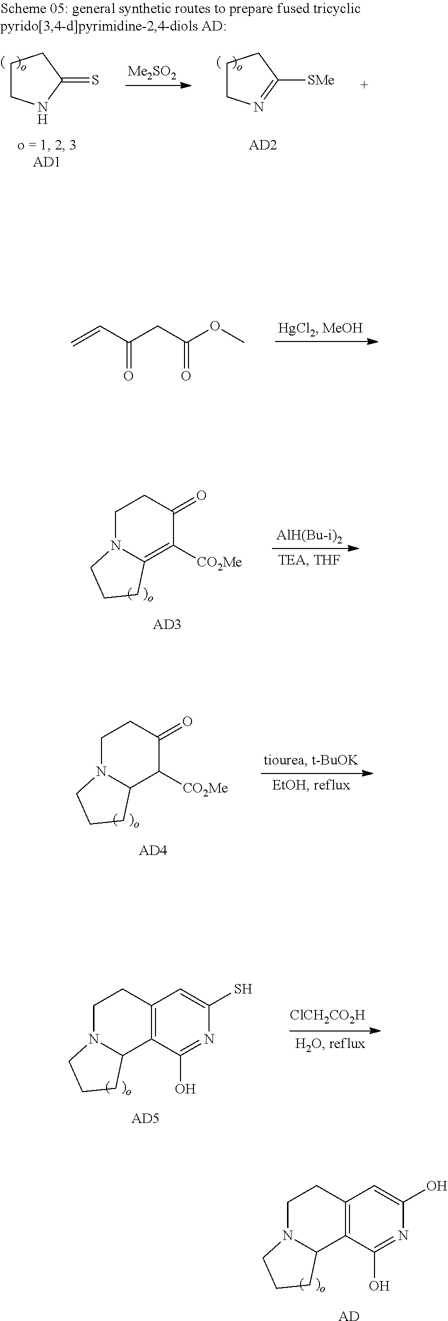

A thiolactam of the general structure AD1 was transformed into methylthioimine AD2. Then the latter was converted into ketoester AD4 via a two-step procedure similar to those described in Hiroki Takahata et al, Chemical & Pharmaceutical Bulletin, 1986, 34, 4523. A similar procedure as aforementioned was implemented to covert the ketoester into the diol AD.

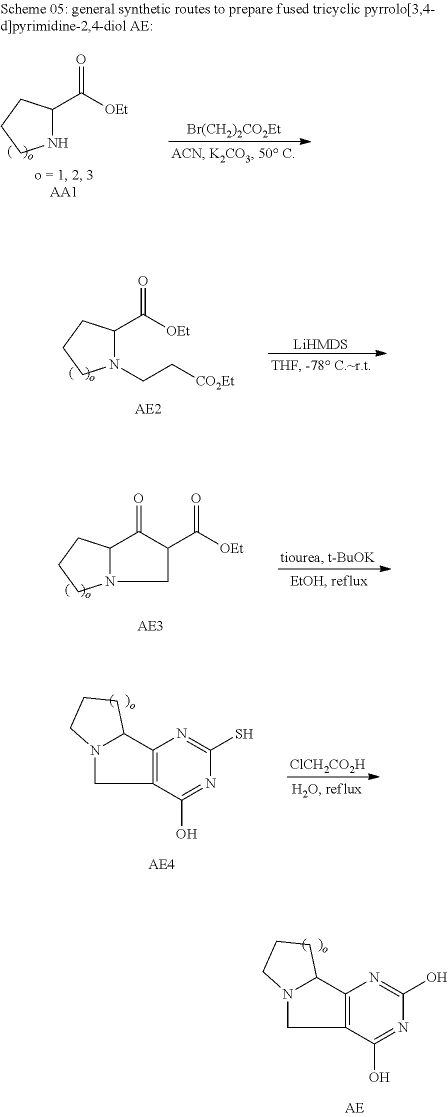

A 2-carboxylate of the general structure AA1 reacted with 3-bromopropanoate to install the moiety onto the nitrogen and form intermediates AE2. Under −78° C., treatment of the latter with a strong base such as LiHMDS resulted in intramolecular cyclization to yield the ketoester AE3 in reasonable yield. Then AE3 reacted with thiourea in the presence of t-BuOK to yield 2-mercaptopyrimidin-4-ol AE4, which was easily converted into the diol AE.

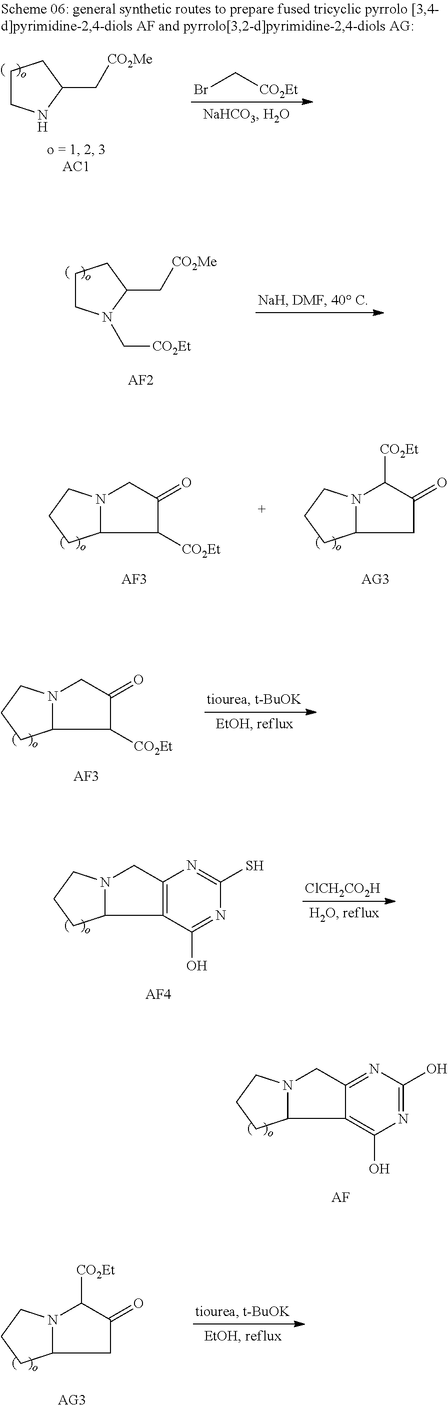

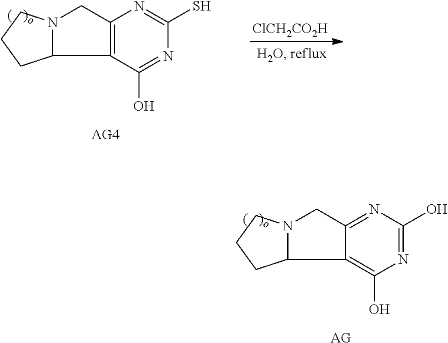

A 2-acetate of the general structure AC1 reacted with 2-bromoacetate to install the moiety onto the nitrogen and form intermediates AF2. And the latter was treated with strong bases such as sodium hydride to yield a mixture of ketoester AF3 and AG3. Then they were converted into diols AF and AG respectively via a similar procedure as aforementioned.

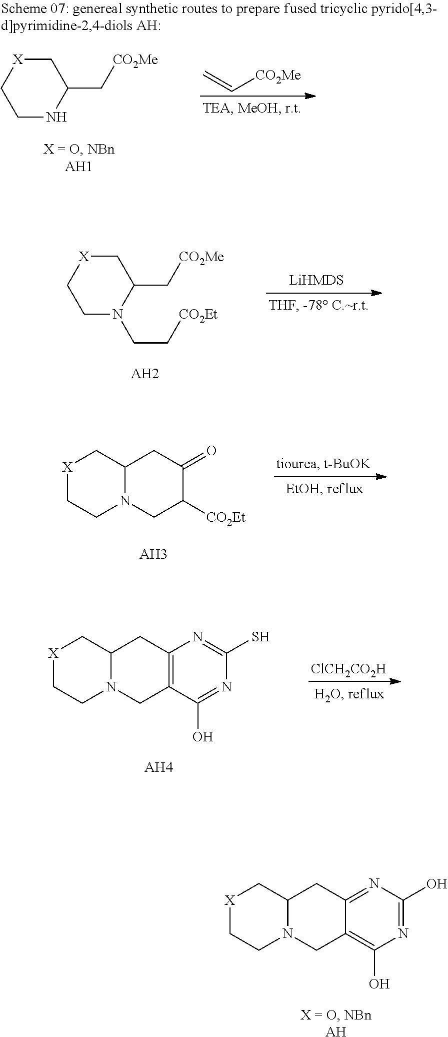

A 2-acetate of the general structure AH1 (morpholin-3-yl or Bn-protected piperazin-2-yl, X=O or NBn) reacted with ethyl acrylate and triethylamine at room temperature to install the moiety of propanoate onto the nitrogen AC2 using methods similar to those described in Ralf Anderskewitz et al PCT Int. Appl., 2014140075. Under −78° C., treatment of the latter with a strong base such as LiHMDS resulted in intramolecular cyclization to yield the ketoester AH3. A similar procedure as aforementioned was implemented to covert the ketoester into the diol AH.

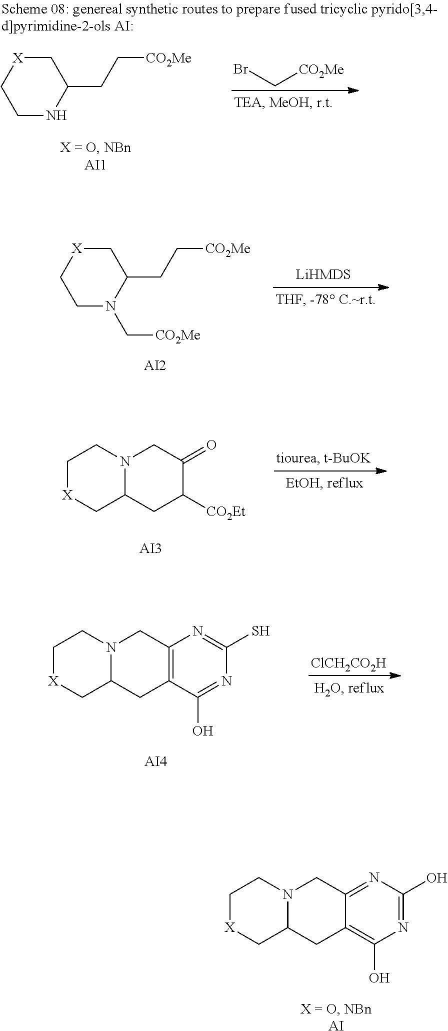

A 2-propanate of the general structure AI1 (morpholin-3-yl or Bn-protected piperazin-2-yl, X=O or NBn) reacted with 2-bromoacetate to install the moiety of acetate onto the nitrogen AI2. Under −78° C., treatment of the latter with a strong base such as LiHMDS resulted in intramolecular cyclization to yield the ketoester AI3. A similar procedure as aforementioned was implemented to covert the ketoester into the diol AI.

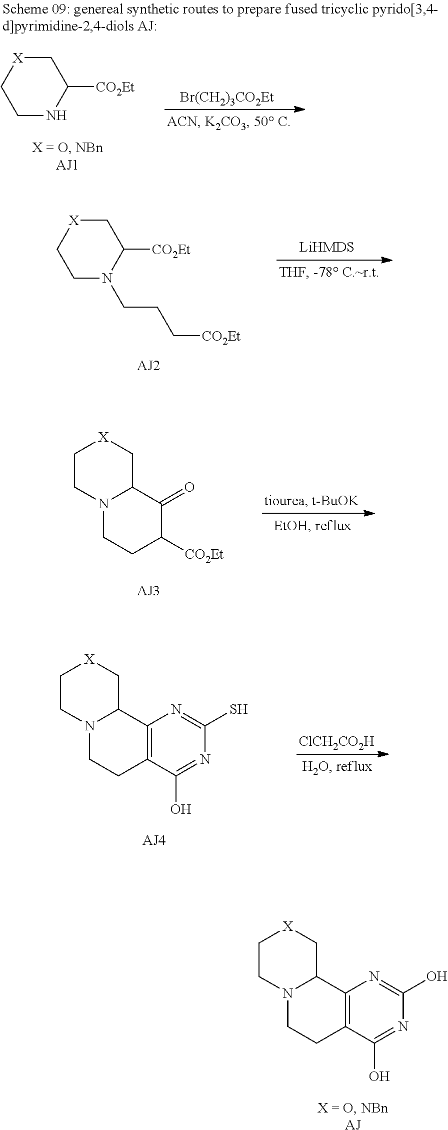

A 2-carboxylate of the general structure AJ1 (morpholin-3-yl or Bn-protected piperazin-2-yl, X=O or NBn) reacted with ethyl 4-bromobutanoate to install the moiety onto the nitrogen and form intermediates AJ2. Under −78° C., treatment of the latter with a strong base such as LiHMDS initiated intramolecular cyclization to yield the ketoester AJ3. Then AJ3 reacted with thiourea in the presence of t-BuOK to yield 2-mercaptopyrimidin-4-ol AJ4, which was easily converted into the diol AJ.

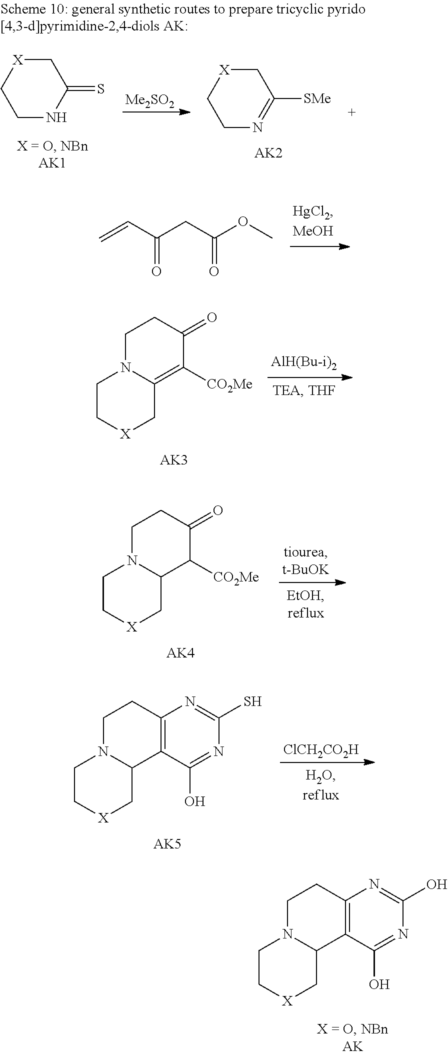

A thiolactam of the general structure AK1 (morpholin-3-yl or Bn-protected (piperazin-2-yl, X=O or NBn) was transformed into methylthioimine AK2. Then the latter was converted into ketoester AK4 via a two-step procedure similar to those described in Hiroki Takahata et al. Chemical & Pharmaceutical Bulletin, 1986, 34, 4523. A similar procedure as aforementioned was implemented to covert the ketoester into the diol AK.

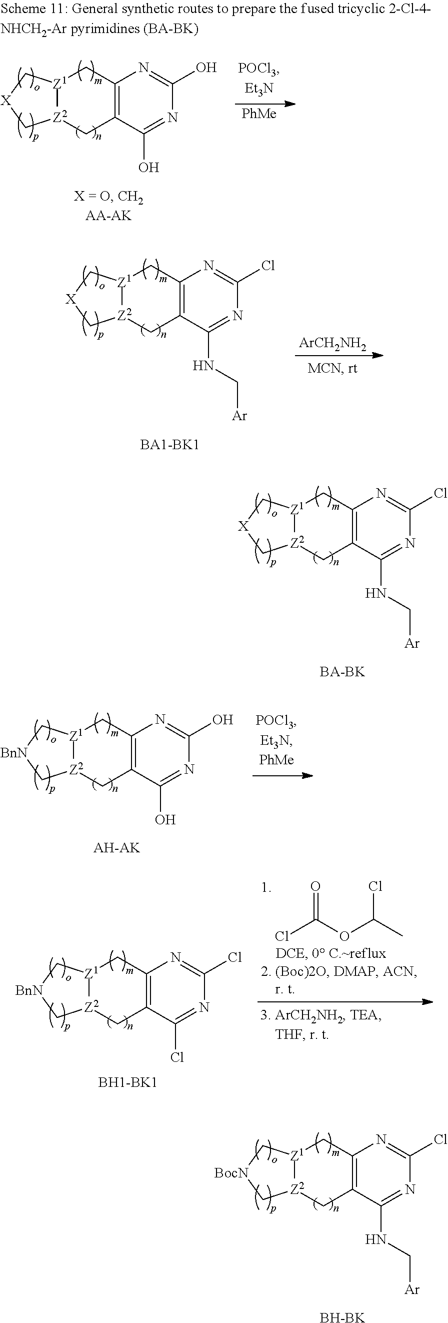

If X is oxygen or sp³ carbon, intermediates AA-AK can be converted to the dichloropyrimidine derivatives by heating them in refluxing POCl3 optionally in the presence of a tertiary amine followed by reacting with amines (NH₂CH₂Ar) to yield fused tricyclic 2-chloro-4-amino-pyrimidine of structure BA-BK (X=CH₂ or O). If X is Bn-protected amino group, Benzyl protected diols AH-AK were first transferred into the dichloride prior to converting the benzyl protecting group N-Boc group. The Boc-protected dichloride reacted with amines (NH₂CH₂Ar) to yield Boc-protected fused tricyclic 2-chloro-4-amino-pyrimidine of structure BH-BK (X=NBoc).

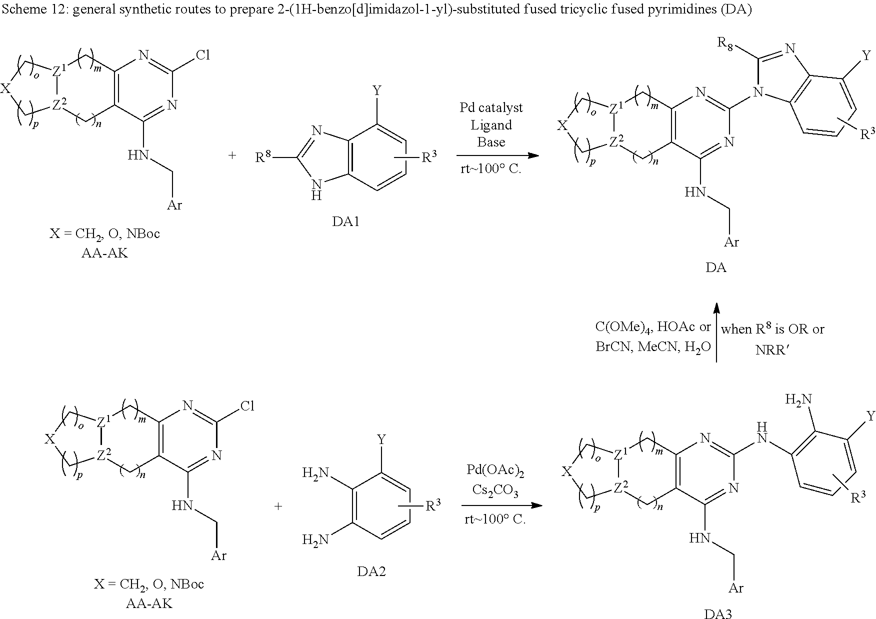

A general synthetic approach to attach benzo[d]imidazole DA1 through its 1-position into the 2-position of a fused pyrimidine to yield the desired molecules DA uses the Pd-based coupling reaction. A typical procedure employs Pd(dba)₂ as a transition metal catalyst and X-phos as a ligand and cesium carbonate as a base and dioxane an organic solvent. The reaction temperature varies from the room temperature to reflux. If X is a Boc-protected nitrogen, an extra step to deprotect was practiced. Also if Y is a nitrile (CN), it was converted to an amide in the presence of urea hydrogen peroxide (UHP). In the case where R⁸ is an alkoxy or amino group, coupling reaction can accomplished between the 2-chloro-pyrimidines AA-AK and benzene-1,2-diamines DA2 using Pd(OAc)₂ as the catalyst and Cs₂CO₃ as the base. Cyclization can occur with either bromocyanide or tetramethoxymethane.

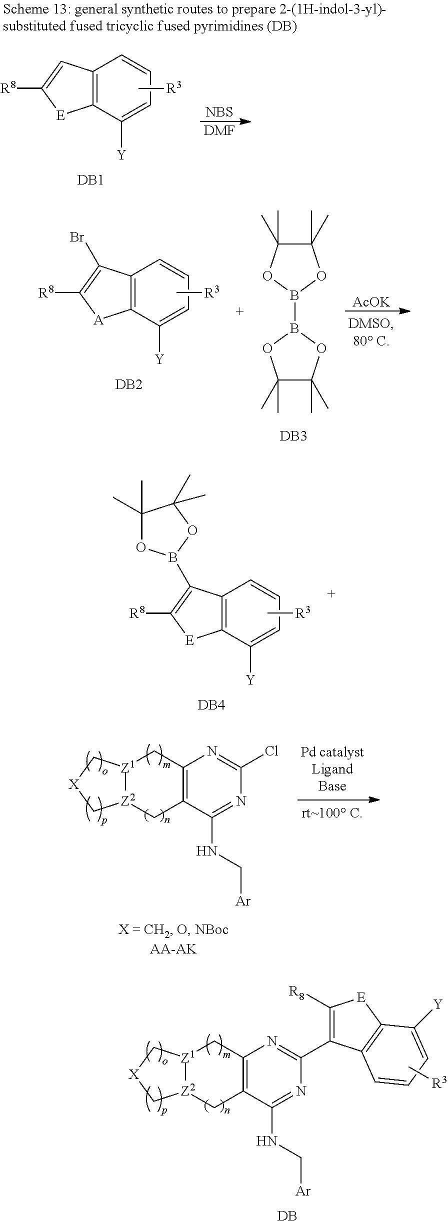

Under various conditions such as NBS in DMF, bromination of 3-unsubstituted intermediates DB1 (E as substituted or properly protected nitrogen) would take place region-selectively on the 3-position to yield 3-Br-substituted intermediates DB2. These intermediates then were converted into boronic esters DB4 by treatment with boronic ester DB3 under various conditions. Then Pd-based coupling reaction similar to those described in Zhou, H.-J. et al WO 2014015291 between intermediates DB4 and AA-AK provided the desired molecules. For example if either of E or X is protected nitrogen, an extra step for deprotection can be performed using reported conditions.

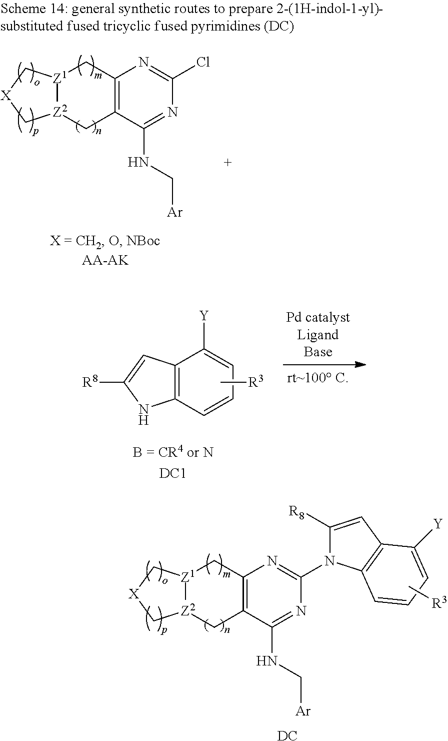

Coupling to a solution of the substituted pyrimidine AA-AK with an indole DC1 can be effective to achieve the desired molecules DC using methods similar to that described in Zhou, H.-J. et. al. WO 2014015291. For example if X is protected nitrogen, an extra step for deprotection can be performed using reported conditions.

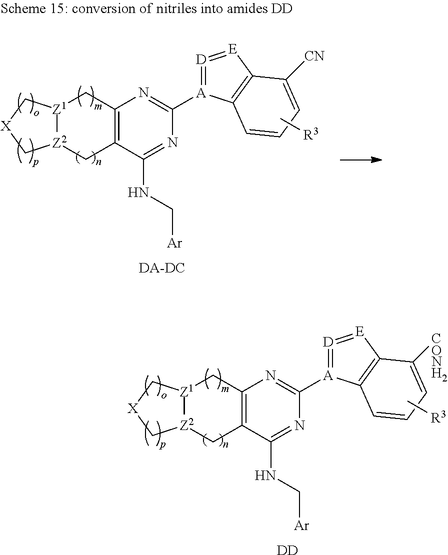

In some cases the desired compounds DA-DC prepared in Schemes 12-14 above can have a nitrile substitution at the position indicated in Scheme 15. This substituent can be converted to the corresponding carboxamide DD. Nitriles DA-DC are dissolved in a 1/10 ratio of water/DMSO and treated with urea-hydrogen peroxide (UHP) and a base such as potassium carbonate. Reaction mixture is stirred at room temperature for up to 18 hours and then is poured into ice water and stirred for two hours. The resulting solid is filtered, dried and if necessary purified by column chromatography to give the desired amides DD. For example if X is protected nitrogen, an extra step for deprotection can be performed using reported conditions.

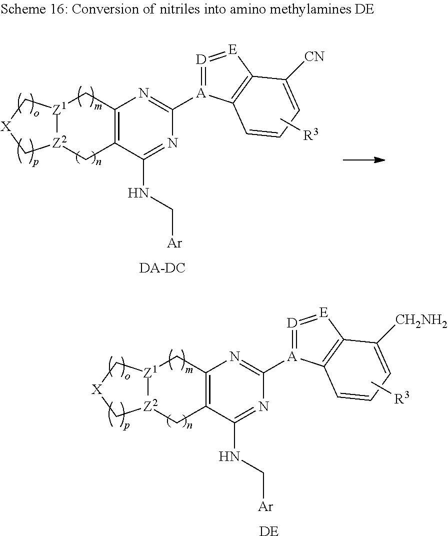

In some cases the desired compounds DA-DC prepared in Schemes 12-14 above can have a nitrile substitution at the position indicated in Scheme 16. This substituent can be converted to the corresponding methylamines DE. A solution of nitrile DA-DC in an aprotic organic solvent such as THF is treated with LAH and the resulting mixture is stirred for up to 18 hours. The reaction mixture is treated with 15% NaOH in water and the reaction is stirred for one hour and is then filtered. The THF is removed under reduced pressure to give the product DE which can be further purified by column chromatography. For example if X is protected nitrogen, an extra step for deprotection can be performed using reported conditions.

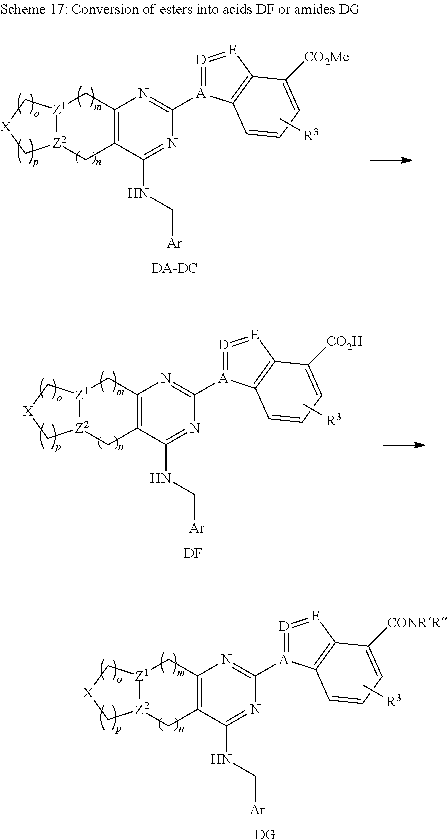

In some cases the desired compounds DA-DC prepared in Schemes 12-14 above can have a nitrile substitution at the position indicated in Scheme 17. This functionality can be readily converted to the corresponding acids DF or substituted amides DG using standard methodology. For example if X is protected nitrogen, an extra step for deprotection can be performed using reported conditions.

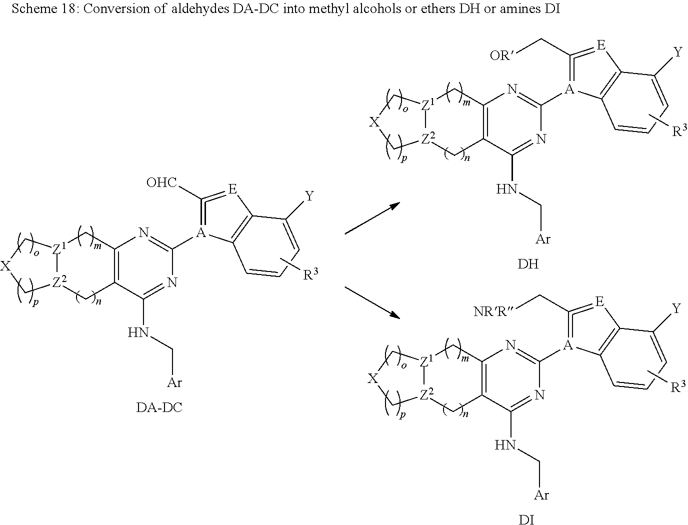

In some cases the desired compounds DA-DC prepared in Schemes 12-14 above above can have an aldehyde at the position indicated in Scheme 18. This functionality can be readily converted to the corresponding alcohols or ethers DH or amines DI or using standard methodology. For example if X is protected nitrogen, an extra step for deprotection can be performed using reported conditions.

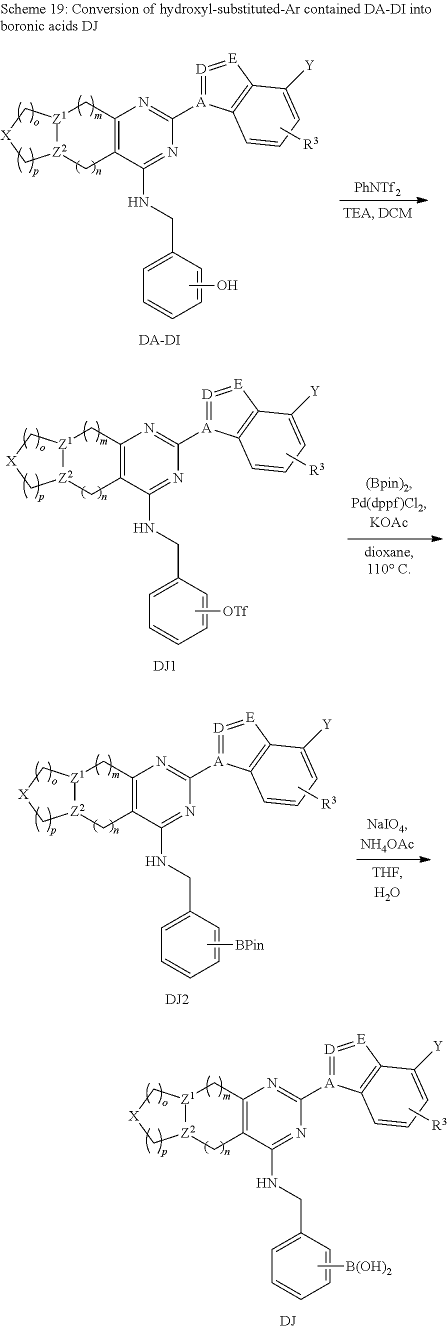

In some cases the desired compounds DA-DI prepared in Schemes 12-18 above can have a hydroxyl group substituted on Ar moiety indicated in Scheme 19. This functionality can be readily converted to the corresponding triflate DJ1, then a palladium-based coupling reaction between DJ1 and boronate ester to yield the boronate DJ2, which can be converted into boronic acid DJ.

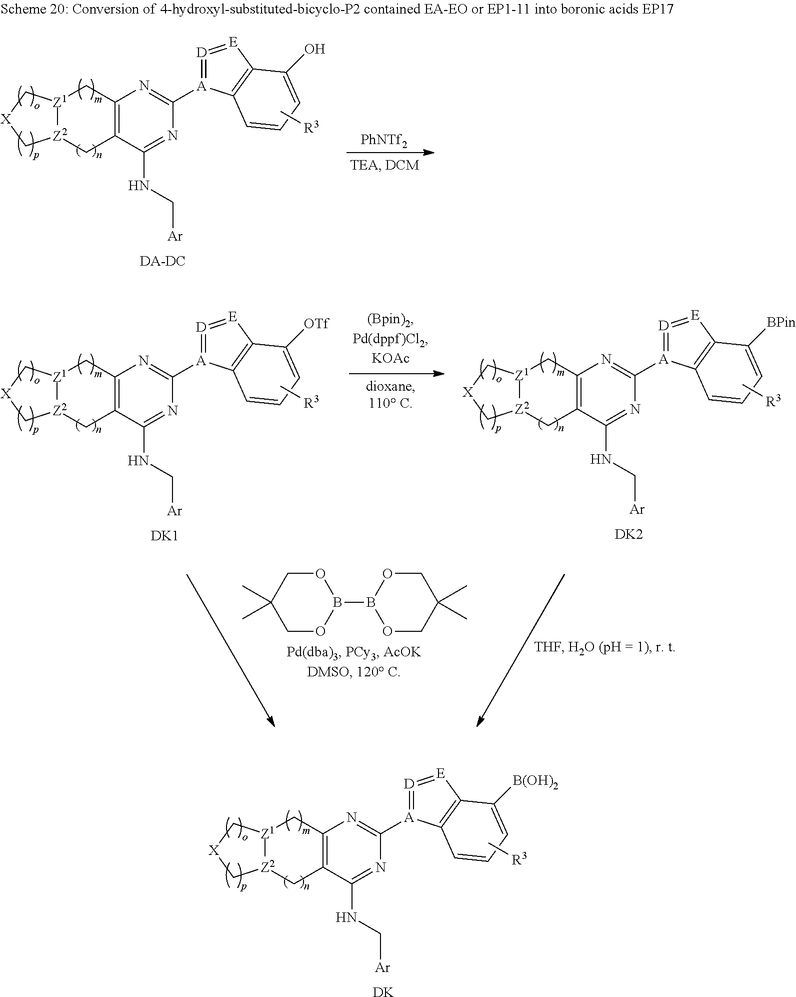

In some cases the desired compounds DA-DI prepared in Schemes 12-18 above can have a hydroxyl group substituted on Ar moiety indicated in Scheme 20. They can be converted into a boronic ester. This conversion can be performed by forming the corresponding triflate DK1 from the hydroxyl group. A palladium-based coupling reaction between DK1 and a boronate yields either boronate DK2 or boronic acid DK. Compound DK2 can be easily converted into boronic acid DK as well.

### Biological Assays

The biological activities of the tricyclic compounds of the invention can be determined by their examination in in vitro and cellular assays using protocols well established to identify and select compounds that will exhibit anti-cancer activity. The present invention focuses upon the ability of the tricyclic compounds to intersect with the p97 proteosome complex. As described in the Background, the function of the p97 complex is essential for continued cellular viability. Inhibition of the activity of the complex will cause protein build-up in the cell and consequent apoptosis. The biological assays allow an assessment of the biological activities of the tricyclic compounds of the invention.

The primary biological analyses are in vitro assays and cellular based assays for determining the inhibitory capability of the compounds of the invention of the invention against Valosin-containing protein, i.e., p97. The assays also provide a primary indication of bioavailability of the tricyclic compounds of the invention.

The ability to inhibit the p97 complex is studied through use of a p97 in vitro assay using a tagged p97 substrate pursuant to the method of Christianson in Nat Cell Biol. (2011) 14:93 for a p97 cell-based assay. A cell based assay is used to test the anti-tumor effects of inhibitors on cultured cancer cells. This anti-tumor assay is based upon cultured cancer cells using the commercially available cell titer glo assay provided by Promega. Additional assays enable assessment of bioavailability through art recognized model studies designed to demonstrate the ability of the compounds of the invention to reach target cells in vivo. While all compounds tested displayed a degree of anti-tumor activity, the assays also allowed identification of compounds of the invention as candidates that may be selected for further examined by in vivo anti-tumor testing in mouse, guinea pig and dog models. The selected candidates were shown to have highly desirable pharmacokinetic properties in these in vitro assays.

### P97 ATPase Biochemical Assay

The ATPase assay is performed according the following protocol: Purified enzyme (20 nM p97), substrate (20 μM ATP) and a dose titration of compounds are mixed in buffer (50 mM TRIS pH 7.5, 20 mM MgCl₂, 0.02% TX-100, 1 mM DTT, 0.2% (v/v) glycerol) and incubated at 37° C. for 15 minutes. The reaction is terminated and the level of product generated is measured using the ADP Glo Assay Kit (Promega, Madison Wis.). Plotting product generated versus compound concentration and using a four-parameter fit model generates an IC50 value for each compounds.

### P97 Cell-Based Assay

On target cell-based effects of compounds of the invention are monitored using the reporter cell line HEK-293 TCRα-GFP as described in Christianson et al. Nat. Cell Biol. (2011) 14:93. Inhibition of turnover of the TCRα-GFP reporter is a hallmark of p97 inhibition. The protocol for TCRα-GFP monitoring reporter turnover is as follows: Reporter cells are seeded and incubated with proteasome inhibitor MG132 to accumulate TCRα-GFP. Subsequently, MG132-containing media is removed and a dose titration of compound plus cycloheximide is incubated with the cells. At the end of the incubation, compound and media are removed, cells are fixed and GFP fluorescence is measured by standard epifluorescent microscopy techniques. Plotting fluorescence versus compound concentration and using a four-parameter fit model generates an IC50 value for each compound.

Image-analysis is used to generate quantitative data from these assays that can be fit to a four-parameter sigmoid curve to derive IC50 values. Substrates of the ubiquitin-proteasome system, such as p53, are monitored after tumor cell lines are incubated with compounds for several hours. Accumulation of these proteins indicates an inhibition of proteasome-mediated degradation. Accumulation of lysine-48 chain linkage of poly-ubiquitin is also monitored by immunofluorescence as an indicator of ubiquitin-proteasome system inhibition. Both LC3 and SQSTM1 are mediators of autophagy. The localization and amounts of these proteins are monitored by immunofluorescence and report on the activity and inhibition of autophagy in response to p97 inhibition.

### Cultured Cancer Cell Assay

Anti-tumor effects are monitored in cultured cancer cells after several days of compound treatment. The cell titer glo assay (Promega) measures the amount of ATP present as a proxy for cellular viability. Cellular counting is done using high-content microscopy followed by image analysis. A hanging drop 3D-culture system (3D Biomatrix) is used followed by cell titer glo to measure growth in a tumor-like environment.

### Absorption Assay

The ability of compounds to be absorbed from the lumen of the gastrointestinal tract after oral administration was assessed by measuring their permeability through Caco-2 cell monolayers. SunD, et al., Curr. Opin. Drug Discov. Develop. (2004) 75. The in vitro permeability of compound (2 μM in Kreb's buffer or HBSS buffer with n=2) was determined using 21-day old Caco-2 cell monolayers. The permeation coefficient was determined for both Apical to Basolateral (A to B) and Basolateral to Apical (B to A) after 120 min at 37° C. The efflux ratio was calculated based on the ratio of permeation coefficient of B to A vs. A to B to determine the potential of compound as substrate for efflux pump (e.g. Pgp). The protocol for this Caco-2 assay and the corresponding detailed description are provided in the following experimental section.

### Metabolic Stability Assay

Metabolic stability of compounds can be assessed by measuring their half lives in liver microsomal preparations. Roserts, Sa, et al., Xenobiotica (2001) 37:557. Compounds are applied to a preparation of mouse liver microsomes in the presence of NADPH and their half lives are determined by measuring the rate of disappearance of the compounds from the preparation by determining the concentration at 0, 15, 30 and 60 minutes using LCMS/MS. The protocol for determining metabolic stability in a mouse liver assay and the corresponding detailed description are provided in the following experimental section.

### Nonspecific Binding Assay

Many compounds are known to bind nonspecifically to proteins found in high abundance in the plasma. The fraction of unbound drug (free fraction) is available for interaction with targets found in tissues. Banker, M. J. et al., Curr. Drug Metab. (2008) 9:854. The ability of compounds to escape a chamber containing blood plasma to a chamber containing only buffer can be assessed by measuring the concentration that appears in the buffer chamber and the concentration that remains in the plasma chamber. These measurements can be used to determine the fraction of compound bound to plasma proteins and its free fraction (100-percent bound to plasma proteins). The protocol for determining non-specific protein binding in a plasma protein binding assay and the corresponding detailed description are provided in the following experimental section.

### Mechanism of Action and Medical Treatment

In certain embodiments, the invention is directed to methods of inhibiting p97. The tricyclic compounds for use in the methods disclosed herein bind to the active site of p97, e.g., noncovalently or covalently. In certain such embodiments, the covalent binding may be reversible or irreversible.

The compounds of the invention and their pharmaceutical compositions are capable of acting as “inhibitors” of p97 which means that they are capable of blocking or reducing the activity of an enzyme, for example, inhibition of various activities of p97. An inhibitor can act with competitive, uncompetitive, or noncompetitive inhibition. An inhibitor can bind reversibly or irreversibly, and therefore the term includes compounds that are suicide the enzyme, or it can cause a conformational change elsewhere on the enzyme.

The compounds of the invention and their pharmaceutical compositions function as therapeutic agents in that they are capable of preventing, ameliorating, modifying and/or affecting a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The ability to prevent, ameliorate, modify and/or affect in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The compounds of the invention and their pharmaceutical compositions are capable of functioning prophylacticly and/or therapeutically and include administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The compounds of the invention and their pharmaceutical compositions are capable of prophylactic and/or therapeutic treatments. If a compound or pharmaceutical composition is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof). As used herein, the term “treating” or “treatment” includes reversing, reducing, or arresting the symptoms, clinical signs, and underlying pathology of a condition in manner to improve or stabilize a subject's condition.

The compounds of the invention and their pharmaceutical compositions can be administered in “therapeutically effective amounts” with respect to the subject method of treatment. The therapeutically effective amount is an amount of the compound(s) in a pharmaceutical composition which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

### Administration

Compounds prepared as described herein can be administered in various forms, depending on the disorder to be treated and the age, condition, and body weight of the patient, as is well known in the art. For example, where the compounds are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means, and if desired, the active ingredient may be mixed with any conventional additive or excipient, such as a binder, a disintegrating agent a lubricant, a corrigent, a solubilizing agent, a suspension aid, an emulsifying agent, a coating agent, a cyclodextrin, and/or a buffer. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable carrier” is a pharmaceutically acceptable material, composition, or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted (3-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound of the invention as an active ingredient. A composition may also be administered as a bolus, electuary, or paste.

In solid dosage form for oral administration (capsules, tablets, pills, dragees, powders, granules, and the like), a compound of the invention is mixed with one or more pharmaceutically acceptable carriers, such as sodium citrate or dicalcium phosphate, and/or any of the following:
(1) fillers or extenders, such as starches, cyclodextrins, lactose, sucrose, glucose, mannitol, and/or silicic acid;
(2) binders, such as, for example, carboxymethylcellulose, alginates, gelatin, polyvinyl pyrrolidone, sucrose, and/or acacia;
(3) humectants, such as glycerol;
(4) disintegrating agents, such as agar-agar, calcium carbonate, potato or tapioca starch, alginic acid, certain silicates, and sodium carbonate;
(5) solution retarding agents, such as paraffin;
(6) absorption accelerators, such as quaternary ammonium compounds;
(7) wetting agents, such as, for example, acetyl alcohol and glycerol monostearate;
(8) absorbents, such as kaolin and bentonite clay;
(9) lubricants, such a talc, calcium stearate, magnesium stearate, solid polyethylene glycols, sodium lauryl sulfate, and mixtures thereof; and
(10) coloring agents.
   In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered inhibitor(s) moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner.

Examples of embedding compositions which can be used include polymeric substances and waxes. A compound of the invention can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active inhibitor(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more inhibitor(s) with one or more suitable nonirritating excipients or carriers comprising, for example, cocoa butter, polyethylene glycol, a suppository wax or a salicylate, which is solid at room temperature, but liquid at body temperature and, therefore, will melt in the rectum or vaginal cavity and release the active agent.

Formulations which are suitable for vaginal administration also include pessaries, tampons, creams, gels, pastes, foams, or spray formulations containing such carriers as are known in the art to be appropriate.

Dosage forms for the topical or transdermal administration of an inhibitor(s) include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of the invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound of the invention can be alternatively administered by aerosol. This is accomplished by preparing an aqueous aerosol, liposomal preparation, or solid particles containing the composition. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of a compound of the invention together with conventional pharmaceutically acceptable carriers and stabilizers. The carriers and stabilizers vary with the requirements of the particular composition, but typically include nonionic surfactants (Tweens, Pluronics, sorbitan esters, lecithin, Cremophors), pharmaceutically acceptable co-solvents such as polyethylene glycol, innocuous proteins like serum albumin, oleic acid, amino acids such as glycine, buffers, salts, sugars, or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the inhibitor(s) across the skin. The rate of such flux can be controlled by either providing a rate controlling membrane or dispersing the inhibitor(s) in a polymer matrix or gel.

Pharmaceutical compositions of this invention suitable for parenteral administration comprise one or more compounds of the invention in combination with one or more pharmaceutically acceptable sterile aqueous or nonaqueous solutions, dispersions, suspensions or emulsions, or sterile powders which may be reconstituted into sterile injectable solutions or dispersions just prior to isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a compound of the invention, it is desirable to slow the absorption of the compound from subcutaneous or intramuscular injection. For example, delayed absorption of a parenterally administered drug form is accomplished by dissolving or suspending the drug in an oil vehicle.

Injectable depot forms are made by forming microencapsule matrices of inhibitor(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The pharmaceutical compositions may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The pharmaceutical compositions of the invention may be “systemically administered” “administered systemically,” “peripherally administered” and “administered peripherally” meaning the administration of a ligand, drug, or other material other than directly into the central nervous system, such that it enters the patient's system and thus, is subject to metabolism and other like processes, for example, subcutaneous administration.

The compound(s) of the invention may be administered to humans and other animals for therapy by any suitable route of administration, including orally, nasally, as by, for example, a spray, rectally, intravaginally, parenterally, intracistemally, and topically, as by powders, ointments or drops, including buccally and sublingually.

Regardless of the route of administration selected, the compound(s) of the invention, which may be used in a suitable hydrated form, and/or the pharmaceutical compositions of the present invention, are formulated into pharmaceutically acceptable dosage forms by conventional methods known to those of skill in the art.

Actual dosage levels of the compound(s) of the invention in the pharmaceutical compositions of this invention may be varied so as to obtain an amount of the active ingredient which is effective to achieve the desired therapeutic response for a particular patient, composition, and mode of administration, without being toxic to the patient.

The concentration of a compound of the invention in a pharmaceutically acceptable mixture will vary depending on several factors, including the dosage of the compound to be administered, the pharmacokinetic characteristics of the compound(s) employed, and the route of administration.

In general, the compositions of this invention may be provided in an aqueous solution containing about 0.1-10% w/v of a compound disclosed herein, among other substances, for parenteral administration. Typical dose ranges are from about 0.01 to about 50 mg/kg of body weight per day, given in 1-4 divided doses. Each divided dose may contain the same or different compounds of the invention. The dosage will be an effective amount depending on several factors including the overall health of a patient, and the formulation and route of administration of the selected compound(s).

Another aspect of the invention provides a conjoint therapy wherein one or more other therapeutic agents are administered with the compounds and compositions of the invention. Such conjoint treatment will achieve the same or similar treatment accounting for the additive effects of the conjoined therapeutic agents other than the compounds of the invention.

In certain embodiments, a compound of the invention is conjointly administered with one or more proteasome inhibitor(s). In certain embodiments, a compound of the invention is conjointly administered with a chemotherapeutic. Suitable chemotherapeutics may include, natural products such as vinca alkaloids (i.e., vinblastine, vincristine, and vinorelbine), paclitaxel, epidipodophyllotoxins (i.e., etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates (busulfan), nitrosoureas (carmustine (BCNU) and analogs, streptozocin), trazenes—dacarbazinine (DTIC); antiproliferative/antimitotic antimetabolites such as folic acid analogs (methotrexate), triazine, pyridine, pyrimidine analogs (fluorouracil, floxuridine, and cytarabine), purine analogs and related inhibitors (mercaptopurine, thioguanine, pentostatin and 2-chlorodeoxyadenosine); aromatase inhibitors carboplatin), procarbazine, hydroxyurea, mitotane, aminoglutethimide; hormones (i.e. estrogen) and hormone agonists such as leutinizing hormone releasing hormone (LHRH) agonists (goserelin, leuprolide and triptorelin). Other chemotherapeutic agents may include mechlorethamine, camptothecin, ifosfamide, tamoxifen, raloxifene, gemcitabine, navelbine, or any analog or derivative variant of the foregoing.

In certain embodiments, a compound of the invention is conjointly administered with a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fluclorinide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, paramethasone, prednicarbate, prednisolone, prednisolone 25-diethylaminoacetate, prednisolone, sodium phosphate, prednisone, prednival, prednylidene, rimexolone, tixocortol, triamcinolone, triamcinolone acetonide, triamcinolone benetonide, triamcinolone hexacetonide, and salts and/or derivatives thereof.

In certain embodiments, a compound of the invention is conjointly administered with an immunotherapeutic agent. Suitable immunotherapeutic agents may include, but are not limited to, cyclosporine, thalidomide, and monoclonal antibodies. The monoclonal antibodies can be either naked or conjugated such as rituximab, tositumomab, alemtuzumab, epratuzumab, ibritumomab tiuxetan, gemtuzamab ozogamicin, bevacizumab, cetuximab, erlotinib and trastuzumab.

### Treatment of Cancer

Exemplary forms of cancer which may be treated by the methods of the invention include, but are not limited to, prostate cancer, bladder cancer, lung cancer (including either small cell or non-small cell cancer), colon cancer, kidney cancer, liver cancer, breast cancer, cervical cancer, endometrial or other uterine cancer, ovarian cancer, testicular cancer, cancer of the penis, cancer of the vagina, cancer of the urethra, gall bladder cancer, esophageal cancer, or pancreatic cancer.

Additional exemplary forms of cancer which may be treated by the methods of the invention include, but are not limited to, cancer of skeletal or smooth muscle, stomach cancer, cancer of the small intestine, cancer of the salivary gland, anal cancer, rectal cancer, thyroid cancer, parathyroid cancer, pituitary cancer, and nasopharyngeal cancer.

The compounds of the present invention and their salts and solvates, thereof, may be employed alone or in combination with other therapeutic agents for the treatment of the diseases or conditions associated with inappropriate P97 activity.

Additional diseases that can be treated according to the methods of the invention include in addition to cancer, auto-immune disorders, metabolic diseases, infection diseases, neurological diseases, graft versus host disease and other hereditary diseases outlined here: abeta-lipoproteinema, acerulopasminemia, alpha-1-antichymotrypsin (ACT) deficiency, aspartylglucosaminuria, autosomal dominant retinitis pigmentosa, brugada syndrome, Charcot-Marie-Tooth syndrome, congenital adrenal hyperplasia, congenital chloride diarrhea, congenital hypothyroidism, congenital long QT syndrome, congenital nephritic syndrome, congenital sucrase-isomaltase deficiency, Crigler-Najjar type II, cystic fibrosis, diabetes mellitus, diastrophic displasia, Dubin-Johnson syndrome, Fabri disease, familial chylomicronemia, familial glucocorticoid deficiency, familial hypercholesterolemia, Gaucher disease, heavy chain disease, hereditary emphysema, hereditary emphysema with liver injury, hereditary hemochromatosis, hereditary hypofibrinogenemia, hereditary myeloperoxidase, hereditary spherocytosis, hirschprung disease, hypogonadotropic hypogonadism, infantile systemic hyalinosis, infentile neuronal ceroid lipofuscinosis, laron syndrome, liver failure, lupus erythematosus, marfan syndrome, medullary cystic kidney disease, familial juvenile hyperuricemic nephropathy, Menkes disease, nephrogenic diabetes, neurohypophyseal diabetes insipidus, oculocutaneous albinism, osteogenesis imperfect, Pelizaeus-Merzbacher disease, Pendred syndrome, persistent hyperinsulinemic hypoglycemia of infancy, primary hypothyroidism, Protein C deficiency, pseudoachondropla with multiple epiphyseal dysplasia, severe congenital neutropenia, Stargardt-like macular dystrophy, steroid-resistant nephrotic syndrome, Tay-Sachs, Type I hereditary angioedema, tyroxine binding globulin deficiency, von Willebrand disease type IIA, X-linked Charot-Marie-Tooth disease, X-linked hypophosphatemia, Alzheimer disease autosomal recessive juvenile parkinsonism, combined factors V and VIII deficiency, cranio-lenticulo-sutural dysplasia, hypotonia and dysmorphism, inclusion body myopathy Paget's disease of the bone and fronto-temporal dementia (IBMPFD), lipid absorption disorders, Marinesco-Sjoegren syndrome, Parkinson, polycystic liver disease, spondylo-epiphyseal dysplasia tarda, Walcott-Rallison syndrome and Lou Gehrig's disease (ALS).

In various embodiments, compounds of the invention may be used to treat neoplastic growth, angiogenesis, infection, inflammation, immune-related diseases, ischemia and reperfusion injury, multiple sclerosis, rheumatoid arthritis, neurodegenerative conditions, or psoriasis.

Neoplastic growth may include cancer. Suitably, the present invention relates to a method for treating or lessening the severity of a cancer selected from: brain (gliomas), glioblastomas, breast, Wilm's tumor, Ewing's sarcoma, rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid, lymphoblastic T cell leukemia, chronic myelogenous leukemia, chronic lymphocytic leukemia, Hairy-cell leukemia, acute lymphoblastic leukemia, acute myelogenous leukemia, chronic neutrophilic leukemia, acute lymphoblastic T cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

In various embodiments, the cancer is selected from brain cancer (gliomas), glioblastomas, breast cancer, colon cancer, head and neck cancer, kidney cancer, lung cancer, liver cancer, melanoma, ovarian cancer, pancreatic cancer, prostate cancer, sarcoma and thyroid cancer.

In various embodiments, the cancer to be treated is associated with the proteasome. See Voorhees et al., The Proteasome as a Target for Cancer Therapy, Clinical Cancer Research, vol. 9, 6316-6325, December 2003, incorporated by reference in its entirety. In various embodiments, the cancer is associated with a particular target, such as NFkB, p44/42 MAPK, P-gp, TopI, TopIIalpha.

In various embodiments, the cancer is a solid tumor. In various embodiments, the cancer is selected from multiple myeloma, metastatic breast cancer, non-small cell lung cancer, prostate cancer, advanced colorectal cancer, ovarian or primary peritoneal carcinoma, hormone refractory prostate cancer, squamous cell carcinoma of the head and neck, metastatic pancreatic adenocarcinoma, gastroesophageal junction or stomach, or non-Hodgkin's lymphoma.

A method of using the compounds described herein for treating a disorder characterized by an inappropriate level of proteasome activity, or in which a reduction of the normal level of proteasome activity yields a clinical benefit. This disorder can include cancer or immune disorders characterized by excessive cell proliferation or cellular signaling. Among cancers, this includes human cancers that overexpress c-Myc or express an oncogenic form of the K-Ras protein.

Neurodegenerative diseases and conditions may include without limitation stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, ALS, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis). Compounds of the invention may be used to treat Alzheimer's disease, including administering to a subject an effective amount of an agent or composition (e.g., pharmaceutical composition) disclosed herein.

Compounds of the invention may be used to treat cachexia and muscle-wasting diseases. Compounds of the invention may be used to treat such conditions wherein the condition is related to cancer, chronic infectious diseases, fever, muscle disuse (atrophy) and denervation, nerve injury, fasting, renal failure associated with acidosis, diabetes, and hepatic failure.

Compounds of the invention can be used to treat hyperproliferative conditions such as diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders). The treatment of burn victims is often hampered by fibrosis, thus, an additional embodiment of the application is the topical or systemic administration of the inhibitors to treat burns. Wound closure following surgery is often associated with disfiguring scars, which may be prevented by inhibition of fibrosis. Thus, in certain embodiments, the application relates to a method for the prevention or reduction of scarring.

Compounds of the invention can be used to treat ischemic conditions or reperfusion injury for example acute coronary syndrome (vulnerable plaques), arterial occlusive disease (cardiac, cerebral, peripheral arterial and vascular occlusions), atherosclerosis (coronary sclerosis, coronary artery disease), infarctions, heart failure, pancreatitis, myocardial hypertrophy, stenosis, and restenosis.

Compounds of the invention can be used for the inhibition of TNFalpha to prevent and/or treat septic shock.

Compounds of the invention can be used for inhibiting antigen presentation in a cell, including exposing the cell to an agent described herein. A compound of the invention may be used to treat immune-related conditions such as allergy, asthma, organ/tissue rejection (graft-versus-host disease), and auto-immune diseases, including, but not limited to, lupus, rheumatoid arthritis, psoriasis, multiple sclerosis, and inflammatory bowel diseases (such as ulcerative colitis and Crohn's disease). Thus, a further embodiment is a method for modulating the immune system of a subject (e.g., inhibiting transplant rejection, allergies, auto-immune diseases, and asthma), including administering to the subject an effective amount of a compound of the invention.

Compounds of the invention can be used in methods for altering the repertoire of antigenic peptides produced by the proteasome or other protein assembly with multicatalytic activity.

Compounds of the invention can be used in methods for inhibiting IKB-alpha degradation, including contacting the cell with an agent identified herein. A further embodiment is a method for reducing the cellular content of NF—KB in a cell, muscle, organ, or subject, including contacting the cell, muscle, organ, or subject with a compound of the invention.

Compounds of the invention can be used in methods for affecting cyclin-dependent eukaryotic cell cycles. Compounds of the invention can be used in methods for treating a proliferative disease in a subject (e.g., cancer, psoriasis, or restenosis). Compounds of the invention can be used for treating cyclin-related inflammation in a subject.

One embodiment is a method for treating p53-related apoptosis, including administering to a subject an effective amount of a compound of the invention.

In another embodiment, the agents of the present application are useful for the treatment of a parasitic infection, such as infections caused by protozoan parasites. In certain such embodiments, the agents are useful for the treatment of parasitic infections in humans caused by a protozoan parasite selected from Plasmodium sps., Trypanosoma sps., Leishmania sps., Pneumocystis carinii, Toxoplasma gondii, Entamoeba histolytica, Entamoeba invadens, and Giardia lamblia. In certain embodiments, the agents are useful for the treatment of parasitic infections in animals and livestock caused by a protozoan parasite selected from Plasmodium hermani, Cryptosporidium sps., Echinococcus granulosus, Eimeria tenella, Sarcocystis neurona, and Neurospora crassa. Other compounds useful as proteasome inhibitors in the treatment of parasitic diseases are described in WO 98/10779, which is incorporated herein in its entirety.

In particular, the methods of treatment include inhibiting, arresting, ameliorating, minimizing and/or eliminating malconditions associated with the inability of cells to metabolize, degrade or otherwise remove ubiquitin tagged proteins and peptides because the tag has been cleaved, degraded, removed or otherwise rendered disfunctional as a result of P97 metalloprotease domain activity. Included are methods in which a human disorder characterized by abnormal regulatory peptide degradation resulting in excessive cell proliferation or cell signaling. The methods are directed to administration of an effective amount of a compound or pharmaceutical formulation disclosed above so that the abnormal regulatory peptide degradation is ameliorated, reduced or inhibited. In particular, the human disorders include a cancer or immune disorder, a cancer resulting from overexpression of c-Myc or expression of an oncogenic form of the K-Ras protein. The methods also include inhibition or amelioration of P97 metalloprotease domain activity in a human patient suffering from abnormal P97 metalloprotease domain activity on ubiquitin modified proteins. As described above, these methods involve administering to the patient an effective amount of a compound or pharmaceutical formulation disclosed above so that the abnormal P97 metalloprotease domain activity is ameliorated, reduced or inhibited.

### Additional Embodiments of the Compounds of the Invention

Additional embodiments of the compounds of the invention include the following variations of the substituents Y and R¹ to R⁶. Each of these variations can be combined with any other variation as is appropriate for the final structure of the tricyclic fused pyrimidine compound desired to form a full tricyclic compound of the invention. For R¹, hydrogen, short chain aliphatic groups, short chain acyl and sulfonyl groups listed below are preferred. For R² only hydrogen and methyl are preferred. For R³ and R⁶ hydrogen and methyl are preferred. For R⁴ aliphatic and functional groups listed below are preferred with aliphatic groups being more preferred and hydrogen being especially preferred. For R⁵ the aliphatice groups listed below are preferred. For Y, only the functional groups listed below apply. For Y and X, variation 25, which adds a peptide monomer, dimer, trimer or tetramer to Formula I, is a notable additional embodiment.

The number designations for the carbons include all integers between the lowest and highest number. Individual numbers of carbon atoms separate and distinct from other numbers of the same group are also included. For example for an alkyl of 1 to 6 carbons, an alkyl group of 1, 2, 3, 4, 5 or 6 carbons is included as well as each individual number designation separate and distinct from other number designations so that an alkyl of 1 to 6 carbons includes separately, methyl, ethyl, propyl, butyl, pentyl and hexyl.
1) Linear, branched or cyclic alkyl of 1 to 6 carbons,
2) Linear, branched or cyclic alkoxy of 1 to 6 carbons,
3) Amine and aminoalkyl (eg, —NHR and —NR₂)
4) Carboxylic acid,
5) Carboxylic ester wherein the alkoxy group of the ester is from 1 to 6 branched or straight carbons or the alcohol esterifying group is phenoxy,
6) Linear, branched or cyclic alkylenyl carboxylic acid or ester of 2 to 7 carbons in the alkylenyl group and 1 to 6 branched or straight carbons in the ester group, for example a linear alkylenyl carboxylic acid of 2 carbons in the alkylenyl group is —CH₂CH₂COOH,
7) Branched or straight alkylenyl amine of 1 to 6 carbons (eg, —R—NH₂),
8) Linear, branched or cyclic perfluoroalkyl of 1 to 6 carbons,
9) Linear, branched or cyclic trifluoroalkyl of 1 to 6 carbons wherein the trifluoro group is on the terminating or end carbon,
10) Hydroxyl,
11) Linear, branched or cyclic alkylenyl hydroxyl of 1 to 6 carbons,
12) Carboxamide eg., —CONH₂,
13) Linear, branched or cyclic alkylenylcarboxamide of 1 to 6 carbons in the alkylenyl group,
14) N-substituted carboxamide, wherein the N substituent is an aryl group, heteroaryl group or heterocycle group as defined in the DEFINITIONS section, eg., —CONHAr or —CONHHet,
15) N-substituted carboxamide wherein the N substituent is an alkaryl group, an alkheteroaryl group or an alkheterocycle group as defined in the DEFINITIONS section, and wherein the “alk” group is alinear, branched or cyclic alkylenyl group of 1 to 6 carbons, eg., —CONH—R—Ar or —CONH—R-Het,
16) N-substituted carboxamide wherein the N substituent is a branched or straight alkyl group of 1 to 10 carbons, the polyfluorinated version thereof, or a substituted version thereof, eg., —CONH—R, wherein the substituent of the alkyl group is halogen, cyano, carboxyl, ester of 1 to 6 branched or straight chain carbons in the alkoxy or phenoxy portion, carboxamide, sulfoxamide, alkoxy of 1 to 6 carbons, urea, carbamate of 1 to 10 carbons, amine, mono or dialkyl amine having from 1 to 6 carbons in the alkyl group with the alkyl group being straight or branched, hydroxyalkyl of 1 to 10 branched or straight chain carbons or a cycloalkyl group as defined in the DEFINITIONS section,
17) Aminocarbonylalkyl, eg., —NHCOR, wherein R is a linear, branched or cyclic alkyl of 1 to 6 carbons,
18) Alkyleneaminocarbonylalkyl, eg., —RNHCOR, wherein the alkylenyl is linear, branched or cyclic and is 1 to 6 carbons and the alkyl is linear, branched or cyclic and is 1 to 6 carbons,
19) Sulfonamide, eg., SO₂NH₂,
20) Linear, branched or cyclic alkylenylsulfonamide of 1 to 6 carbons in the alkylenyl group,
21) N-substituted sulfonamide, wherein the N substituent is an aryl group, heteroaryl group or heterocycle group as defined in the DEFINITIONS section, eg., —SO₂NHAr or —SO₂NHHet,
22) A heterocyclic system comprised of one or more of the following: an azetidine or substituted azetidine attached as any of the R groups, pyrrolidine or substituted pyrrolidine attached as any of the R groups, piperidine or substituted piperidine attached as any of the R groups, a piperazine or substituted piperazine attached as any of the R groups, a morphorpline or substituted morpholine attached as any of the R groups, with the proviso that when the R group is attached to a nitrogen, the resulting heterocyclic system results in a stable R—N configuration,
23) Preferred aryl, heteroaryl and heterocycle groups for 14 and 15 include phenyl, halogen substituted phenyl, aminophenyl, benzoic acid, tolyl, xylyl, anisolyl, trifluoromethylphenyl, benzyl, tetrahydrofuran, pyrrolidinyl, tetrahydronaphthalene, cyclohexyl or alkyl substituted cyclohexyl with the alkyl group having 1 to 6 carbons, cyclohexyl or alkyl substituted cyclohexyl with the alkyl group having 1 to 6 carbons, cyclopentyl or alkyl substituted cyclopentyl with the alkyl group having 1 to 6 carbons, pyrazolyl, imidazolyl, piperidinyl, piperazinyl, pyrimidinyl, morpholinyl, pyrrolyl, thiophenyl, substituted versions of any of the foregoing aryl, heteroaryl or heterocycle groups wherein the chemical substituent is halogen, cyano, carboxyl, ester of 1 to 10 branched or straight chain carbons in the alkoxy or phenoxy portion, amine, carboxamide, sulfoxamide, urea, carbamate of 1 to 10 carbons, hydroxyl, thiol, alkoxy, anisolyl, phenyl, benzyl or a cycloalkyl group as defined in the DEFINITIONS section,
24) Derivatives of 14-21 wherein the N of the carboxamide, aminocarbonyl or sulfonamide has a second substituent and the second substituent is a branched or straight chain alkyl of 1 to 6 carbons,
25) An N-substituted carboxamide or sulfonamide wherein the N substituent is a mono, di, tri or tetra amino acid, and substituted amine groups wherein the substituent is a residue of a mono, di, tri or tetra amino acid, the amino acid moieties include glycinyl, alaninyl, leucinyl, valinyl, phenylalaninyl, lysinyl, argininyl, histidinyl, serinyl, aspariginyl, glutaminyl, aspartic, glutamic such that the amino acid moieties may be combined in any combination of two, three or four moieties including but not limited to a tetramer of four different moieties, a tetramer of two and two different moieties, a tetramer of three of one moiety and one of a different moiety, a trimer of two of one moiety and one of another moiety or a trimer of three different moieties, a dimer of two different moieties of of the same moiety, and a monomer of any of the designated moieties. The nitrogen of an amino acid moiety may serve as the nitrogen of the carboxyamide group or may serve as the nitrogen of an amine substituent of Formula I. The C-terminus of the amino acid monomer, dimer or trimer may be a carboxylic acid or a carboxamide. The order of amino acid moieties in the tetramer, trimer or dimer may be any order.
26) Any of the substituents designated by items 1-3, 5-9, 11, 13-25 which additionally includes any functional group selected from F, Cl, Br, I, OR′, B(OH)₂, B(OMe or Et)₂, OC(O)N(R′)₂, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl.
27) In addition to the groups of substituents set forth in 1 through 26 above, each individual substituent and individual combination is included separately and individually as if it were individually recited.
28) Additional embodiments of the compounds of the invention further include each individual compound listed on the compound List above.
29) Optional Hydrogen.

### Synthetic and Biological Examples

The following describes the preparation of representative compounds of the invention in greater detail. The following examples are offered for illustrative purposes, and are not intended to limit the invention in any manner. Those of skill in the art will readily recognize a variety of noncritical parameters which can be changed or modified to yield essentially the same results.

Those skilled in the art will recognize, or be able to ascertain using no more than routine experimentation, numerous equivalents to the syntheses of the compounds and methods of use thereof described herein. Although certain exemplary embodiments are depicted and described herein, it will be appreciated that compound of the invention can be prepared according to the methods generally available to one of ordinary skill in the art. All of the above-cited references and publications are hereby incorporated by reference.

Unless otherwise noted, all solvents, chemicals, and reagents were obtained commercially and used without purification. The ¹H NMR spectra were obtained in CDCl₃, d₆-DMSO, CD₃OD, or d₆-acetone at 25° C. at 300 MHz on an OXFORD (Varian) spectrometer with chemical shift (δ, ppm) reported relative to TMS as an internal standard. HPLC-MS chromatograms and mass spectra were obtained with Shimadzu LC-MS-2020 system. The prep-HPLC instruments used to purify some compounds were either a Gilson GX-281 (Gilson) or a P230 Preparative Gradient System (Elite). Preparative chira HPLC separations were performed using an Elite P230 Preparative Gradient System, a Thar Prep-80 or Thar SFC X-5. Reactions using microwave irradiation were performed on a CEM Discover SP instrument.

### Synthetic Examples

### Example 01

### Synthesis of 1-(4-(benzylamino)-6,8,9,10,11,11a-hexahydro-5H-pyrimido[4,5-a]quinolizin-2-yl)-2-methyl-1H-indole-4-carboxamide (001)

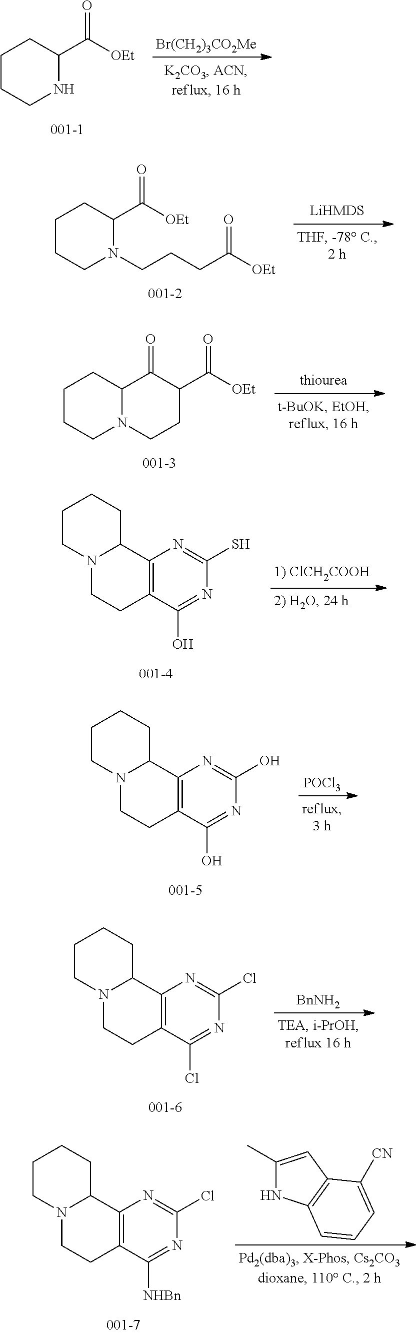

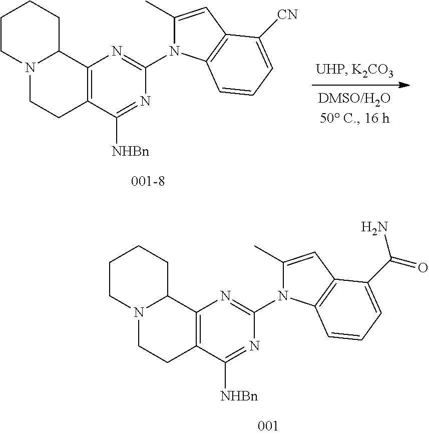

### Ethyl 1-(4-ethoxy-4-oxo-butyl)piperidine-2-carboxylate (001-2)

To a 25° C. solution of ethyl piperidine-2-carboxylate (10 g, 63.61 mmol) (001-1) and ethyl 4-bromobutanoate (12.41 g, 63.61 mmol) in MeCN (100 mL) was added K₂CO₃ (13.19 g, 95.41 mmol). The reaction was stirred at 80° C. for 16 hr under nitrogen. LCMS showed the reaction was completed. The reaction was concentrated and added with water (30 mL). The mixture was extracted with ethyl acetate (50 mL×3). The combined organic layers were washed with brine (30 mL), dried over Na₂SO₄, and evaporated in vacuo. The residue was purified by column chromatography (silica gel, petroleum ether, ethyl acetate) to give 15.5 g (89.8%) of ethyl 1-(4-ethoxy-4-oxo-butyl)piperidine-2-carboxylate as white oil. LRMS (M+H⁺) m/z: Calcd: 272; Found: 272.2.

### Ethyl 1-oxooctahydro-1H-quinolizine-2-carboxylate (001-3)

To a solution of ethyl 1-(4-ethoxy-4-oxo-butyl)piperidine-2-carboxylate (14.5 g, 53.44 mmol) in THF (150 mL) was added LiHMDS (19.56 g, 106.87 mmol) dropwise at −78° C. under nitrogen. The reaction was stirred for 2 hr at 25° C. LCMS showed the reaction was completed. The reaction was added with water (20 mL) and extracted with ethyl acetate (100 mL×3). The combined organic layers were washed with brine (30 mL), dried over Na₂SO₄, and evaporated in vacuo. The residue was purified by column chromatography (silica gel, petroleum ether, ethyl acetate) to give 11.5 g (95.5%) of ethyl 1-oxooctahydro-1H-quinolizine-2-carboxylate. LRMS (M+H⁺) m/z: Calcd: 226; Found: 226.2.

### 2-Mercapto-6,8,9,10,11,11a-hexahydro-5H-pyrimido[4,5-a]quinolizin-4-ol (001-4)

To a 25° C. solution of ethyl 1-oxooctahydro-1H-quinolizine-2-carboxylate (4 g, 17.76 mmol) and thiourea (2.03 g, 26.63 mmol) in EtOH (50 mL) was added t-BuOK (3.98 g, 35.51 mmol). The reaction was stirred at 90° C. for 16 hr under nitrogen. LCMS showed the reaction was completed. The reaction was then quenched with water (50 mL) and HCl aqueous solution (20 mL, 1 N). The precipitated solids were collected and dried to afford 2-mercapto-6,8,9,10,11,11a-hexahydro-5H-pyrimido[4,5-a]quinolizin-4-ol (4 g, 94.9%) as white powder. LRMS (M+H⁺) m/z: Calcd: 238; Found: 238.1.

### 6,8,9,10,11,11a-Hexahydro-5H-pyrimido[4,5-a]quinolizine-2,4-diol (001-5)

To a 25° C. solution of 2-mercapto-6,8,9,10,11,11a-hexahydro-5H-pyrimido[4,5-a]quinolizin-4-ol (4 g, 20 mmol) in H₂O (40 mL) was added 2-chloroacetic acid (4.78 g, 50.56 mmol). The reaction was stirred at 100° C. for 24 hr. LCMS showed the reaction was completed. The reaction was concentrated to yield crude 6,8,9,10,11,11a-hexahydro-5H-pyrimido[4,5-a]quinolizine-2,4-diol (9 g, 96.5%) as white powder. LCMS (M+H⁺) m/z: Calcd: 222; Found: 222.2.

### 2,4-Dichloro-6,8,9,10,11,11a-hexahydro-5H-pyrimido[4,5-a]quinolizine (001-6)

A solution of 6,8,9,10,11,11a-hexahydro-5H-pyrimido[4,5-a]quinolizine-2,4-diol (6 g, 27.12 mmol) in POCl₃ (110 mL) was stirred at 120° C. for 3 hr. LCMS showed the reaction was completed. The reaction was concentrated and diluted with water (30 mL) at 0° C., and extracted with ethyl acetate (100 mL×3). The combined organic layers were washed with brine (30 mL), dried over Na₂SO₄. The residue was concentrated to give 2,4-dichloro-6,8,9,10,11,11a-hexahydro-5H-pyrimido[4,5-a]quinolizine (0.8 g, 11.4%) as red powder. LRMS (M+H⁺) m/z: Calcd: 258; Found: 258.2.

### N-Benzyl-2-chloro-6,8,9,10,11,11a-hexahydro-5H-pyrimido[4,5-a]quinolizin-4-amine (001-7)

To a 25° C. solution of 2,4-dichloro-6,8,9,10,11,11a-hexahydro-5H-pyrimido[4,5-a]quinolizine (400 mg, 1.55 mmol) and phenylmethanamine (166 mg, 1.55 mmol) in iPrOH (10 mL) was added TEA (470 mg, 4.65 mmol). The reaction was stirred at 60° C. for 16 hr. LCMS showed the reaction was completed. The reaction was evaporated in vacuo. The residue was purified by column chromatography (silica gel, petroleum ether, ethyl acetate) to give N-benzyl-2-chloro-6,8,9,10,11,11a-hexahydro-5H-pyrimido[4,5-a]quinolizin-4-amine (170 mg, 33.4%) as yellow powder. LCMS (M+H⁺) m/z: Calcd: 329; Found: 329.2.

### 1-[4-(Benzylamino)-6,8,9,10,11,11a-hexahydro-5H-pyrimido[4,5-a]quinolizin-2-yl]-2-methyl-indole-4-carbonitrile (001-8)

To a 25° C. solution of N-benzyl-2-chloro-6,8,9,10,11,11a-hexahydro-5H-pyrimido[4,5-a]quinolizin-4-amine (150 mg, 0.46 mmol) and 2-methyl-1H-indole-4-carbonitrile (71 mg, 0.46 mmol) in dioxane (10 mL) was added Pd₂(dba)₃ (41 mg, 45 μmol), X-phos (43 mg, 0.09 mmol), Cs₂CO₃ (296 mg, 0.91 mmol). The reaction was stirred at 110° C. for 2 hr under nitrogen. LCMS showed the reaction was completed. The reaction was evaporated in vacuo. The residue was purified by column chromatography (silica gel, petroleum ether, ethyl acetate) to give 1-[4-(benzylamino)-6,8,9,10,11,11a-hexahydro-5H-pyrimido[4,5-a]quinolizin-2-yl]-2-methyl-indole-4-carbonitrile (150 mg, 73.3%) as yellow powder. LRMS (M+H⁺) m/z: Calcd: 449; Found: 449.3.

### 1-[4-(Benzylamino)-6,8,9,10,11,11a-hexahydro-5H-pyrimido[4,5-a]quinolizin-2-yl]-2-methyl-indole-4-carboxamide (001)

To a 25° C. solution of 1-[4-(benzylamino)-6,8,9,10,11,11a-hexahydro-5H-pyrimido[4,5-a]quinolizin-2-yl]-2-methyl-indole-4-carbonitrile (150 mg, 0.33 mmol) in DMSO (10 mL) and H₂O (1 mL) were added UHP (157 mg, 1.67 mmol) and K₂CO₃ (46 mg, 0.33 mmol). The reaction was stirred at 50° C. for 16 hr. LCMS showed the reaction was completed. The reaction was diluted with water (5 mL) and extracted with ethyl acetate (50 mL×3). The combined organic layers were washed with brine (30 mL), dried over Na₂SO₄. The residue was concentrated and purified by Prep-HPLC (formic acid) to give 8.2 mg (5.3%) of 1-[4-(benzylamino)-6,8,9,10,11,11a-hexahydro-5H-pyrimido[4,5-a]quinolizin-2-yl]-2-methyl-indole-4-carboxamide (8.2 mg, 5.3%) as yellow powder. LRMS (M+H⁺) m/z: Calcd: 467; Found: 467.3. ¹H NMR (400 MHz, DMSO-d6): δ 1.17-1.32 (m, 1H, CH) 1.41 (q, J=12.35 Hz, 1H, CH) 1.48-1.60 (m, 1H, CH) 1.60-1.70 (m, 1H, CH) 1.86 (d, J=12.35 Hz, 1H, CH) 2.18-2.31 (m, 1H, CH) 2.49 (br. s., 3H, CH₃) 2.53 (d, J=6.62 Hz, 2H, CH₂) 2.59-2.72 (m, 1H, CH) 2.97 (d, J=10.58 Hz, 2H, CH₂) 3.05 (dd, J=10.36, 5.95 Hz, 1H, CH) 4.57-4.73 (m, 2H, CH₂) 6.84 (s, 1H, Ar) 6.90 (t, J=7.94 Hz, 1H, Ar) 7.14-7.28 (m, 2H, Ar) 7.29-7.38 (m, 4H, Ar) 7.44 (d, J=7.50 Hz, 1H, NH) 7.71 (br. s., 1H, Ar) 7.77 (d, J=7.94 Hz, 2H, NH₂) 8.16 (s, 1H, Ar)

### Example 02

### Synthesis of 1-(4-(benzylamino)-5,6,8,9,10,10a-hexahydropyrimido[5,4-g]indolizin-2-yl)-2-methyl-1H-indole-4-carboxamide (002)

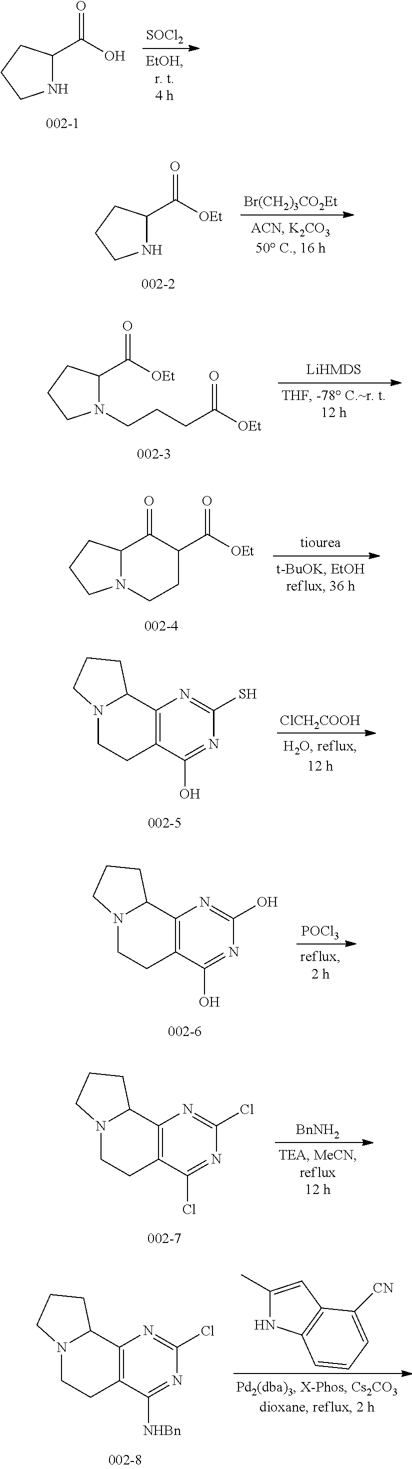

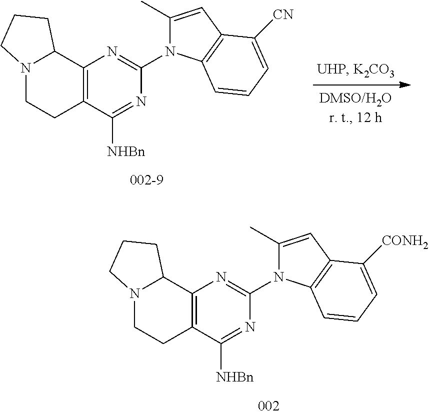

### Ethyl Pyrrolidine-2-carboxylate (002-2)

To a 15° C. solution of pyrrolidine-2-carboxylic acid (30 g, 260.57 mmol) in EtOH (400 mL) was added SOCl₂ (40 ml). The reaction was stirred for 4 hr. LCMS showed the reaction was completed. The reaction was concentrated in vacuo to give ethyl pyrrolidine-2-carboxylate (35 g, 84.4%) as white solid. LCMS (M+H⁺) m/z: Calcd: 144.09; Found: 144.2.

### Ethyl 1-(4-ethoxy-4-oxo-butyl)pyrrolidine-2-carboxylate (002-3)

A solution of ethyl pyrrolidine-2-carboxylate (20 g, 140 mmol), ethyl 4-bromobutanoate (20 ml, 140 mmol), K₂CO₃ (28.96 g, 209 mmol) in MeCN (500 mL) was stirred at 50° C. for 16 hr. TLC showed the reaction was completed. The reaction was added with water (100 mL) and extracted with ethyl acetate (100 mL×3). The combined organic layers were washed with brine (30 mL), dried over Na₂SO₄, and evaporated in vacuo. The residue was purified by column chromatography (silica gel, petroleum ether, ethyl acetate) to give ethyl 1-(4-ethoxy-4-oxo-butyl)pyrrolidine-2-carboxylate (25 g, 55.6%) as oil.

### Ethyl 8-oxo-2,3,5,6,7,8a-hexahydro-1H-indolizine-7-carboxylate (002-4)

A solution of ethyl 1-(4-ethoxy-4-oxo-butyl)pyrrolidine-2-carboxylate (25 g, 97.15 mmol) in THF (800 mL) was added LiHMDS (1M, 146 mL) dropwise at −78° C. under nitrogen. The reaction was slowly warmed to 15° C., and stirred for 12 hr. LCMS showed the reaction was completed. The reaction was quenched by H₂O (500 mL). The mixture was extracted by DCM (200 mL×3). The combined organic layers were dried over Na₂SO₄, concentrated in vacuo to give ethyl 8-oxo-2,3,5,6,7,8a-hexahydro-1H-indolizine-7-carboxylate (12 g, 46.8%) as red oil. LCMS (M+H⁺) m/z: Calcd: 212.12; Found: 212.2.

### 2-Mercapto-5,6,8,9,10,10a-hexahydropyrimido[5,4-g]indolizin-4-ol (002-5)

A solution of ethyl 8-oxo-2,3,5,6,7,8a-hexahydro-1H-indolizine-7-carboxylate (12 g, 56.8 mmol), thiourea (8.65 g, 113.61 mmol), t-BuOK (12.75 g, 113.61 mmol) in EtOH (50 mL) was stirred 80° C. for 36 hr. LCMS showed the reaction was completed. The reaction was concentrated in vacuo and water (20 mL). Aqueous HCl solution (1N) was added to adjust pH to 4˜5. The mixture was stirred at 0° C. for 10 min, the precipitated solids were collected and dried to give 2-mercapto-5,6,8,9,10,10a-hexahydropyrimido[5,4-g]indolizin-4-ol (2 g, 14.2%) as white solid. LCMS (M+H⁺) m/z: Calcd: 224.08; Found: 224.1.

### 5,6,8,9,10,10a-Hexahydropyrimido[5,4-g]indolizine-2,4-diol (002-6)

A mixture of 2-mercapto-5,6,8,9,10,10a-hexahydropyrimido[5,4-g]indolizin-4-ol (2 g, 9 mmol) and 2-chloroacetic acid (1.69 g, 17.9 mmol) in H₂O (20 mL) was refluxed for 12 h. LCMS showed the reaction was completed. The reaction was concentrated in vacuo and extracted with ethyl acetate (50 mL×3). The combined organic layers were washed with sat. aq. NaHCO₃ solution (20 mL) and brine (30 mL), dried over Na₂SO₄, and evaporated in vacuo to give 5,6,8,9,10,10a-hexahydropyrimido[5,4-g]indolizine-2,4-diol (1.2 g, 58.2%) as orange oil. LCMS (M+H⁺) m/z: Calcd: 208.10; Found: 208.2.

### 2,4-Dichloro-5,6,8,9,10,10a-hexahydropyrimido[5,4-g]indolizine (002-7)

A solution of 5,6,8,9,10,10a-hexahydropyrimido[5,4-g]indolizine-2,4-diol (1.1 g, 5.31 mmol) in POCl₃ (20 mL) was stirred at 110° C. for 2 hr. LCMS showed the reaction was completed. The reaction was concentrated in vacuo. The residue was purified by column chromatography (silica gel, petroleum ether, ethyl acetate) to give 2,4-dichloro-5,6,8,9,10,10a-hexahydropyrimido[5,4-g]indolizine (1.2 g, 83.3%) as light yellow solid. LCMS (M+H⁺) m/z: Calcd: 244.03; Found: 244.1.

### N-Benzyl-2-chloro-5,6,8,9,10,10a-hexahydropyrimido[5,4-g]indolizin-4-amine (002-8)

A solution of 2,4-dichloro-5,6,8,9,10,10a-hexahydropyrimido[5,4-g]indolizine (1.1 g, 4.5 mmol), phenylmethanamine (0.54 ml, 5.0 mmol) and TEA (1.26 ml, 9 mmol) in MeCN (50 mL) was stirred at 80° C. for 12 hr. The reaction was concentrated in vacuo. The residue was purified by column chromatography (silica gel, petroleum ether, ethyl acetate) to give N-benzyl-2-chloro-5,6,8,9,10,10a-hexahydropyrimido[5,4-g]indolizin-4-amine (0.55 g, 34.9%) as white solid.

### 1-[4-(Benzylamino)-5,6,8,9,10,10a-hexahydropyrimido[5,4-g]indolizin-2-yl]-2-methyl-indole-4-carbonitrile (002-9)

A mixture of N-benzyl-2-chloro-5,6,8,9,10,10a-hexahydropyrimido[5,4-g]indolizin-4-amine (180 mg, 0.57 mmol), 2-methyl-1H-indole-4-carbonitrile (98 mg, 0.63 mmol), Pd₂(dba)₃ (104 mg, 0.11 mmol), X-Phos (54 mg, 0.11 mmol), Cs₂CO₃ (372 mg, 1.14 mmol) in dioxane (20 mL) was refluxed for 2 hr under nitrogen. LCMS showed the reaction was completed. The reaction was concentrated in vacuo. The residue was purified by column chromatography (silica gel, petroleum ether, ethyl acetate) to give 1-[4-(benzylamino)-5,6,8,9,10,10a-hexahydropyrimido[5,4-g]indolizin-2-yl]-2-methyl-indole-4-carbonitrile (100 mg, 36.2%) as white solid. LCMS (M+H⁺) m/z: Calcd: 435.22; Found: 435.4.

### 1-[4-(Benzylamino)-5,6,8,9,10,10a-hexahydropyrimido[5,4-g]indolizin-2-yl]-2-methyl-indole-4-carboxamide (002)

A mixture of 1-[4-(benzylamino)-5,6,8,9,10,10a-hexahydropyrimido[5,4-g]indolizin-2-yl]-2-methyl-indole-4-carbonitrile (80 mg, 0.18 mmol), UHP (87 mg, 0.92 mmol), K₂CO₃ (25 mg, 0.18 mmol) in DMSO (5 mL) and H₂O (0.5 mL) was stirred at 15° C. for 12 hr. LCMS showed the reaction was completed. The reaction was diluted with ethyl acetate (20 mL) and washed by brine (20 mL×3). The organic layer was dried over Na₂SO₄ and concentrated in vacuo. The crude product was purified by prep-HPLC (FA) to give 1-[4-(benzylamino)-5,6,8,9,10,10a-hexahydropyrimido[5,4-g]indolizin-2-yl]-2-methyl-indole-4-carboxamide (40 mg, 48%) as orange solid. LCMS (M+H⁺) m/z: Calcd: 453.23; Found: 453.3. ¹HNMR (400 MHz, CDCl₃): δ 12.13 (s., 1H), 8.29 (t, J=5.8 Hz, 1H, NH), 7.86 (d, J=8.5 Hz, 1H, Ph), 7.47 (d, J=7.5 Hz, 1H, Ph), 7.35 (d, J=4.0 Hz, 3H, Ph), 7.29-7.21 (m, 1H, Ph), 6.95 (t, J=7.8 Hz, 1H, Ph), 6.88 (s, 1H, 3-H-indole), 6.63 (s., 4H, NH), 4.69 (d, J=5.0 Hz, 2H, ArCH₂), 4.64-4.55 (m, 1H, CH₂), 3.61 (s., 2H, CH₂), 3.49-3.31 (m, 2H, CH₂), 2.99-2.82 (m, 2H, CH₂), 2.29-2.16 (m, 1H, CH₂), 2.05 (s., 2H, CH₂).

### Example 03

### Synthesis of 1-(4-(benzylamino)-7,8,9,10,10a,11-hexahydro-5H-pyrimido[4,5-b]quinolizin-2-yl)-2-methyl-1H-indole-4-carboxamide (003)

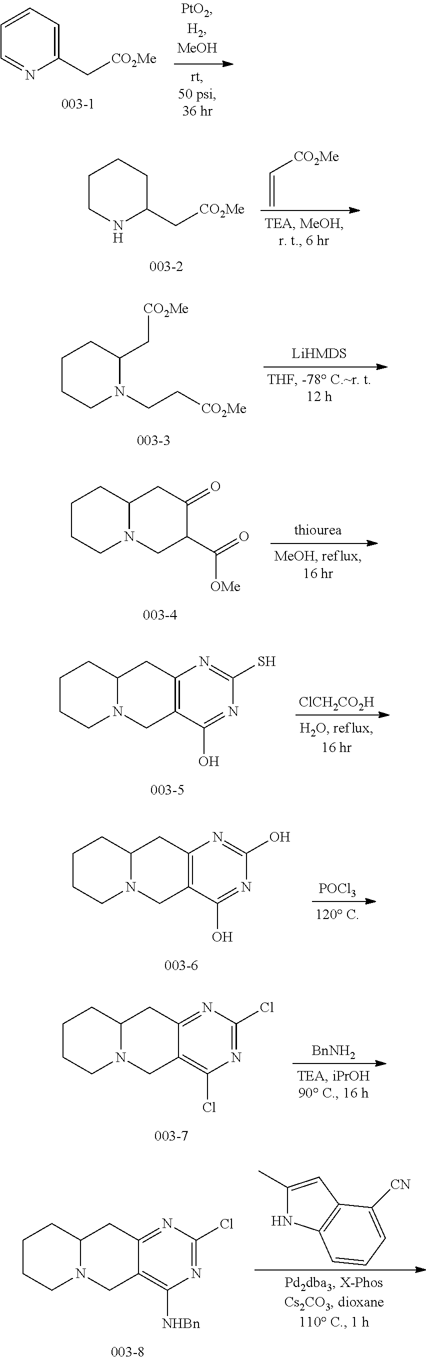

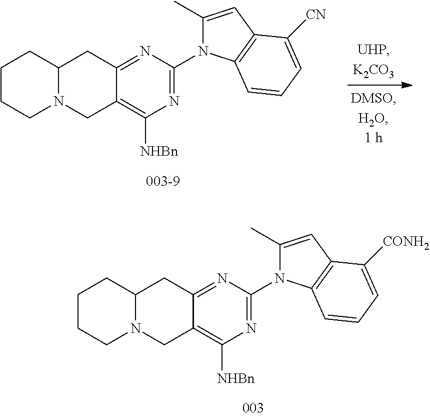

### Methyl 2-(2-piperidyl)acetate (003-2)

To a 25° C. solution of methyl 2-(2-pyridyl)acetate (10 g, 66.15 mmol) in MeOH (100 mL) was added PtO₂ (3 g, 13.2 mmol). The reaction was stirred for 48 hr under H₂ (50 psi). LCMS showed the reaction was completed. The catalyst was removed and the residue was concentrated to give methyl 2-(2-piperidyl)acetate (10 g, 96.2%) as yellow oil. LRMS (M+H⁺) m/z: Calcd: 157; Found: 157.1.

### Methyl 3-[2-(2-methoxy-2-oxo-ethyl)-1-piperidyl]propanoate (003-3)

To a 25° C. solution of methyl 2-(2-piperidyl)acetate (10 g, 63.6 mmol) and methyl prop-2-enoate (16.4 g, 190.8 mmol) in MeOH (100 mL) was added TEA (38.6 g, 381.7 mmol). The reaction was stirred for 3 hr. LCMS showed the reaction was completed. The reaction was diluted with ethyl acetate (20 mL) and extracted with DCM (20 mL×3). The combined organic layers were dried over Na₂SO₄ and concentrated in vacuo to give methyl 3-[2-(2-methoxy-2-oxo-ethyl)-1-piperidyl]propanoate (10.9 g, 70.2%) as yellow oil. LRMS (M+H⁺) m/z: Calcd: 244; Found: 244.1.

### Methyl 2-oxooctahydro-1H-quinolizine-3-carboxylate (003-4)

A solution of methyl3-[2-(2-methoxy-2-oxo-ethyl)-1-piperidyl]propanoate (10.9 g, 44.64 mmol) in THF (200 mL) was added LiHMDS (89.3 mL, 89.27 mmol) dropwise at −78° C. under nitrogen. The reaction was slowly warmed to 15° C., and stirred for 12 hr. LCMS showed the reaction was completed. The reaction was quenched by aqueous HCl (4 N, 20 mL). The mixture was extracted by DCM (200 mL×3). The combined organic layers were dried over Na₂SO₄, concentrated in vacuo to give methyl 2-oxooctahydro-1H-quinolizine-3-carboxylate (7.2 g, 76.4%) as white powder. LRMS (M+H⁺) m/z: Calcd: 212; Found: 212.2.

### 2-Mercapto-7,8,9,10,10a,11-hexahydro-5H-pyrimido[4,5-b]quinolizin-4-ol (003-5)

To a 25° C. solution of methyl 2-oxooctahydro-1H-quinolizine-3-carboxylate (6.8 g, 32.19 mmol) and thiourea (3.68 g, 48.28 mmol) in MeOH (50 mL) was added t-BuOK (7.22 g, 64.38 mmol). The reaction was stirred at 70° C. for 16 hr. LCMS showed the reaction was completed. The reaction was quenched by water (20 mL). The mixture was extracted by ethyl acetate (100 mL×3). The combined organic layers were dried over Na₂SO₄, concentrated in vacuo to give 2-mercapto-7,8,9,10,10a,11-hexahydro-5H-pyrimido[4,5-b]quinolizin-4-ol (4 g, 52.4%) as yellow powder. LRMS (M+H⁺) m/z: Calcd: 238; Found: 238.2.

### 7,8,9,10,10a,11-Hexahydro-5H-pyrimido[4,5-b]quinolizine-2,4-diol (003-6)

To a 25° C. solution of 2-mercapto-7,8,9,10,10a,11-hexahydro-5H-pyrimido[4,5-b]quinolizin-4-ol (4 g, 20 mmol) in H₂O (50 mL) was added 2-chloroacetic acid (4.78 g, 50.56 mmol). The reaction was stirred at 100° C. for 16 hr. LCMS showed the reaction was completed. To the reaction was quenched by NaHCO₃ (4 g) and its pH was adjusted to 7. The precipitated solids were collected and dried to give 7,8,9,10,10a,11-hexahydro-5H-pyrimido[4,5-b]quinolizine-2,4-diol (2.1 g, 56.3%) as white powder. LRMS (M+H⁺) m/z: Calcd: 222; Found: 222.2.

### 2,4-Dichloro-7,8,9,10,10a,11-hexahydro-5H-pyrimido[4,5-b]quinolizine (003-7)

To a 25° C. solution of 7,8,9,10,10a,11-hexahydro-5H-pyrimido[4,5-b]quinolizine-2,4-diol (1.9 g, 8.6 mmol) in POCl₃ (20 ml) was added DMAP (2.1 g, 17.2 mmol). The reaction was stirred at 100° C. for 16 hr. LCMS showed the reaction was completed. To the reaction was quenched by NaHCO₃ and its pH was adjusted to 7. The mixture was extracted by ethyl acetate (50 mL×3). The combined organic layers were dried over Na₂SO₄, concentrated in vacuo to give 2,4-dichloro-7,8,9,10,10a,11-hexahydro-5H-pyrimido[4,5-b]quinolizine (1.4 g, 63.2%) as white solid. LRMS (M+H⁺) m/z: Calcd: 258; Found: 258.1.

### N-Benzyl-2-chloro-7,8,9,10,10a,11-hexahydro-5H-pyrimido[4,5-b]quinolizin-4-amine (003-8)

To a 25° C. solution of 2,4-dichloro-7,8,9,10,10a,11-hexahydro-5H-pyrimido[4,5-b]quinolizine (150 mg, 0.58 mmol) and phenylmethanamine (63 mg, 0.58 mmol) in i-PrOH (10 ml) was added TEA (177 mg, 1.74 mmol). The reaction was stirred at 80° C. for 16 hr. LCMS showed the reaction was completed. The reaction was concentrated in vacuo and the residue was purified by prep-TLC (silica gel, petroleum ether, ethyl acetate) to give N-benzyl-2-chloro-7,8,9,10,10a,11-hexahydro-5H-pyrimido[4,5-b]quinolizin-4-amine (150 mg, 78.5%) as white powder. LRMS (M+H⁺) m/z: Calcd: 329; Found: 329.2.

### 1-[4-(Benzylamino)-7,8,9,10,10a,11-hexahydro-5H-pyrimido[4,5-b]quinolizin-2-yl]-2-methyl-indole-4-carbonitrile (003-9)

To a 25° C. solution of N-benzyl-2-chloro-7,8,9,10,10a,11-hexahydro-5H-pyrimido[4,5-b]quinolizin-4-amine (130 mg, 0.4 mmol) and 2-methyl-1H-indole-4-carbonitrile (62 mg, 0.4 mmol) in dioxane (5 mL) was added Pd₂(dba)₃ (36 mg, 0.04 mmol), X-phos (38 mg, 0.08 mmol) and Cs₂CO₃ (257 mg, 0.8 mmol). The reaction was stirred at 110° C. for 1 hr. LCMS showed the reaction was completed. The catalyst was removed and the residue was concentrated in vacuo to give crude product, and it was purified by prep-TLC (silica gel, petroleum ether, ethyl acetate) to give of 1-[4-(benzylamino)-7,8,9,10,10a,11-hexahydro-5H-pyrimido[4,5-b]quinolizin-2-yl]-2-methyl-indole-4-carbonitrile (120 mg, 65.1%) as yellow powder. LRMS (M+H⁺) m/z: Calcd: 449; Found: 449.3.

### 1-[4-(Benzylamino)-7,89,10,10a,11-hexahydro-5H-pyrimido[4,5-b]quinolizin-2-yl]-2-methyl-indole-4-carboxamide (003)

To a 25° C. solution of 1-[4-(benzylamino)-7,8,9,10,10a,11-hexahydro-5H-pyrimido[4,5-b]quinolizin-2-yl]-2-methyl-indole-4-carbonitrile (100 mg, 0.22 mmol) in DMSO (2 mL) and H₂O (0.2 mL) were added UHP (105 mg, 1.11 mmol) and K₂CO₃ (31 mg, 0.22 mmol). The reaction was stirred for 2 hr. LCMS showed the reaction was completed. The reaction was diluted with water (5 mL) and extracted with ethyl acetate (5 mL×3). The combined organic layers were washed with brine (5 mL) and dried over Na₂SO₄ and concentrated in vacuo. The residue was purified by prep-HPLC to give 1-[4-(benzylamino)-7,8,9,10,10a,11-hexahydro-5H-pyrimido[4,5-b]quinolizin-2-yl]-2-methyl-indole-4-carboxamide (7.8 mg, 7.5%) as white powder. LRMS (M+H⁺) nm/z: Calcd: 467; Found: 467.3. ¹H NMR (400 MHz, DMSO-d₆): δ 1.19-1.39 (m, 2H, CH₂) 1.56 (d, J=11.47 Hz, 1H, CH) 1.65-1.79 (m, 2H, CH₂) 1.85 (d, J=8.82 Hz, 1H, CH₂) 2.19 (t, J=10.81 Hz, 1H, CH₂) 2.33 (d, J=6.62 Hz, 1H, CH₂) 2.46 (s, 3H, CH₃) 2.55-2.59 (m, 1H, CH₂) 2.66-2.73 (m, 1H, CH₂) 3.05 (d, J=15.44 Hz, 2H, CH₂) 3.81 (d, J=15.44 Hz, 2H, CH₂) 4.64 (d, J=5.73 Hz, 2H, CH₂) 6.83 (s, 1H, Ar) 6.92 (t, J=7.72 Hz, 1H, Ar) 7.14-7.27 (m, 2H, NH₂) 7.27-7.37 (m, 3H, Ar) 7.44 (d, J=7.50 Hz, 1H, Ar) 7.62-7.76 (m, 2H, NH₂) 7.84 (d, J=8.38 Hz, 1H, Ar) 8.16 (s, 2H, Ar).

### Example 04

### Synthesis of 1-(4-(benzylamino)-6,8,9,10,11,11a-hexahydro-5H-pyrimido[4,5-a]quinolizin-2-yl)-2-methyl-1H-indole-4-carboxamide (004)

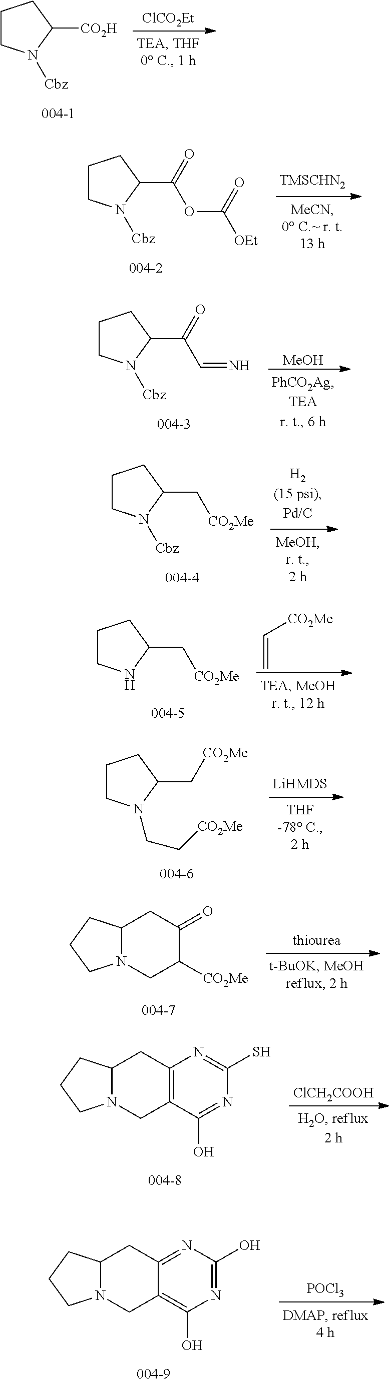

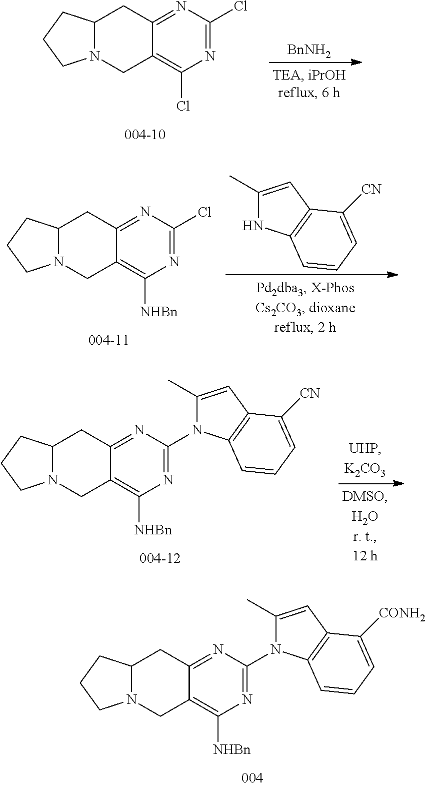

### 1-((Benzyloxy)carbonyl)pyrrolidine-2-carboxylic (Ethyl Carbonic) Anhydride (004-2)

To a 0° C. solution of 1-benzyloxycarbonylpyrrolidine-2-carboxylic acid (1 g, 4.01 mmol) in THF (20 mL) were added TEA (0.6 ml, 4.01 mmol) and ethyl carbonochloridate (0.4 ml, 4.01 mmol) dropwise. The reaction was stirred for 1 h. TLC showed the reaction was completed. The reaction was quenched by sat. aq. NH₄Cl (10 mL) and extracted with ethyl acetate (20 mL×3). The combined organic layers were dried over Na₂SO₄, concentrated in vacuo. The residue was purified by column chromatography (silica gel, petroleum ether, ethyl acetate) to give 1-((benzyloxy)carbonyl)pyrrolidine-2-carboxylic (ethyl carbonic) anhydride (0.6 g, 46.5%) as oil. LRMS (M+H⁺) m/z: Calcd: 322.12; Found: 322.

### Benzyl 2-(2-iminoacetyl)pyrrolidine-1-carboxylate (004-3)

To a 0° C. solution of 1-((benzyloxy)carbonyl)pyrrolidine-2-carboxylic (ethyl carbonic) anhydride (70%, 30 g, 78.44 mmol), TEA (22 mL, 156.89 mmol) in MeCN (100 mL) and THF (300 mL) was added diazomethyl(trimethyl)silane (2M, 78.44 ml). The reaction was stirred at 0° C. for 1 h. Then warmed to 25° C., and stirred for additional 12 hr. TLC showed the reaction was completed. The reaction was quenched by AcOH (10 mL). The mixture was concentrated in vacuo. The residue was purified by column chromatography (silica gel, petroleum ether, ethyl acetate) to give benzyl 2-(2-iminoacetyl)pyrrolidine-1-carboxylate (17 g, 74.9%) as oil. LRMS (M+H⁺) m/z: Calcd: 261.12; Found: 261.

### Benzyl 2-(2-methoxy-2-oxo-ethyl)pyrrolidine-1-carboxylate (004-4)

To a 25° C. solution of benzyl 2-(2-iminoacetyl)pyrrolidine-1-carboxylate (17 g, 65.31 mmol) in MeOH (400 mL) was added a solution of PhCO₂Ag (3 g, 13.06 mmol) in TEA (36.4 mL, 261.25 mmol). The mixture was stirred for 6 h. TLC showed the reaction was completed. The reaction was filtered. The residue was concentrated in vacuo and purified by column chromatography (silica gel, petroleum ether, ethyl acetate) to give of benzyl 2-(2-methoxy-2-oxo-ethyl)pyrrolidine-1-carboxylate (15 g, 74.5%) as oil. LRMS (M+H⁺) m/z: Calcd: 278.13; Found: 278.

### Methyl 2-pyrrolidin-2-ylacetate (004-5)

A 25° C. mixture of benzyl 2-(2-methoxy-2-oxo-ethyl)pyrrolidine-1-carboxylate (15 g, 54.09 mmol) and Pd/C (10%, 10 g, 8.44 mmol) in MeOH (200 mL) was stirred under H₂ (15 psi) for 2 hr. TLC showed the reaction was completed. The reaction was filtered. The residue was concentrated in vacuo and purified by column chromatography (silica gel, petroleum ether, ethyl acetate) to give methyl 2-pyrrolidin-2-ylacetate (9 g, 87.2%) as oil. LRMS (M+H⁺) m/z: Calcd: 144.09; Found: 144.

### Methyl 3-[2-(2-methoxy-2-oxo-ethyl)pyrrolidin-1-yl]propanoate (004-6)

A 25° C. solution of methyl 2-pyrrolidin-2-ylacetate (7.5 g, 52.38 mmol), TEA (21.9 mL, 157.14 mmol) in MeOH (200 mL) was stirred for 30 min. Then methyl prop-2-enoate (9.4 mL, 104.76 mmol) was added dropwise. The reaction was stirred for 12 h. TLC showed the reaction was completed. The reaction was concentrated in vacuo and purified by column chromatography (silica gel, petroleum ether, ethyl acetate) to give methyl 3-[2-(2-methoxy-2-oxo-ethyl)pyrrolidin-1-yl]propanoate (9 g, 67.4%) as oil. LRMS (M+H⁺) m/z: Calcd: 230.13; Found: 230.

### Methyl 7-oxooctahydroindolizine-6-carboxylate (004-7)

A solution of methyl 3-[2-(2-methoxy-2-oxo-ethyl)pyrrolidin-1-yl]propanoate (9.5 g, 41.44 mmol) in THF (400 mL) was added LiHMDS (1M, 82.87 ml) dropwise at −78° C. under nitrogen. The reaction was stirred at the same temperature for 2 hr. TLC showed the reaction was completed. The reaction was quenched by H₂O (50 mL) and extracted with DCM (100 mL×3). The combined organic layers were dried over Na₂SO₄, concentrated in vacuo to give methyl 7-oxo-2,3,5,6,8,8a-hexahydro-1H-indolizine-6-carboxylate (3 g, 33%) as oil. LRMS (M+H⁺) m/z: Calcd: 198.11; Found: 198.

### 2-Mercapto-5,7,8,9,9a,10-hexahydropyrimido[5,4-f]indolizin-4-ol (004-8)

A solution of methyl 7-oxooctahydroindolizine-6-carboxylate (4.0 g, 20.28 mmol), thiourea (3.1 g, 40.56 mmol), t-BuOK (4.6 g, 40.56 mmol) in MeOH (100 mL) was refluxed for 2 h. LCMS showed the reaction was completed. The reaction was concentrated in vacuo and then H₂O (100 mL) was added. The mixture was adjusted pH=7 by HCl (1N). The mixture was extracted with DCM (100 mL×3). The combined organic layers were dried over Na₂SO₄, concentrated in vacuo to give 2-mercapto-5,7,8,9,9a,10-hexahydropyrimido[5,4-f]indolizin-4-ol (1.8 g, 35.8%) as yellow solid. LCMS (M+H⁺) m/z: Calcd: 224.08; Found: 224.0.

### 5,7,8,9,9a,10-Hexahydropyrimido[5,4-f]indolizine-2,4-diol (004-9)

A mixture of 2-mercapto-5,7,8,9,9a,10-hexahydropyrimido[5,4-f]indolizin-4-ol (2.0 g, 8.96 mmol) and 2-chloroacetic acid (1.7 g, 17.91 mmol) in H₂O (50 mL) was refluxed for 2 hr. LCMS showed the reaction was completed. The reaction was cooled down to room temperature and aqueous NaHCO₃ solution was added to adjust pH=7, the precipitated solids were collected and dried to give 5,7,8,9,9a,10-hexahydropyrimido[5,4-f]indolizine-2,4-diol (0.9 g, 43.6%) as white solid. LCMS (M+H⁺) m/z: Calcd: 208.10; Found: 208.2.

### 2,4-Dichloro-5,7,8,9,9a,10-hexahydropyrimido[5,4-f]indolizine (004-10)

A mixture of 5,7,8,9,9a,10-hexahydropyrimido[5,4-f]indolizine-2,4-diol (550 mg, 2.65 mmol), DMAP (324.24 mg, 2.65 mmol) in POCl₃ (20 mL) was refluxed for 4 hr. LCMS showed the reaction was completed. The reaction was concentrated in vacuo. The residue was dissolved in DCM (50 mL), washed with sat.NaHCO₃ (100 mL×2), dried over Na₂SO₄, concentrated in vacuo and purified by column chromatography (silica gel, petroleum ether, ethyl acetate) to give 2,4-dichloro-5,7,8,9,9a,10-hexahydropyrimido[5,4-f]indolizine (300 mg, 41.7%) as light yellow solid. LCMS (M+H⁺) m/z: Calcd: 244.03; Found: 244.1.

### N-Benzyl-2-chloro-5,7,8,9,9a,10-hexahydropyrimido[5,4-f]indolizin-4-amine (004-11)

A solution of 2,4-dichloro-5,7,8,9,9a,10-hexahydropyrimido[5,4-f]indolizine (900 mg, 3.69 mmol), phenylmethanamine (0.4 ml, 3.69 mmol), TEA (1.03 ml, 7.37 mmol) in i-PrOH (20 ml) was refluxed for 6 hr. TLC showed the reaction was completed. The reaction was concentrated in vacuo and the residue was purified by column chromatography (silica gel, petroleum ether, ethyl acetate) to give N-benzyl-2-chloro-5,7,8,9,9a,10-hexahydropyrimido[5,4-f]indolizin-4-amine (500 mg, 38.8%) as white solid. LCMS (M+H⁺) m/z: Calcd: 315.13; Found: 315.2.

### 1-[4-(Benzylamino)-5,7,8,9,9a,10-hexahydropyrimido[5,4-f]indolizin-2-yl]-2-methyl-indole-4-carboxamide (004-12)

A mixture of N-benzyl-2-chloro-5,7,8,9,9a,10-hexahydropyrimido[5,4-f]indolizin-4-amine (150 mg, 0.48 mmol), 2-methyl-1H-indole-4-carbonitrile (82 mg, 0.52 mmol), Pd₂(dba)₃ (87 mg, 0.1 mmol), X-Phos (45 mg, 0.1 mmol), Cs₂CO₃ (310 mg, 0.95 mmol) in dioxane (20 mL) was refluxed for 2 hr under nitrogen. LCMS showed the reaction was completed. The solid was removed, and the residue was concentrated in vacuo and purified by column chromatography (silica gel, petroleum ether, ethyl acetate) to give 1-[4-(benzylamino)-5,7,8,9,9a,10-hexahydropyrimido[5,4-f]indolizin-2-yl]-2-methyl-indole-4-carboxamide (100 mg, 41.7%) as white solid. LCMS (M+H⁺) m/z: Calcd: 435.22; Found: 435.3.

### 1-[4-(Benzylamino)-5,7,8,9,9a,10-hexahydropyrimido[5,4-f]indolizin-2-yl]-2-methyl-indole-4-carboxamide (004)

A 10° C. solution of 1-[4-(benzylamino)-5,7,8,9,9a,10-hexahydropyrimido[5,4-f]indolizin-2-yl]-2-methyl-indole-4-carbonitrile (90 mg, 207 μmol), UHP (97 mg, 1035 μmol), K₂CO₃ (29 mg, 207 μmol) in DMSO (5 mL) and H₂O (0.5 mL) was stirred for 12 hr. TLC showed the reaction was completed. The reaction was diluted with ethyl acetate (20 mL), washed with brine (20 mL×3). The organic layer was dried over Na₂SO₄, concentrated in vacuo. The residue was purified by column chromatography (silica gel, petroleum ether, ethyl acetate) to give 1-[4-(benzylamino)-5,7,8,9,9a,10-hexahydropyrimido[5,4-f]indolizin-2-yl]-2-methyl-indole-4-carboxamide (30 mg, 35.3%) as white solid. LCMS (M+H⁺) m/z: Calcd: 453.23; Found: 453.2. ¹HNMR (400 MHz, CDCl₃): δ 8.17 (d, J=8.5 Hz; 1H, Ph), 7.52 (d, J=7.5 Hz, 1H, Ph), 7.41-7.30 (m, 5H, Ar), 7.10 (t J=7.8 Hz, 1H, Ph), 6.83 (s, 1H, 3-H-indole), 4.85-4.75 (m, 3H, ArCH₂), 3.94 (d, J=14.1 Hz, 1H, CH), 3.35 (t, J=8.3 Hz, 1H, CH₂), 3.16 (d, J=14.1 Hz, 1H, CH₂), 3.06 (d, J=15.1 Hz, 1H, CH₂), 2.77 (m, J=10.5, 17.1 Hz, 1H, CH₂), 2.64 (s, 3H, Me), 2.51 (d, J=8.5 Hz, 1H, CH₂), 2.37 (m, J=8.5 Hz, 1H, CH₂), 2.17 (d, J=5.0 Hz, 1H, CH₂), 2.05-1.96 (m, 1H, CH₂), 1.95-1.85 (m, 1H, CH₂), 1.72-1.58 (m, 2H, CH₂)

### Biological Protocols

The in vitro and in vivo biological assays to determine the anti-cancer properties of the compounds of the invention are summarized above. The details of these protocols show how the assays are carried out.

### P97 Biochemical Assay Protocol

The p97 assay is an initial screening assay used to determine inhibitory activity of the compounds of the invention against the p97 complex. As discussed above, inhibition of activity of the p97 proteosome complex can enable apoptosis and cause elimination of neoplastic cells (cancer cells). The method follows that of Christianson in Nat. Cell Biol., (2011) 14:93.

The Reagents Used for the p97 Assay Include:

Assay Buffer is a mixture of 50 mM TRIS pH 7.5, 20 mM MgCl₂, 0.02% TX-100, 1 mM DTT and 0.2% (v/v) Glycerol. The well plate is Platetype: Corning 3674, 384 w plate. The identification kit is an ADP glo kit (Promega): stop buffer, detection reagent.

The Assay Protocol is Conducted as Follows:

Serial dilute compound in DMSO in a 1:3.33-fold 10 point serial dilution.

in each well of 384 w plate add the following reagents:

0.5 μL compound serial diluted in DMSO (Final Conc. 10%)

2 μL ATP (Final Conc.=20 uM, diluted in assay buffer)

2.5 μL p97 (Final Conc.=20 nM, diluted in assay buffer)

Incubate at 37 deg C. for 15 min.

Add 5μ of stop buffer, incubate at RT for 40 min.

Add 10 μL of detection reagent, incubate at RT for 30 min.

Read luminescence on Envision plate reader.

Upon obtaining the data from the luminescence reading, the data may be analyzed as follows:

Normalize luminescence data using no enzyme (full inhibition) and no compound (no inhibition) controls. Plot normalized luminescence data against log-transformed concentration values and fit to a sigmoidal curve to determine IC50 values (done in Collaborative Drug Discovery software).

### Caco-2 Permeability Assay

This assay is designed as a model to indicate the permeability of a compound of this invention through the gut-blood barrier. The result will yield indications of whether or not the compound may be efficiently absorbed into the blood stream of a patient. Efficient, effective absorption of an orally administered drug determines in part its bioavailability. For the compounds of the invention, this assay is a model to evaluate the bioavailability of the compounds as a result of their ability to pass through biological barriers to entry into the physiological system of the patient.

The experimental goal of the Caco-2 assay is to measure directional Caco-2 permeability of test compounds in cultured Caco-2 monolayer.

The test compounds are the compounds of the invention.

### Set-Up

Instruments
Tissue culture CO₂ incubator with humidity control
Liquid handler
Orbital shaker
EVOM Epithelial Volt-ohmmeter fitted with planar electrodes (World Precision Instruments, Sarasota, Fla.) required for measuring transepithelial electrical resistance (TEER)
Bench top centrifuge with 96-well plate adaptor
Caco-2 cells (Human colorectal adenocarcinoma, ATCC #37-HTB, passage 30-45)
Cells seeded onto PET membranes (1 μm pore size, 0.31 cm² surface area) inside Falcon HTS multiwell Insert system using 24-well plates (Becton Dickinson plates, Part #351181, Fisher Scientific, Inc.) at a density of 23,000 cells/well. Cells grown 20-23 days with medium changed every 2-3 days

### Reagents

Ringers buffer solution (pH 7.4 at 25° C.)
Ringers buffer with 1% Methanol
Blk solution: Ringers buffer: Methanol=2:1 (v/v); 100% Methanol including internal standard (IS); 10 mM stock dosing solution in DMSO; 100 μM dosing solution in buffer.

### Protocol Summary

Caco-2 permeability: 20-23 day/Passage 30-45
24-well format transwell: 0.31 cm2 surface area
Donor conc: 100 μM including 1% DMSO
A: 300 μL pH 7.4/B: 1200 μL pH 7.4 Ringers buffer
Directionality: A B and B A (N=4)
Donor side sampling: 20 μL at beginning and end (90 min)
Receiver side sampling: 100 μL at 30, 50, 70, and 90 min
Incubation at 50 oscillations per minute, 37° C., 5% CO₂, 95% humidity
Analysis: LC-UV, LC-MS, or LSC
   Output: Peff (cm/sec)=(dX/dt)/(A*Co*60), dX/dt: transported amount (nmole) versus time (minute) profile in the receiver chamber; A: surface area (cm²); and Co: initial donor concentration (μM)
Positive control: Atenolol and propranolol
Membrane integrity: TEER>200 Ocm²
   Amount required: Approximately 1 mg or 100 μL of 10 mM test compound in DMSO
   Instruments: CO₂ incubator with humidity control, liquid handler, epithelial volt-ohmmeter for TEER, Caco-2 cells (ATCC #37-HTB), and 24-well insert plates (PET membranes, 1 μm pore size, 0.31 cm² plates, Part #351181) surface area, Becton Dickinson;
Throughput: 6 compounds/2 Caco-2 plates/1 FTE/day

TABLE 24
Preparation of Ringers with Glucose (Isotonic = 290 mOsm/kg), pH 7.4

| | | | Mass | Mass | Mass |
|---|---|---|---|---|---|
| | Molecular | | (g) for | (g) for | (g) for |
| Chemical | Wt | Concentration | 1 L | 2 L | 4 L |
| Ca SO4 2H2O | 172.2 | 1.25 mM | 0.2152 | 0.4305 | 0.861 |
| MgSO4 7H2O | 246.5 | 1.1 mM | 0.2712 | 0.5423 | 1.0846 |
| KCl | 74.55 | 5 mM | 0.3728 | 0.7455 | 1.491 |
| Na2HPO4 | 142 | 1.15 mM | 0.1633 | 0.3266 | 0.6532 |
| NaH2PO4 H2O | 138 | 0.3 mM | 0.0414 | 0.0828 | 0.1656 |
| NaHCO3 | 84.01 | 25 mM | 2.1 | 4.2 | 8.401 |
| Glucose | 180.2 | 25 mM | 4.505 | 9.01 | 18.02 |
| (C6H12O6) | | | | | |
| NaCl | 58.44 | 110 mM | 6.428 | 12.86 | 25.71 |

### Preparation of 4 L Solution

1. To 3.5 L distilled water, add Calcium Sulfate and Magnesium Sulfate.
Note: Add Calcium Sulfate and Magnesium Sulfate first due to low solubility and add the remaining ingredients in the order listed in Table 1.

2. Adjust the final volume of the solution to 4 L with distilled water, with continuous stirring.

3. Adjust final solution to a pH of 7.4 using 1N HCl or 1N NaOH.

4. Make the buffer iso-osmotic using NaCl. Measure tonicity of the solution using a tonometer. Given that an isotonic solution is equivalent to 0.9% NaCl (290 mOsm/L),
Y={((290−x)/290}×9 mg×4000 mL, where y=NaCl required (in mg) to make the solution isotonic and x=observed tonicity of solution (reported as mOsm/L).

Preparation of Dosing Solution in 15 ml PP Tube

1. 100 μM dosing solution in RG: 140 μL 10 mM stock+(14 mL-140 μL) RG

Preparation of Calibration in 96 Shallow Well

1. Prepare 10 μM standard: 100 μL of 100 μM dosing solution+0.9 mL Ringers with 1% Methanol.

2. Prepare analytical standard solutions 10, 5, 2, 1, 0.5, 0.2, 0.1, 0.05, 0.02, 0.01, and 0 μM. (See Table 26)

TABLE 25
Preparation of analytical calibration in 96 shallow well

| | 1 | 2 | 3 | 4 | 5 | 6 | 7 | 8 | 9 | 10 | 11 | 12 |
|---|---|---|---|---|---|---|---|---|---|---|---|---|
| | 0 | 20 μL of | 20 μL of | 20 μL of | 20 μL of | 20 μL of | 20 μL of | 20 μL of | 40 μL of | 100 μL of | 200 μL of | Source |
| | | 0.1 μM | 0.2 μM | 0.5 μM | 1 μM | 2 μM | 5 μM | 10 μM | 10 μM | 10 μM | 10 μM | solution |
| | 180 μL | 180 μL | 180 μL | 180 μL | 180 μL | 180 μL | 180 μL | 180 μL | 160 μL | 100 μL | 0 | 1% MeOH |
| | | | | | | | | | | | | in buffer |
| Comp 1 | Blk | 0.01 μM | 0.02 μM | 0.05 μM | 0.1 μM | 0.2 μM | 0.5 μM | 1 μM | 2 μM | 5 μM | 10 μM | |
| Comp 2 | | | | | | | | | | | | |
| Comp 3 | | | | | | | | | | | | |

### Transport Studies

### Dosing and Sampling

1. Equilibrate both sides of the monolayers for 10 minutes with prewarmed (37° C.) drug-free Ringers buffer (300 μL apical side, 1,200 μL basolateral side) supplemented with glucose (25 mM).

2. Measure TEER under 37° C. water bath conditions.

Note: The TEER value serves as a quality control check for monolayer integrity. At 21 days post-seeding, each Caco-2 cell monolayer should have a TEER value of greater than or equal to 2000×cm² and those not meeting this criteria are not suitable for permeability evaluations.

3. When studying A to B transport: Fill basolateral side with 1,200 μL of Ringers buffer. Initiate transport experiments by transferring test drug dosing solution (320 μL) to apical side.

4. When studying B to A transport: Fill apical side with 300 μL of Ringers buffer. Initiate transport experiments by transferring test drug dosing solution (1,220 μL) to basolateral side. Transport studies for each direction (A to B, B to A) are performed in quadruplicate for each test drug.

5. Start timer after dosing last donor well.

6. Remove 20 μL aliquots from the donor wells at 0 minutes (D_{O}) and transfer these aliquots to the donor site of the 96-well plate containing 180 μL buffer with 1% Methanol. This step effectively dilutes the D₀ ten times.

7. Initiate transport studies by placing plate on orbital shaker maintained inside a prewarmed (37° C.) and humidified (5% CO₂) incubator. Studies are performed under stirring conditions at 50 oscillations per minute.

8. Remove 100 μL aliquots from the receiver side of the monolayer at 30, 50, 70, and 90 minutes postdosing and transfer these aliquots to the corresponding 96-well sample plate (See Table 26). Replace with an equivalent volume of prewarmed buffer.

9. Remove 20 μL aliquots from the donor side of the monolayer at 90 minutes postdosing (D_{f}) and transfer these aliquots to a donor site of a 96-well plate containing 180 μL Ringers buffer with 1% Methanol. This step effectively dilutes the D_{f} ten times.

10. Replace both sides of monolayer with fresh, drug-free, prewarmed Ringers buffer (300 μL apical side, 1,200 μL basolateral side) and equilibrate for 10 minutes.

11. Measure TEER under 37° C. water bath conditions.

### Sample Handling

The following steps refer to 96-well analytical plate for Caco-2, Table 26.

1. Transfer 20 μL of diluted D₀ and D_{f} to corresponding 96-well sample plate with each well containing 80 μL buffer with 1% Methanol. This step effectively dilutes the samples five times further. Therefore, donor samples are diluted 50 times from their initial concentration.

2. Transfer 100 μL of analytical calibration (from 0 to 10 μM) to the sample plate row 1.

3. Add 50 μL Methanol including IS to all sample wells and mix (standards, samples, and D₀ and D_{f}).

4. Transfer 150 μL of Blk solution to the analytical plate row 2.

5. Seal the analytical plate with adhesive sealing film and store samples with label at −80° C. for LC-UV or LC-MS analysis.

6. Analyze 20 μL aliquots of the individual permeability samples and the standards using a suitable analytical instrument.

7. Peff=(dX/dt)/(A×C₀×60), where P_{eff} is the effective permeability in cm/sec, X=mass transported, A is the surface area (cm)² available for transport, C₀ is the initial donor drug concentration (μM), and dX/dt is the slope of the best fit line through the transported amount (nmole) versus time (min) profile in the receiver chamber.

TABLE 26
Analytical Plate for Caco-2 (96-well plate)

| 0 | 0.01 μM | 0.02 μM | 0.05 μM | 0.1 μM | 0.2 μM | 0.5 μM | 1 μM | 2 μM | 5 μM | 10 μM | |
|---|---|---|---|---|---|---|---|---|---|---|---|
| Blk | Blk | Blk | Blk | A to B | | | B to A | Blk | Blk | Blk | Blk |
| 30-Jan | Feb-30 | 30-Mar | 30-Apr | | | | | 30-May | 30-Jun | 30-Jul | 30-Aug |
| Jan-50 | Feb-50 | Mar-50 | Apr-50 | | | | | May-50 | Jun-50 | Jul-50 | Aug-50 |
| Jan-70 | Feb-70 | Mar-70 | Apr-70 | | | | | May-70 | Jun-70 | Jul-70 | Aug-70 |
| Jan-90 | Feb-90 | Mar-90 | Apr-90 | | | | | May-90 | Jun-90 | Jul-90 | Aug-90 |
| 1-D₀ | 2-D₀ | 3-D₀ | 4-D₀ | | | | | 5-D₀ | 6-D₀ | 7-D₀ | 8-D₀ |
| 1-Df | 2-Df | 3-Df | 4-Df | | | | | 5-Df | 6-Df | 7-Df | 8-Df |

### Positive Control Data

Mean data in Table 27 represent the mean value from 12 separate inter-day experiments.

TABLE 27
P_{eff} (x E−6 cm/sec) in
pH 7.4 Caco-2

| | A B | B A |
|---|---|---|
| Atenolol | | |
| Mean | 1.08 | 2.29 |
| Range | 0.69-1.80 | 1.69-2.68 |
| Propranolol | | |
| Mean | 28.53 | 20.91 |
| Range | 18.50-36.80 | 16.30-31.40 |

### Mouse Liver Microsome Assay

The liver microsome assay is a model for studying the metabolic stability of the compounds of the invention. Metabolic stability is another aspect determining bioavailability. The facility of a compound to be bioabsorbed into the blood stream as shown by the Caco-2 model indicates the degree to which an oral dose of the compound will reach the blood stream. The body efficiently metabolizes substances to rid them from the body and/or to utilize them as nutrients. This aspect of bioavailability can be determined by such model studies as liver microsomal metabolism. Whether by oxidation, conjugation or any other biological pathway, metabolism of a drug determines at least in part the lifetime of the drug in the body.

The mouse liver microsome assay is a model designed to establish drug half-life in vivo. The liver enzymes are responsible to conversion of substances to materials that can be readily excreted by the body. Other routes for such metabolism include kidney metabolism, cellular metabolism and the like.

In this protocol, the compound is combined with a liver microsomal preparation (protein) and NADPH. The mixture is incubated and the rate of disappearance of the compound from the test solution is measured. Measurement is made by screening for the compound concentration at specified times using liquid chromatography in combination with mass spectroscopy.

Concentrations of Reactants Ready for Formulation as the Test Solution:

Protein: 1.0 mg/ml

Compound: 1 um

Organic solvent: 0.4% DMSO

Medium: 0.1 M Potassium Phosphate (KB)

1 mM NADPH (sigma N1630, FW 833.3, make freshly)

Prepare test article (TA, i.e., a compound of the invention) by dissolving solid TA in DMSO to make a 0.25 mM solution

Amounts of Reactant Solutions to be Combined to Form the Test Solution:

| | |
|---|---|
| 423 ul | KB (potassium phosphate) |
| +25 ul | MLM (20 mg/ml) (mouse liver microsomal preparation) |
| 448 ul | |
| +2 ul | Test compound (a triazine compound at 0.25 mM DMSO) |
| +50 ul | NADPH stock (10 mM, 10 x) |
| 500 ul | |

Test Protocol for Conducting the Assay

1. Add 423 ul KB to an 8-strip deep well tubes

2. Add 25 ul of MLM for condition 1

3. Place on ice, add 2 ul cmpds (250× stock in DMSO, stock at 0.25 mM)

4. Preincubate the reaction mixture at 37 C for 3 to 5 minutes (shaking at 150 rpm)

5. Initiate reaction by adding 50 ul NADPH for condition 1

6. Add 50 ul KB for condition 2

7. An aliquot of samples of 100 ul were collected at 0, 15, 30, and 60 min time point, and 200 ul of acetonitrile mixture containing IS was added to quench the reaction.

8. Centrifuge for 10 min at 4000 rpm

9. The supernatant were injected for liquid chromatographic tandem mass spectrometry (LC-MS/MS) analysis

Procedure of Protein Binding Using 96-Well Equilibrium Dialyzer

Non-specific protein binding is another facet affecting bioavailability and effectiveness of a drug. To assay a compound for non-specific binding, the compound is combined with human blood plasma and the solution dialyzed against a membrane constructed to prevent passage of larger molecules such as human plasma proteins but allow passage of small molecules such as the compounds of the invention. Typically, such membranes allow passage of such compounds irrespective of their salt or neutral form. The dialysate (solution passing through the membrane) is examined by liquid chromatography mass spectrometric techniques to determine the identity and concentration of the compound present. The concentration of compound in the dialysate compared with the concentration of compound combined with blood plasma indicates whether or not non-specific protein binding has occurred.

Equipment and Reagent:

96-Well Equilibrium Dialyzer (made by: Harvard Apparatus)

Plate Rotator with DIALYZER plates secured in clamp fixture

Buffer: DPBS (gibco, 1×)

Compound Concentration: 1 μM (˜0.5 in μg/mL) in Human Plasma

Procedure:

1. Seal the empty Sample Side well on the colored side with cap strips.

2. Invert the plate and carefully pipet a volume of buffer, 200 μL equal to the sample volume into the wells on the Buffer Side (clear frame) without touching the membranes by allowing the liquid to flow along the inner side wall of each well.

3. Gently seal the filled buffer wells with cap strips.

4. Invert the plate and carefully remove the cap strips from the sample side wells. Pipet desired samples, without touching the membranes.

5. Reseal the sample wells with the cap strips.

6. Slide the assembled DIALYZER Plate into a Plate Rotator and hand tighten the snobs. Turn on and allow rotating until equilibrium has been reached (24 hours at 37 C), remove the DIALYZER Plate from the Rotator.

7. After equilibrium has been reached, remove the DIALYZER Plate from the rotator.

8. Carefully remove the cap strips from the Buffer Side of the Plated (clear frame) and slowly pipet out the analysis samples from the wells taking care not to touch or puncture the membranes.

Samples will include control at 4 C and stability at 37 C samples in PBS and plasma.

MS Analysis:

Prepare standard range 5, 10, 50, 100, 500 and 1000 ng/mL in Plasma

Pipet 10 μL each of standard and sample into 40 μL of blank buffer/blank plasma them (ratio: 1 plasma/4 DPBS), mix them.

Add 200 μL of Is (internal standard) in ACN, mix well.

Centrifuge the samples and transfer supernatant solution for LC/MS analysis.

### The Cell Assay Protocol

The cellular assay provides information about the anti-neoplastic activity of the compounds of the invention. The compounds are tested against cultured cancer cells to determine whether or not the compounds of the invention are capable of intersecting with cancer cells to minimize or eliminate such cells. The assay involves establishing colonies of such cells and then treating them with the test compound under specified conditions and analysis regima to determine results.

Day 1, Cell Plating to Establish Colonies of Cancer Cells

Cell Plating:

Seed cells ˜16 hrs prior to compound treatment

Plate 25 μL of A549 cells in every well of 384-well plate using multidrop.
Two (2) black plates for IF at 2500 cells/well
Let plate sit at room temp for 10-15 minutes prior to putting in incubator to allow cells to stick in middle of plate.
One (1) white plate for viability at 500 cells/well.

Day 2 Treatment of Cultured Cells with Test Compounds

Treat Cells:
Serial dilute compounds with a 10 point 2-fold serial dilution in DMSO to make 250× stock compound solution
Dilute compounds 1:125 in cell culture media to make a 2× solution Add 25 μL of dilution compounds to cell plates in well duplicates

Put cells back in incubator (6 hr incubation for black plates, 72 hr incubation for white plates).

Fix/Stain Black Plates:

Incubate cells in black plates with compound at 37 deg C. for 6 hrs.

add 15 μL of 16% Paraformaldehyde (PFA) directly into media of each well,
incubate at room temp for 5 min, flick plate and wash in 50 □L of PBS

block in 50 μL of Blocking Buffer for 30 minutes (can go up to several hours)
Blocking buffer: 1×PBS, 1% BSA, 0.3% Triton-X100, Hoechst (1:10,000)

incubate in 25 μL of primary antibody in blocking buffer at 4 deg C. over night

Primary Antibodies:
Plate A K48-Ub 1:20,000 (millipore 05-1307 Lot 2049282) Rabbit
   CHOP/Gadd153 1:2,000 (SC-7351) Mouse
Plate B P53 1:2,000 (SC-6243) Rabbit
   p62/SQSTM1 1:2,000 (SC-28359) Mouse
   overnight at 4 deg C.

Secondary Antibodies:
AlexaFluor488 Goat anti-Rabbit 1:2,000 (Life Tech A11008)
AlexaFluor555 Goat anti-Mouse 1:2,000 (Life Tech A21422)

Day 3/4

Black Plate Staining (Cont):

wash black plates 3× in 50 μL PBS (˜5 min each)
incubate in 25 μL of secondary antibody (1:2,000) in blocking buffer for 1-2 hrs at room temp (alexafluor488-anti-Rabbit/alexafluor555-anti-Mouse)

wash 4× in 50 μL PBS (˜5 min each)

leave plates in PBS for imaging

clean bottom of plates with 70% EtOH

Imaging:

Image plates in high content microscope with 405 nm, 488 nm and 555 nm filters

Data Analysis:
Nuclear counts and cellular intensities of each markers are measured using Hoechst as a nuclear marker with an automated image analysis protocol using Matlab software (Math Works)

Day 5

Viability Assay:

Thaw an aliquot of frozen cell titer glo (Promega G7572) at room temperature.

Add 45 mL of NaCl/PBS solution to 5 ml of cell titer glo (10×).

Remove white plates from incubator, leave at room temp for 30 minutes.

Add 25 μL of diluted cell titer glo to each well.

Shake plate for >1 minute.

Incubate plate for >5 minutes to stabilize luminescence.

Luminescence is stable for up to 3 hours.

Read luminescence on plate reader

### Results of Biological Assays

The results of the primary assayS conducted with selected compounds of the invention show that the tricyclic compounds of the invention display significant inhibitory activity (IC₅₀) against the enzymatic action of p97 toward its natural substrate. Some of these compounds also have greater potency in cell based assays and have in vitro pharmacokinetic properties consistent with good oral bioavailability.

Table I presents the results of several of these assays conducted upon the compounds of the invention.

TABLE 1

| | | 97 ADP | A549- | |
|---|---|---|---|---|
| | | Glo | CellTitre | A549-K48 |
| | | Assay: | Glo: | Cell |
| | ChemDraw | IC50 | IC50 | intensity: |
| Compound | IUPAC | (uM) | (uM) | IC50 (uM) |
| 001 | 1-(4- | 0.016 | 1.342 | 0.736 |
| | (benzylamino)- | | | |
| | 6,8,9,10,11,11a- | | | |
| | hexahydro-5H- | | | |
| | pyrimido[4,5- | | | |
| | a]quinolizin-2- | | | |
| | yl)-2-methyl-1H- | | | |
| | indole-4- | | | |
| | carboxamide | | | |
| 002 | 1-(4- | 0.015 | 2.693 | 6.624 |
| | (benzylamino)- | | | |
| | 5,6,8,9,10,10a- | | | |
| | hexahydropyrimido[5, | | | |
| | 4- | | | |
| | g]indolizin-2- | | | |
| | yl)-2-methyl-1H- | | | |
| | indole-4- | | | |
| | carboxamide | | | |
| 003 | 1-(4- | 0.025 | 2.342 | 0.642 |
| | (benzylamino)- | | | |
| | 7,8,9,10,10a,11- | | | |
| | hexahydro-5H- | | | |
| | pyrimido[4,5- | | | |
| | b]quinolizin-2- | | | |
| | yl)-2-methyl-1H- | | | |
| | indole-4- | | | |
| | carboxamide | | | |
| 004 | 1-(4- | 0.019 | 1.121 | 0.273 |
| | (benzylamino)- | | | |
| | 5,7,8,9,9a,10- | | | |
| | hexahydropyrimido[5, | | | |
| | 4- | | | |
| | f]indolizin-2-yl)- | | | |
| | 2-methyl-1H- | | | |
| | indole-4- | | | |
| | carboxamide | | | |

### Statements of the Invention

1. A tricyclic fused pyrimidine compound comprising Formula I

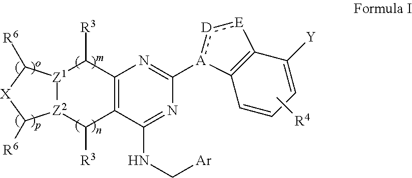

wherein:
m is an integer of 0, 1 or 2;
n is an integer of 0, 1 or 2;
the symbols m and n designate the ring to which they are attached as the m/n ring and sum of m and n is 1, 2 or 3 so as to provide a 5, 6 or 7 member m/n ring with the bridge with the o/p ring at the top, middle or bottom of the m/n ring;
o is an integer of 0, 1, 2 or 3;
p is an integer of 0, 1, 2 or 3;
the symbols o and p designate the ring to which they are attached as the o/p ring and the sum of o and p is 2, 3 or 4 so as to provide a 5, 6 or 7 member ring;
X is NR¹, O or C(R¹)₂;
Z¹ and Z² are each independently selected from N or CR² provided that when one of Z¹ and Z² is N, the other is CR²; and provided that when one of Z¹ and Z² is N and X is NR¹ or O, then the symbol p or o adjacent to the N of Z¹ or Z², is the integer 2 or 3;
each instance of R² and R⁴ is independently selected from hydrogen, a C₁ to C₄ straight or branched alkyl or halogen;
each instance of R³ and R⁶ is independently selected from hydrogen, a C₁ to C₄ straight or branched alkyl, halogen or a double bond O or S;
R¹ is independently selected from hydrogen, a C₁ to C₄ straight or branched alkyl, or an acyl group of C₁ to C₄ carbons in length;
Ar is phenyl, fluorophenyl or a monocyclic five or six member aromatic ring optionally containing one or two heteroatoms each independently selected from O, N, or S; or a single substituent version thereof wherein the substituent is selected from halogen or C₁ to C₄ straight or branched alkyl;
the group

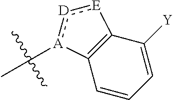

is designated as the P2 group wherein
A is N, C as a sp² carbon or CH as a sp³ carbon;
D is N or CR⁵ as a sp² carbon;
E is N, NR⁵ or CR⁵ as a sp² carbon;
the dotted lines between A-D and D-E indicate a single or a double bond according to the identities of A, D and E such that when A is N, the bond between A and D is single and the bond between D and E is double;
when A is C as a sp² carbon, the bond between A and D is double and the bond between D and E is single; when A is CH as a sp³ carbon, the bond between A and D is single and the bond between D and E is double;
each R⁵ is independently selected from hydrogen, C₁ to C₄ straight or branched alkyl, or C₁ to C₄ straight or branched alkoxy, provided that when D or E is NR⁵, R⁵ of NR⁵ is hydrogen or alkyl;
Y is —CO₂H, —CO₂R′, —CONH₂, —CONR′₂, —SO₃H, —SO₂NR′₂, —B(OH)₂, —B(OR′)₂, -tetrazolyl, —CH₂NR′₂, —CN, —CH₂OR′, —CH₂CO₂H, —CH₂CONR′₂ or —CH₂SO₂NR′₂, wherein each R′ is independently H or C₁ to C₄ straight or branched alkyl.
   2. A compound of statement 1 wherein the P2 group is selected from one of P2-A to P2-G:

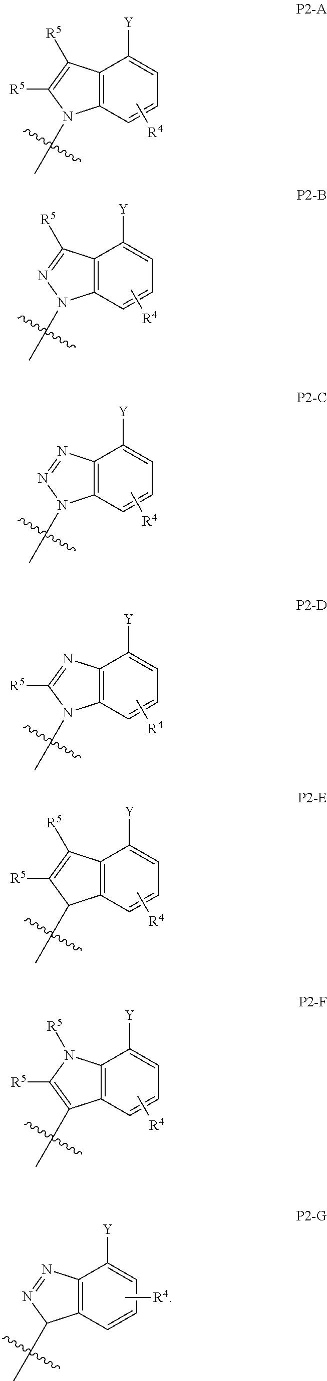

3. A compound of statement 2 wherein P2 is P2-A, P2-D or P2-G.
4. A compound of statement 2 wherein P2 is P2-A or P2-D.
5. A compound of statement 2 wherein P2 is P2-A.
6. A tricyclic fused pyrimidine compound of any one of statements 3, 4 or 5 comprising formula II

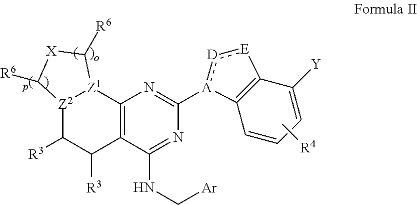

wherein: A, D, E, X, Y, Z¹, Z², R′, R¹, R², R³, R⁴, R⁵, R⁶, o and p are the same as recited in statement 1; m is 0 and n is 2.
   7. A tricyclic fused pyrimidine compound of any one of statements 3, 4 or 5 comprising formula III

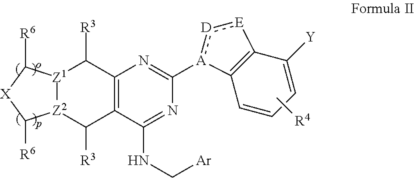

wherein: A, D, E, X, Y, Z¹, Z², R′, R¹, R², R³, R⁴, R⁵, R⁶, o and p are the same as recited in statement 1; m and n are both 1.
8. A tricyclic fused pyrimidine compound of any one of statements 3, 4 or 5 comprising formula IV

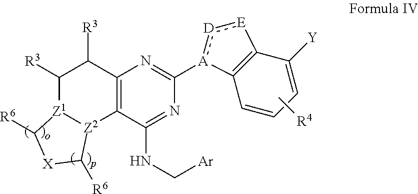

wherein: A, D, E, X, Y, Z¹, Z², R′, R¹, R², R³, R⁴, R⁵, R⁶, o and p are the same as recited in statement 1; m is 2 and n is 0.
   9. A tricyclic fused pyrimidine compound of any one of statements 3, 4 or 5 comprising formula V

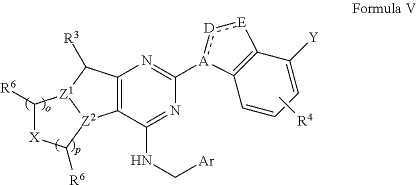

wherein A, D, E, X, Y, Z¹, Z², R′, R¹, R², R³, R⁴, R⁵, R⁶, o and p are the same as recited in statement 1; m is 1 and n is 0.
10. A tricyclic fused pyrimidine compound of any one of statements 3, 4 or 5 comprising formula VI

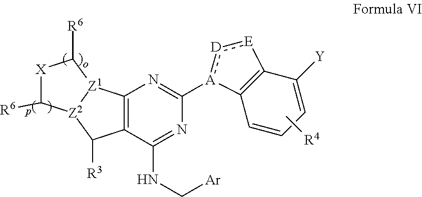

wherein A, D, E, X, Y, Z¹, Z², R′, R¹, R², R³, R⁴, R⁵, o and p are the same as recited in statement 1; m is 0 and n is 1.
11. A tricyclic fused pyrimidine compound of any one of statements 1-10 wherein each instance of R³ and R⁶ is independently selected from hydrogen, branched or straight alkyl or halogen.
12. A tricyclic fused pyrimidine compound of any one of statements 1-10 wherein each instance of R³ and R⁶ is independently selected from hydrogen or branched or straight alkyl.
13. A tricyclic fused pyrimidine compound of statement 6, wherein R³ and R⁶ are both hydrogen, Z¹ is N and Z² is CH, or Z¹ is CH and Z² is N, or Z¹ and Z² are both CH.
14. A tricyclic fused pyrimidine compound of statement 7, wherein R³ and R⁶ are both hydrogen, Z¹ is N and Z² is CH, or Z¹ is CH and Z² is N, or Z¹ and Z² are both CH.
15. A tricyclic fused pyrimidine compound of statement 8, wherein R³ and R⁶ are both hydrogen, Z¹ is N and Z² is CH, or Z¹ is CH and Z² is N, or Z¹ and Z² are both CH.
16. A tricyclic fused pyrimidine compound of statement 9, wherein R³ and R⁶ are both hydrogen, Z¹ is N and Z² is CH, or Z¹ is CH and Z² is N, or Z¹ and Z² are both CH.
17. A tricyclic fused pyrimidine compound of statement 10, wherein R³ and R⁶ are both hydrogen, Z¹ is N and Z² is CH, or Z¹ is CH and Z² is N, or Z¹ and Z² are both CH.
18. A tricyclic fused pyrimidine compound of any one of statements 1-17 wherein Z¹ and Z² are both CH.
19. A tricyclic fused pyrimidine compound of any one of statements 1-18 wherein X is CH₂, R³ and R⁶ are both hydrogen and the sum of o and p is 2.
20. A tricyclic fused pyrimidine compound of any one of statements 1-18 wherein X is CH₂, R³ and R⁶ are both hydrogen and the sum of o and p is 3.
21. A tricyclic fused pyrimidine compound of any one of statements 1-20 wherein R⁴ is hydrogen and each R⁵ of CR⁵ is independently selected from hydrogen, methyl or methoxy, and R⁵ of NR⁵ is hydrogen or methyl.
22. A tricyclic fused pyrimidine compound of any one of statements 1-21 wherein Y is carboxylic acid, carboxylic ester, carboxamido, sulfonoxy, sulfonamido, tetrazolyl, boronic acid or boronic ester, wherein the ester group of carboxylic ester or boronic ester is methyl or ethyl.
23. A tricyclic fused pyrimidine compound of statement 22 wherein D is C-Me, C—OMe, C-Et or C—OEt.
24. A tricyclic fused pyrimidine compound of any one of statements 18-23 wherein Formula II applies.
25. A tricyclic fused pyrimidine compound of any one of statements 18-23 wherein Formula III applies.
26. A tricyclic fused pyrimidine compound of any one of statements 18-23 wherein formula IV applies.
27. A tricyclic fused pyrimidine compound of any one of statements 1-18 and 21-26 wherein X is O.
28. A tricyclic fused pyrimidine compound of any one of statements 1-18 and 21-26 wherein X is NR¹.
29. A tricyclic fused pyrimidine compound of any one of statements 1-26 wherein X is CH₂.
30. A tricyclic fused pyrimidine compound of any one of statements 1-18 and 20-29 wherein the sum of o and p is 3 so that the op ring is a 6 member ring.
31. A tricyclic fused pyrimidine compound of any one of statements 1-17 and 19-29 wherein the sum of o and p is 2 so that the op ring is a 5 member ring.
32. A tricyclic fused pyrimidine compound of any one of statements 1-31 wherein each R³ is H and R⁴ is H.
33. A tricyclic fused pyrimidine compound of any one of statements 1-32 wherein Ar is phenyl, thiophenyl, pyridinyl, oxazole, furanyl or a mono-substituted version thereof wherein the substituent is selected from halogen or C₁ to C₄ straight or branched alkyl.
34. A tricyclic fused pyrimidine compound of statement 33 wherein the Ar is phenyl or fluorophenyl.
35. A tricyclic fused pyrimidine compound of statement 6 wherein o is 1; p is 2; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
36. A tricyclic fused pyrimidine compound of statement 7 wherein o is 1; p is 2; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
37. A tricyclic fused pyrimidine compound of statement 8 wherein o is 1; p is 2; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, or —B(OME)₂; Ar is phenyl or fluorophenyl.
38. A tricyclic fused pyrimidine compound of statement 6 wherein o is 2; p is 1; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
39. A tricyclic fused pyrimidine compound of statement 7 wherein o is 2; p is 1; Z¹ and Z² are both CR²; R², R³, R⁴ and R⁶ are all H; X is O or NH or NMe or CH₂; A is N; D is CH, CMe or COMe; E is CH Y is —COOH, —CONH₂, —SO₂NH₂, —CN, -tetrazolyl, —B(OH)₂ or —B(OME)₂; Ar is phenyl or fluorophenyl.
40. A tricyclic fused pyrimidine compound of statement 8 wherein o is 2; p is 1; Z¹ and Z² are both CR²; R², R³, R⁴ and R⁶ are all H; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
41. A tricyclic fused pyrimidine compound of statement 6 wherein o is 1; p is 2; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
42. A tricyclic fused pyrimidine compound of statement 7 wherein o is 1; p is 2; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
43. A tricyclic fused pyrimidine compound of statement 8 wherein o is 1; p is 2; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
44. A tricyclic fused pyrimidine compound of statement 6 wherein o is 2; p is 1; Z¹ is CR²; R², R³, R⁴ and R⁶ are all H; Z² is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
45. A tricyclic fused pyrimidine compound of statement 7 wherein o is 2; p is 1; Z¹ is CR²; R², R³, R⁴ and R⁶ are all H; Z¹ is N; X is O or NH or NMe or CH₂; A is N; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
46. A tricyclic fused pyrimidine compound of statement 8 wherein o is 2; p is 1; Z¹ is CR²; R², R³, R⁴ and R⁶ are all H; Z¹ is N; X is O or NH or NMe or CH₂; A is CH; D is CH, CMe or COMe; E is CH; Y is —COOH, —CONH₂, —SO₂NH₂, —CN, -tetrazolyl, —B(OH)₂ or —B(OME)₂; Ar is phenyl or fluorophenyl.
47. A tricyclic fused pyrimidine compound of statement 6 wherein o is 1; p is 2; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
48. A tricyclic fused pyrimidine compound of statement 7 wherein o is 1; p is 2; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH) or —B(OME)₂; Ar is phenyl or fluorophenyl.
49. A tricyclic fused pyrimidine compound of statement 8 wherein o is 1; p is 2; Z¹ and Z² are both CR²; R², R³, R⁴ and R⁶ are all H; X is O or NH or NMe or CH₂; A is CH; D is CMe or COMe; E is N; Y is —COOH, —CONH₂, —SO₂NH₂, —CN, -tetrazolyl, —B(OH)₂ or —B(OME)₂; Ar is phenyl or fluorophenyl.
50. A tricyclic fused pyrimidine compound of statement 6 wherein o is 2; p is 1; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
51. A tricyclic fused pyrimidine compound of statement 7 wherein o is 2; p is 1; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
52. A tricyclic fused pyrimidine compound of statement 8 wherein o is 2; p is 1; Z¹ and Z² are both CR²; R², R³, R⁴ and R⁶ are all H; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
53. A tricyclic fused pyrimidine compound of statement 6 wherein o is 1; p is 2; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
54. A tricyclic fused pyrimidine compound of statement 7 wherein o is 1; p is 2; Z¹ is CR²; R², R³, R⁴ and R⁶ are all H; Z¹ is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
55. A tricyclic fused pyrimidine compound of statement 8 wherein o is 1; p is 2; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
56. A tricyclic fused pyrimidine compound of statement 6 wherein o is 2; p is 1; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
57. A tricyclic fused pyrimidine compound of statement 7 wherein o is 2; p is 1; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
58. A tricyclic fused pyrimidine compound of statement 8 wherein o is 2; p is 1; Z¹ is CR²; R², R³, R⁴ and R⁶ are all H; Z² is N; X is O or NH or NMe or CH₂; A is CH; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
59. A tricyclic fused pyrimidine compound of statement 6 wherein o is 1; p is 2; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
60. A tricyclic fused pyrimidine compound of statement 7 wherein o is 1; p is 2; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; H, Ar is phenyl or fluorophenyl.
61. A tricyclic fused pyrimidine compound of statement 8 wherein o is 1; p is 2; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
62. A tricyclic fused pyrimidine compound of statement 6 wherein o is 2; p is 1; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
63. A tricyclic fused pyrimidine compound of statement 7 wherein o is 2; p is 1; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
64. A tricyclic fused pyrimidine compound of statement 8 wherein o is 2; p is 1; Z¹ and Z² are both CR²; R², R³, R⁴ and R⁶ are all H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
65. A tricyclic fused pyrimidine compound of statement 6 wherein o is 1; p is 2; Z¹ is CR²; R², R³, R⁴ and R⁶ are all H; Z² is N; R³ is H; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
66. A tricyclic fused pyrimidine compound of statement 7 wherein o is 1; p is 2; Z¹ is CR²; R², R³, R⁴ and R⁶ are all H; Z¹ is N; X is O or NH or NMe or CH₂; A is N; D is CH, CMe or COMe; E is N; Y is —COOH, —CONH₂. —SO₂NH₂, —CN, -tetrazolyl, —B(OH)₂ or —B(OME)₂; Ar is phenyl or fluorophenyl.
67. A tricyclic fused pyrimidine compound of statement 8 wherein o is 1; p is 2; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
68. A tricyclic fused pyrimidine compound of statement 6 wherein o is 2; p is 1; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
69. A tricyclic fused pyrimidine compound of statement 7 wherein o is 2; p is 1; Z¹ is CR²; R², R³, R⁴ and R⁶ are all H; Z² is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
70. A tricyclic fused pyrimidine compound of statement 8 wherein o is 2; p is 1; Z¹ is CR²; R², R³, R⁴ and R⁶ are all H; Z is N; X is O or NH or NMe or CH₂; A is N; D is CH, CMe or COMe; E is N; Y is —COOH, —CONH₂, —SO₂NH₂, —CN, -tetrazolyl, —B(OH)₂ or —B(OME)₂; Ar is phenyl or fluorophenyl.
71. A tricyclic fused pyrimidine compound of any one of statements 1-70 wherein Y is —CONH₂, —SO₂NH₂, -tetrazolyl or boronic acid or boronic ester.
72. A tricyclic fused pyrimidine compound of any one of statements 35-71 wherein D is CMe.
73. A tricyclic fused pyrimidine compound of any one of statements 35-71 wherein D is COMe.
74. A tricyclic fused pyrimidine compound of any one of statements 35-73 wherein X is O.
75. A tricyclic fused pyrimidine compound of any one of statements 35-73 wherein X is NH.
76. A tricyclic fused pyrimidine compound of any one of statements 35-73 wherein X is CH₂.
77. A tricyclic fused pyrimidine compound of any one of statements 1-34 wherein Z¹ and Z² are both CR² and R² is H.
78. A tricyclic fused pyrimidine compound of any one of statements 1-34 wherein one of Z¹ and Z² is N and R² is H.
79. A tricyclic fused pyrimidine compound of statement 77 or 78 wherein X is CH₂.
80. A tricyclic fused pyrimidine compound of statement 77 or 78 wherein X is O.
81. A compound according to statement 1 having the name:
1-(4-(benzylamino)-6,8,9,10,11,11a-hexahydro-5H-pyrimido[4,5-a]quinolizin-2-yl)-2-methyl-1H-indole-4-carboxamide
1-(4-((3-fluorobenzyl)amino)-6,8,9,10,11,11a-hexahydro-5H-pyrimido[4,5-a]quinolizin-2-yl)-2-methyl-1H-indole-4-carboxamide
(1-(4-(benzylamino)-6,8,9,10,11,11a-hexahydro-5H-pyrimido[4,5-a]quinolizin-2-yl)-2-methyl-1H-indol-4-yl)boronic acid
(1-(4-((3-fluorobenzyl)amino)-6,8,9,10,11,11a-hexahydro-5H-pyrimido[4,5-a]quinolizin-2-yl)-2-methyl-1H-indol-4-yl)boronic acid
1-(4-(benzylamino)-6,6a,7,8,9,10-hexahydro-5H-pyrimido[5,4-c]quinolizin-2-yl)-2-methyl-1H-indole-4-carboxamide
1-(4-((3-fluorobenzyl)amino)-6,6a,7,8,9,10-hexahydro-5H-pyrimido[5,4-c]quinolizin-2-yl)-2-methyl-1H-indole-4-carboxamide
(1-(4-(benzylamino)-6,6a,7,8,9,10-hexahydro-5H-pyrimido[5,4-c]quinolizin-2-yl)-2-methyl-1H-indol-4-yl)boronic acid
(1-(4-((3-fluorobenzyl)amino)-6,6a,7,8,9,10-hexahydro-5H-pyrimido[5,4-c]quinolizin-2-yl)-2-methyl-1H-indol-4-yl)boronic acid
1-(4-(benzylamino)-5a,6,7,8,9,11-hexahydro-5H-pyrimido[5,4-b]quinolizin-2-yl)-2-methyl-1H-indole-4-carboxamide
1-(4-((3-fluorobenzyl)amino)-5a,6,7,8,9,11-hexahydro-5H-pyrimido[5,4-b]quinolizin-2-yl)-2-methyl-1H-indole-4-carboxamide
(1-(4-(benzylamino)-5a,6,7,8,9,11-hexahydro-5H-pyrimido[5,4-b]quinolizin-2-yl)-2-methyl-1H-indol-4-yl)boronic acid
(1-(4-((3-fluorobenzyl)amino)-5a,6,7,8,9,11-hexahydro-5H-pyrimido[5,4-b]quinolizin-2-yl)-2-methyl-1H-indol-4-yl)boronic acid
1-(4-(benzylamino)-5,5a,6,8,9,11-hexahydropyrimido[5′,4′:4,5]pyrido[2,1-c][1,4]oxazin-2-yl)-2-methyl-1H-indole-4-carboxamide
1-(4-(benzylamino)-5a6,7,8,9,11-hexahydro-5H-pyrazino[1′,2′:1,6]pyrido[3,4-d]pyrimidin-2-yl)-2-methyl-1H-indole-4-carboxamide
1-(4-(benzylamino)-7,8,9,10,10a,11-hexahydro-5H-pyrimido[4,5-b]quinolizin-2-yl)-2-methyl-1H-indole-4-carboxamide
1-(4-((3-fluorobenzyl)amino)-7,8,9,10,10a,11-hexahydro-5H-pyrimido[4,5-b]quinolizin-2-yl)-2-methyl-1H-indole-4-carboxamide
(1-(4-(benzylamino)-7,8,9,10,10a,11-hexahydro-5H-pyrimido[4,5-b]quinolizin-2-yl)-2-methyl-1H-indol-4-yl)boronic acid
(1-(4-((3-fluorobenzyl)amino)-7,8,9,10,10a,11-hexahydro-5H-pyrimido[4,5-b]quinolizin-2-yl)-2-methyl-1H-indol-4-yl)boronic acid
1-(4-(benzylamino)-5,7,8,10,10a,11-hexahydropyrimido[4′,5′:4,5]pyrido[2,1-c][1,4]oxazin-2-yl)-2-methyl-1H-indole-4-carboxamide
1-(4-(benzylamino)-7,8,9,10,10a,11-hexahydro-5H-pyrazinol[1′,2′:1,6]pyrido[4,3-d]pyrimidin-2-yl)-2-methyl-1H-indole-4-carboxamide
1-(1-(benzylamino)-6,8,9,10,11,11a-hexahydro-5H-pyrimido[5,4-a]quinolizin-3-yl)-2-methyl-1H-indole-4-carboxamide
1-(1-((3-fluorobenzyl)amino)-6,8,9,10,11,11a-hexahydro-5H-pyrimido[5,4-a]quinolizin-3-yl)-2-methyl-1H-indole-4-carboxamide
1-(1-(benzylamino)-5,6,8,9,10,11,11a-hexahydropyrimido[5′,4′:3,4]pyrido[2,1-c][1,4]oxazin-3-yl)-2-methyl-1H-indole-4-carboxamide
1-(1-(benzylamino)-6,8,9,10,11,11a-hexahydro-5H-pyrazino[1′,2′:1,2]pyrido[4,3-d]pyrimidin-3-yl)-2-methyl-1H-indole-4-carboxamide
(1-(1-(benzylamino)-6,8,9,10,11,11a-hexahydro-5H-pyrimido[5,4-a]quinolizin-3-yl)-2-methyl-1H-indol-4-yl)boronic acid
(1-(1-((3-fluorobenzyl)amino)-6,8,9,10,11,11a-hexahydro-5H-pyrimido[5,4-a]quinolizin-3-yl)-2-methyl-1H-indol-4-yl)boronic acid
1-(1-(benzylamino)-6,6a,7,8,9,10-hexahydro-5H-pyrimido[4,5-c]quinolizin-3-yl)-2-methyl-1H-indole-4-carboxamide
1-(1-((3-fluorobenzyl)amino)-6,6a,7,8,9,10-hexahydro-5H-pyrimido[4,5-c]quinolizin-3-yl)-2-methyl-1H-indole-4-carboxamide
(1-(1-(benzylamino)-6,6a,7,8,9,10-hexahydro-5H-pyrimido[4,5-c]quinolizin-3-yl)-2-methyl-1H-indol-4-yl)boronic acid
(1-(1-((3-fluorobenzyl)amino)-6,6a,7,8,9,10-hexahydro-5H-pyrimido[4,5-c]quinolizin-3-yl)-2-methyl-1H-indol-4-yl)boronic acid
1-(1-((3-fluorobenzyl)amino)-5,6,8,9,10,10a-hexahydropyrimido[4,5-g]indolizin-3-yl)-2-methyl-1H-indole-4-carboxamide
1-(1-(benzylamino)-5,6,8,9,10,10a-hexahydropyrimido[4,5-g]indolizin-3-yl)-2-methyl-1H-indole-4-carboxamide
(1-(1-(benzylamino)-5,6,8,9,10,10a-hexahydropyrimido[4,5-g]indolizin-3-yl)-2-methyl-1H-indol-4-yl)boronic acid
(1-(1-((3-fluorobenzyl)amino)-5,6,8,9,10,10a-hexahydropyrimido[4,5-g]indolizin-3-yl)-2-methyl-1H-indol-4-yl)boronic acid
1-(4-(benzylamino)-5,7,8,9,9a,10-hexahydropyrimido[5,4-f]indolizin-2-yl)-2-methyl-1H-indole-4-carboxamide
1-(4-((3-fluorobenzyl)amino)-5,7,8,9,9a,10-hexahydropyrimido[5,4-f]indolizin-2-yl)-2-methyl-1H-indole-4-carboxamide
(1-(4-(benzylamino)-5,7,8,9,9a,10-hexahydropyrimido[5,4-f]indolizin-2-yl)-2-methyl-1H-indol-4-yl)boronic acid
(1-(4-((3-fluorobenzyl)amino)-5,7,8,9,9a,10-hexahydropyrimido[5,4-f]indolizin-2-yl)-2-methyl-1H-indol-4-yl)boronic acid
1-(4-(benzylamino)-5,5a,6,7,8,10-hexahydropyrimido[4,5-f]indolizin-2-yl)-2-methyl-1H-indole-4-carboxamide
1-(4-((3-fluorobenzyl)amino)-5,5a,6,7,8,10-hexahydropyrimido[4,5-f]indolizin-2-yl)-2-methyl-1H-indole-4-carboxamide
(1-(4-(benzylamino)-5,5a,6,7,8,10-hexahydropyrimido[4,5-f]indolizin-2-yl)-2-methyl-1H-indol-4-yl)boronic acid
(1-(4-((3-fluorobenzyl)amino)-5,5a,6,7,8,10-hexahydropyrimido[4,5-f]indolizin-2-yl)-2-methyl-1H-indol-4-yl)boronic acid
1-(4-(benzylamino)-5,6,8,9,10,10a-hexahydropyrimido[5,4-g]indolizin-2-yl)-2-methyl-1H-indole-4-carboxamide
1-(4-((3-fluorobenzyl)amino)-5,6,8,9,10,10a-hexahydropyrimido[5,4-g]indolizin-2-yl)-2-methyl-1H-indole-4-carboxamide
(1-(4-(benzylamino)-5,6,8,9,10,10a-hexahydropyrimido[5,4-g]indolizin-2-yl)-2-methyl-1H-indol-4-yl)boronic acid
(1-(4-((3-fluorobenzyl)amino)-5,6,8,9,10,10a-hexahydropyrimido[5,4-g]indolizin-2-yl)-2-methyl-1H-indol-4-yl)boronic acid
1-(4-(benzylamino)-5,6,6a,7,8,9-hexahydropyrimido[4,5-e]indolizin-2-yl)-2-methyl-1H-indole-4-carboxamide
1-(4-((3-fluorobenzyl)amino)-5,6,6a,7,8,9-hexahydropyrimido[4,5-e]indolizin-2-yl)-2-methyl-1H-indole-4-carboxamide
(1-(4-(benzylamino)-5,6,6a,7,8,9-hexahydropyrimido[4,5-e]indolizin-2-yl)-2-methyl-1H-indo-4-yl)boronic acid
(1-(4-((3-fluorobenzyl)amino)-5,6,6a7,8,9-hexahydropyrimido[4,5-e]indolizin-2-yl)-2-methyl-1H-indol-4-yl)boronic acid
1-(4-(benzylamino)-5,5a,6,7,8,9,9a,10-octahydrobenzo[g]quinazolin-2-yl)-2-methyl-1H-indole-4-carboxamide
(1-(4-(benzylamino)-5,5a,6,7,8,9,9a,10-octahydrobenzo[g]quinazolin-2-yl)-2-methyl-1H-indol-4-yl)boronic acid
1-(4-(benzylamino)-5a,6,8,9,9a,10-hexahydro-5H-pyrano[3,4-g]quinazolin-2-yl)-2-methyl-1H-indole-4-carboxamide
1-(4-(benzylamino)-5,5a,6,7,8,9,9a,10-octahydropyrido[3,4-g]quinazolin-2-yl)-2-methyl-1H-indole-4-carboxamide
(1-(4-(benzylamino)-5a,6,8,9,9a,10-hexahydro-5H-pyrano[3,4-g]quinazolin-2-yl)-2-methyl-1H-indol-1-yl)boronic acid
(1-(4-(benzylamino)-5,5a,6,7,8,9,9a,10-octahydropyrido[3,4-g]quinazolin-2-yl)-2-methyl-1H-indol-4-yl)boronic acid
1-(4-(benzylamino)-5a,6,7,9,9a,10-hexahydro-5H-pyrano[4,3-g]quinazolin-2-yl)-2-methyl-1H-indole-4-carboxamide
1-(4-(benzylamino)-5,5a,6,7,8,9,9a,10-octahydropyrido[4,3-g]quinazolin-2-yl)-2-methyl-1H-indole-4-carboxamide
(1-(4-(benzylamino)-5a,6,7,9,9a,10-hexahydro-5H-pyrano[4,3-g]quinazolin-2-yl)-2-methyl-1H-indol-4-yl)boronic acid
(1-(4-(benzylamino)-5,5a,6,7,8,9,9a,10-octahydropyrido[4,3-g]quinazolin-2-yl)-2-methyl-1H-indol-4-yl)boronic acid
1-(4-(benzylamino)-4b,5,6,7,8,10-hexahydropyrimido[5,4-a]indolizin-2-yl)-2-methyl-1H-indole-4-carboxamide
1-(4-(benzylamino)-6,7,8,9,9a,10-hexahydropyrimido[4,5-b]indolizin-2-yl)-2-methyl-1H-indole-4-carboxamide
(1-(4-(benzylamino)-6,7,8,9,9a,10-hexahydropyrimido[4,5-b]indolizin-2-yl)-2-methyl-1H-indo-4-yl)boronic acid
(1-(4-(benzylamino)-4b,5,6,7,8,10-hexahydropyrimido[5,4-a]indolizin-2-yl)-2-methyl-1H-indol-4-yl)boronic acid
1-(4-(benzylamino)-5,7,8,9,10,10a-hexahydropyrimido[4,5-a]indolizin-2-yl)-2-methyl-1H-indole-4-carboxamide
1-(4-(benzylamino)-5,5a,6,7,8,9-hexahydropyrimido[5,4-b]indolizin-2-yl)-2-methyl-1H-indole-4-carboxamide
(1-(4-(benzylamino)-5,5a,6,7,8,9-hexahydropyrimido[5,4-b]indolizin-2-yl)-2-methyl-1H-indol-4-yl)boronic acid
(1-(4-(benzylamino)-5,5a,6,7,8,9-hexahydropyrimido[5,4-b]indolizin-2-yl)-2-methyl-1H-indol-4-yl)boronic acid.
   82. A pharmaceutical composition comprising a pharmaceutically acceptable carrier and an amount of a compound of any one of statements 1 to 81 which is effective as an inhibitor of the AAA family member Valosin containing protein.
   83. A pharmaceutical composition according to statement 82 wherein the Valosin containing protein is in a human cell.
   84. A method of decreasing Valosin containing protein activity or decreasing degradation of a proteasome system substrate comprising administering to a patient an effective amount of a compound of any one of statements 1 to 81.
   85. A method of decreasing Valosin containing protein activity or degradation of a proteasome system substrate comprising administering to a patient an effective amount of a pharmaceutical composition of statements 82 or 83.
   86. A method according to statement 84 or 85 wherein the patient is a human.
   87. A method for treatment of a neoplastic malcondition associated with Valosin containing protein comprising administering to a patient in need thereof an effective amount of a compound according to one of statements 1 to 81 or an effective amount of a pharmaceutical composition according to statement 82 or 83.

### Summary Statements

The inventions, examples, biological assays and results described and claimed herein have may attributes and embodiments include, but hot limited to, those set forth or described or referenced in this application.

All patents, publications, scientific articles, web sites and other documents and material references or mentioned herein are indicative of the levels of skill of those skilled in the art to which the invention pertains, and each such referenced document and material is hereby incorporated by reference to the same extent as if it had been incorporated verbatim and set forth in its entirety herein. The right is reserved to physically incorporate into this specification any and all materials and information from any such patent, publication, scientific article, web site, electronically available information, text book or other referenced material or document.

The written description of this patent application includes all claims. All claims including all original claims are hereby incorporated by reference in their entirety into the written description portion of the specification and the right is reserved to physically incorporate into the written description or any other portion of the application any an all such claims. Thus, for example, under no circumstances may the patent be interpreted as allegedly not providing a written description for a claim on the assertion that the precise wording of the claim is not set forth in haec verba in written description portion of the patent. All features disclosed in this specification may be combined in any order and in any combination with any of the embodiments of Formula I.

While the invention has been described in conjunction with the detailed description thereof, the foregoing description is intended to illustrate and not limit the scope of the invention, which is defined by the scope of the appended claims. Thus, from the foregoing, it will be appreciated that, although specific nonlimiting embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred nonlimiting embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in nonlimiting embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by various nonlimiting embodiments and/or preferred nonlimiting embodiments and optional features, any and all modifications and variations of the concepts herein disclosed that may be resorted to by those skilled in the art are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, for example, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group. A Markush group is indicated by any one of a number of phrases including “selected from the group consisting of”, a series of atoms, groups or molecules ending with the penultimate term “or” and a series of atoms groups or molecules introduced by a phrase such as “selected from” or “chosen from.”

What is claimed is:

1. A tricyclic fused pyrimidine compound comprising Formula II or III 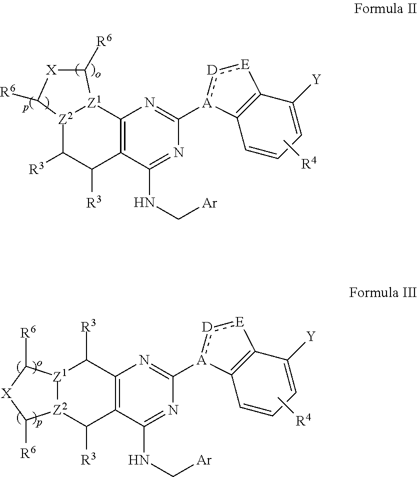
Wherein: the group 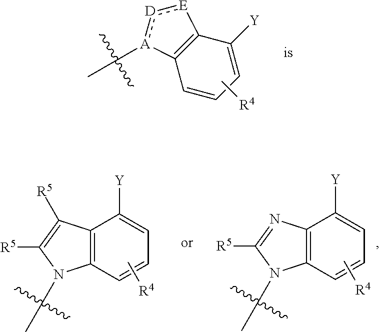
o and p are integers of 0, 1, 2 or 3;
the ring of Formula II or III with X is a five or six member ring such that the sum of o and p is 2 or 3;
One of Z¹ and Z² is CR² the other is N, or both are CR²;
X is NR¹ or O or C(R¹)₂ and when X is NR¹ or O, and one of Z¹ and Z² is N, the sum of o and p is 3 so that the ring with X is a six member ring, and the carbon designated by o or p of Formula II or III that is adjacent to Z¹ or Z² as N, the integer of the corresponding o or p is 2 or 3;
Each R¹ is independently selected from hydrogen, a C₁ to C₄ straight or branched alkyl, or an acyl group of C₁ to C₄ carbons in length;
Each R² is independently hydrogen or alkyl of 1 to 4 carbons;
R⁴ is hydrogen;
Y is —CO₂H, —CO₂R′, —CONH₂, —CONR′₂, —SO₃H, —SO₂NR′₂, —B(OH)₂, —B(OR′)₂, -tetrazolyl, —CH₂NR′₂, —CN, —CH₂OR′, —CH₂CO₂H, —CH₂CONR′₂ or —CH₂SO₂NR′₂, wherein each R′ is independently H or C₁ to C₄ straight or branched alkyl
Each instance of R⁵ is hydrogen or alkyl of 1 to 4 carbons;
Each instance of R³ and R⁶ is independently selected from hydrogen, a C₁ to C₄ straight or branched alkyl, halogen or a double bond O or S;
Ar is phenyl, thiophenyl, pyridinyl, oxazole, furanyl or a mono-substituted version thereof wherein the substituent is selected from halogen or C₁ to C₄ straight or branched alkyl.

2. A tricyclic fused pyrimidine compound of claim 1 wherein each instance of R³ and R⁶ is independently selected from hydrogen or branched or straight alkyl.

3. A tricyclic fused pyrimidine compound of claim 1 wherein R³ and R⁶ are both hydrogen.

4. A tricyclic fused pyrimidine compound of claim 1 wherein the sum of o and p is 3 such that the ring with X is a six member ring.

5. A tricyclic fused pyrimidine compound of claim 1 wherein Y is carboxylic acid, carboxylic ester, carboxamido, sulfonoxy, sulfonamido, tetrazolyl, boronic acid or boronic ester, wherein the ester group of carboxylic ester or boronic ester is methyl or ethyl.

6. A tricyclic fused pyrimidine compound of claim 1 wherein X is NR¹.

7. A tricyclic fused pyrimidine compound of claim 1 wherein X is C(R¹).

8. A tricyclic fused pyrimidine compound of claim 1 wherein R¹ is hydrogen.

9. A tricyclic fused pyrimidine compound of claim 1 wherein Ar is phenyl or fluorophenyl.

10. A compound of claim 1 having the name:
1-(4-(benzylamino)-6,8,9,10,11,11a-hexahydro-5H-pyrimido[4,5-a]quinolizin-2-yl)-2-methyl-1H-indole-4-carboxamide
1-(4-((3-fluorobenzyl)amino)-6,8,9,10,11,11a-hexahydro-5H-pyrimido[4,5-a]quinolizin-2-yl)-2-methyl-1H-indole-4-carboxamide
(1-(4-(benzylamino)-6,8,9,10,11,11a-hexahydro-5H-pyrimido[4,5-a]quinolizin-2-yl)-2-methyl-1H-indol-4-yl)boronic acid
(1-(4-((3-fluorobenzyl)amino)-6,8,9,10,11,11a-hexahydro-5H-pyrimido[4,5-a]quinolizin-2-yl)-2-methyl-1H-indol-4-yl)boronic acid
1-(4-(benzylamino)-6,6a,7,8,9,10-hexahydro-5H-pyrimido[5,4-c]quinolizin-2-yl)-2-methyl-1H-indole-4-carboxamide
1-(4-((3-fluorobenzyl)amino)-6,6a,7,8,9,10-hexahydro-5H-pyrimido[5,4-c]quinolizin-2-yl)-2-methyl-1H-indole-4-carboxamide
(1-(4-(benzylamino)-6,6a,7,8,9,10-hexahydro-5H-pyrimido[5,4-c]quinolizin-2-yl)-2-methyl-1H-indol-4-yl)boronic acid
(1-(4-((3-fluorobenzyl)amino)-6,6a,7,8,9,10-hexahydro-5H-pyrimido[5,4-c]quinolizin-2-yl)-2-methyl-1H-indol-4-yl)boronic acid
1-(4-(benzylamino)-5a,6,7,8,9,11-hexahydro-5H-pyrimido[5,4-b]quinolizin-2-yl)-2-methyl-1H-indole-4-carboxamide
1-(4-((3-fluorobenzyl)amino)-5a,6,7,8,9,11-hexahydro-5H-pyrimido[5,4-b]quinolizin-2-yl)-2-methyl-1H-indole-4-carboxamide
(1-(4-(benzylamino)-5a,6,7,8,9,11-hexahydro-5H-pyrimido[5,4-b]quinolizin-2-yl)-2-methyl-1H-indol-4-yl)boronic acid
(1-(4-((3-fluorobenzyl)amino)-5a,6,7,8,9,11-hexahydro-5H-pyrimido[5,4-b]quinolizin-2-yl)-2-methyl-1H-indol-4-yl)boronic acid
1-(4-(benzylamino)-7,8,9,10,10a,11-hexahydro-5H-pyrimido[4,5-b]quinolizin-2-yl)-2-methyl-1H-indole-4-carboxamide
1-(4-((3-fluorobenzyl)amino)-7,8,9,10,10a,11-hexahydro-5H-pyrimido[4,5-b]quinolizin-2-yl)-2-methyl-1H-indole-4-carboxamide
(1-(4-(benzylamino)-7,8,9,10,10a,11-hexahydro-5H-pyrimido[4,5-b]quinolizin-2-yl)-2-methyl-1H-indol-4-yl)boronic acid
(1-(4-((3-fluorobenzyl)amino)-7,8,9,10,10a,11-hexahydro-5H-pyrimido[4,5-b]quinolizin-2-yl)-2-methyl-1H-indol-4-yl)boronic acid
1-(1-(benzylamino)-6,8,9,10,11,11a-hexahydro-5H-pyrimido[5,4-a]quinolizin-3-yl)-2-methyl-1H-indole-4-carboxamide
1-(1-((3-fluorobenzyl)amino)-6,8,9,10,11,11a-hexahydro-5H-pyrimido[5,4-a]quinolizin-3-yl)-2-methyl-1H-indole-4-carboxamide
(1-(1-(benzylamino)-6,8,9,10,11,11a-hexahydro-5H-pyrimido[5,4-a]quinolizin-3-yl)-2-methyl-1H-indol-4-yl)boronic acid
(1-(1-((3-fluorobenzyl)amino)-6,8,9,10,11,11a-hexahydro-5H-pyrimido[5,4-a]quinolizin-3-yl)-2-methyl-1H-indol-4-yl)boronic acid
1-(1-(benzylamino)-6,6a,7,8,9,10-hexahydro-5H-pyrimido[4,5-c]quinolizin-3-yl)-2-methyl-1H-indole-4-carboxamide
1-(1-((3-fluorobenzyl)amino)-6,6a,7,8,9,10-hexahydro-5H-pyrimiido[4,5-c]quinolizin-3-yl)-2-methyl-1H-indole-4-carboxamide
(1-(1-(benzylamino)-6,6a,7,8,9,10-hexahydro-5H-pyrimido[4,5-c]quinolizin-3-yl)-2-methyl-1H-indol-4-yl)boronic acid
(1-(1-((3-fluorobenzyl)amino)-6,6a,7,8,9,10-hexahydro-5H-pyrimido[4,5-c]quinolizin-3-yl)-2-methyl-1H-indol-4-yl)boronic acid
1-(1-((3-fluorobenzyl)amino)-5,6,8,9,10,10a-hexahydropyrimido[4,5-g]indolizin-3-yl)-2-methyl-1H-indole-4-carboxamide
1-(1-(benzylamino)-5,6,8,9,10,10a-hexahydropyrimido[4,5-g]indolizin-3-yl)-2-methyl-1H-indole-4-carboxamide
(1-(1-(benzylamino)-5,6,8,9,10,10a-hexahydropyrimido[4,5-g]indolizin-3-yl)-2-methyl-1H-indol-4-yl)boronic acid
(1-(1-((3-fluorobenzyl)amino)-5,6,8,9,10,10a-hexahydropyrimido[4,5-g]indolizin-3-yl)-2-methyl-1H-indol-4-yl)boronic acid
1-(4-(benzylamino)-5,7,8,9,9a,10-hexahydropyrimido[5,4-f]indolizin-2-yl)-2-methyl-1H-indole-4-carboxamide
1-(4-((3-fluorobenzyl)amino)-5,7,8,9,9a,10-hexahydropyrimido[5,4-f]indolizin-2-yl)-2-methyl-1H-indole-4-carboxamide
(1-(4-(benzylamino)-5,7,8,9,9a,10-hexahydropyrimido[5,4-f]indolizin-2-yl)-2-methyl-1H-indol-4-yl)boronic acid
(1-(4-((3-fluorobenzyl)amino)-5,7,8,9,9a,10-hexahydropyrimido[5,4-f]indolizin-2-yl)-2-methyl-1H-indol-4-yl)boronic acid
1-(4-(benzylamino)-5,5a,6,7,8,10-hexahydropyrimido[4,5-f]indolizin-2-yl)-2-methyl-1H-indole-4-carboxamide
1-(4-((3-fluorobenzyl)amino)-5,5a,6,7,8,10-hexahydropyrimido[4,5-f]indolizin-2-yl)-2-methyl-1H-indole-4-carboxamide
(1-(4-(benzylamino)-5,5a,6,7,8,10-hexahydropyrimido[4,5-f]indolizin-2-yl)-2-methyl-1H-indol-4-yl)boronic acid
(1-(4-((3-fluorobenzyl)amino)-5,5a,6,7,8,10-hexahydropyrimido[4,5-f]indolizin-2-yl)-2-methyl-1H-indol-4-yl)boronic acid
1-(4-(benzylamino)-5,6,8,9,10,10a-hexahydropyrimido[5,4-g]indolizin-2-yl)-2-methyl-1H-indole-4-carboxamide
1-(4-((3-fluorobenzyl)amino)-5,6,8,9,10,10a-hexahydropyrimido[5,4-g]indolizin-2-yl)-2-methyl-1H-indole-4-carboxamide
(1-(4-(benzylamino)-5,6,8,9,10,10a-hexahydropyrimido[5,4-g]indolizin-2-yl)-2-methyl-1H-indol-4-yl)boronic acid
(1-(4-((3-fluorobenzyl)amino)-5,6,8,9,10,10a-hexahydropyrimido[5,4-g]indolizn-2-yl)-2-methyl-1H-indol-4-yl)boronic acid
1-(4-(benzylamino)-5,6,6a,7,8,9-hexahydropyrimido[4,5-e]indolizin-2-yl)-2-methyl-1H-indole-4-carboxamide
1-(4-((3-fluorobenzyl)amino)-5,6,6a,7,8,9-hexahydropyrimido[4,5-e]indolizin-2-yl)-2-methyl-1H-indole-4-carboxamide
(1-(4-(benzylamino)-5,6,6a,7,8,9-hexahydropyrimido[4,5-e]indolizin-2-yl)-2-methyl-1H-indol-4-yl)boronic acid
(1-(4-((3-fluorobenzyl)amino)-5,6,6a,7,8,9-hexahydropyrimido[4,5-e]indolizin-2-yl)-2-methyl-1H-indol-4-yl)boronic acid
1-(4-(benzylamino)-5,5a,6,7,8,9,9a,10-octahydrobenzo[g]quinazolin-2-yl)-2-methyl-1H-indole-4-carboxamide
(1-(4-(benzylamino)-5,5a,6,7,8,9,9a,10-octahydrobenzo[g]quinazolin-2-yl)-2-methyl-1H-indol-4-yl)boronic acid
1-(4-(benzylamino)-5a,6,8,9,9a,10-hexahydro-5H-pyrano[3,4-g]quinazolin-2-yl)-2-methyl-1H-indole-4-carboxamide
1-(4-(benzylamino)-5,5a,6,7,8,9,9a,10-octahydropyrido[3,4-g]quinazolin-2-yl)-2-methyl-1H-indole-4-carboxamide
(1-(4-(benzylamino)-5a,6,8,9,9a,10-hexahydro-5H-pyrano[3,4-g]quinazolin-2-yl)-2-methyl-1H-indol-4-yl)boronic acid
(1-(4-(benzylamino)-5,5a,6,7,8,9,9a,10-octahydropyrido[3,4-g]quinazolin-2-yl)-2-methyl-1H-indol-4-yl)boronic acid
1-(4-(benzylamino)-5a,6,7,9,9a,10-hexahydro-5H-pyrano[4,3-g]quinazolin-2-yl)-2-methyl-1H-indole-4-carboxamide
1-(4-(benzylamino)-5,5a,6,7,8,9,9a,10-octahydropyrido[4,3-g]quinazolin-2-yl)-2-methyl-1H-indole-4-carboxamide
(1-(4-(benzylamino)-5a,6,7,9,9a,10-hexahydro-5H-pyrano[4,3-g]quinazolin-2-yl)-2-methyl-1H-indol-4-yl)boronic acid
(1-(4-(benzylamino)-5,5a,6,7,8,9,9a,10-octahydropyrido[4,3-g]quinazolin-2-yl)-2-methyl-1H-indol-4-yl)boronic acid
1-(4-(benzylamino)-4b,5,6,7,8,10-hexahydropyrimido[5,4-a]indolizin-2-yl)-2-methyl-1H-indole-4-carboxamide
1-(4-(benzylamino)-6,7,8,9,9a,10-hexahydropyrimido[4,5-b]indolizin-2-yl)-2-methyl-1H-indole-4-carboxamide
(1-(4-(benzylamino)-6,7,8,9,9a,10-hexahydropyrimido[4,5-b]indolizin-2-yl)-2-methyl-1H-indol-4-yl)boronic acid
(1-(4-(benzylamino)-4b,5,6,7,8,10-hexahydropyrimido[5,4-a]indolizin-2-yl)-2-methyl-1H-indol-4-yl)boronic acid
1-(4-(benzylamino)-5,7,8,9,10,10a-hexahydropyrimido[4,5-a]indolizin-2-yl)-2-methyl-1H-indole-4-carboxamide
1-(4-(benzylamino)-5,5a,6,7,8,9-hexahydropyrimido[5,4-b]indolizin-2-yl)-2-methyl-1H-indole-4-carboxamide
(1-(4-(benzylamino)-5,5a,6,7,8,9-hexahydropyrimido[5,4-b]indolizin-2-yl)-2-methyl-1H-indol-4-yl)boronic acid
(1-(4-(benzylamino)-5,5a,6,7,8,9-hexahydropyrimido[5,4-b]indolizin-2-yl)-2-methyl-1H-indol-4-yl)boronic acid.

11. A pharmaceutical composition comprising a pharmaceutically acceptable carrier and an amount of a compound of claim 1 which is effective as an inhibitor of the AAA family member Valosin containing protein.

12. A pharmaceutical composition of claim 11 wherein the Valosin containing protein is in a human cell.

13. A method of decreasing Valosin containing protein activity or decreasing degradation of a proteasome system substrate comprising administering to a patient an effective amount of a compound of claim 1.

14. A method of decreasing Valosin containing protein activity or degradation of a proteasome system substrate comprising administering to a patient an effective amount of a pharmaceutical composition of claim 11.

15. A method of claim 13 wherein the patient is a human.

16. A method for treatment of a neoplastic malcondition associated with Valosin containing protein comprising administering to a patient in need thereof an effective amount of a compound of claim 1.

17. A method for treatment of a neoplastic malcondition associated with Valosin containing protein comprising administering to a patient in need thereof an effective amount of a pharmaceutical composition of claim 11.
